(19) United States
(12) Patent Application Publication
(10) Pub. No.: US 2025/0194337 A1
(43) Pub. Date: Jun. 12, 2025

(54) ORGANIC COMPOUND AND ORGANIC LIGHT-EMITTING DEVICE COMPRISING SAME

(71) Applicant: SFC CO., LTD., Cheongju-si (KR)

(72) Inventors: Si-In KIM, Cheongju-si (KR); Se-jin LEE, Cheongju-si (KR); Seok-bae PARK, Cheongju-si (KR); Hee-dae KIM, Cheongju-si (KR); Yeong-tae CHOI, Cheongju-si (KR); Ji-yung KIM, Cheongju-si (KR); Kyung-tae KIM, Cheongju-si (KR); Kyeong-hyeon KIM, Cheongju-si (KR); Tae-gyun LEE, Cheongju-si (KR); Joon-ho KIM, Cheongju-si (KR)

(73) Assignee: SFC CO., LTD., Cheongju-si (KR)

(21) Appl. No.: 18/845,684

(22) PCT Filed: Mar. 8, 2023
(86) PCT No.: PCT/KR2023/003187

§ 371 Date: Sep. 10, 2024

(30) Foreign Application Priority Data
Mar. 11, 2022 (KR) 10-2022-0030523

(51) Int. Cl. H10K 50/12; C07D 498/04; H10K 85/60

(52) U.S. Cl. CPC H10K 50/12; C07D 498/04; H10K 85/622; H10K 85/6572; H10K 85/6574

(57) ABSTRACT

The present invention relates to a novel compound with a characteristic structure and a high-efficiency, long-lifetime organic light-emitting device including same as a host compound in the light-emitting layer. Employing a polycyclic compound having a characteristic structure with a pyrene derivative incorporated thereinto as a host in the light-emitting layer, the organic light-emitting device according to the present invention can be configured as a high-efficiency, long-lifetime organic light-emitting device with excellent luminous characteristics in emission efficiency and the like and thus can find industrially advantageous applications in illumination devices as well as various display devices such as flat panel, flexible, and wearable displays.

### TECHNICAL FIELD

The present invention relates to a novel compound having a characteristic structure and a high-efficiency long-life organic light-emitting device comprising the same as a light-emitting layer host compound.

### BACKGROUND ART

Organic light emitting devices are self-luminous devices in which electrons injected from an electron injecting electrode (cathode) recombine with holes injected from a hole injecting electrode (anode) in a light emitting layer to form excitons, which emit light while releasing energy. Such organic light emitting devices have the advantages of low driving voltage, high luminance, large viewing angle, and short response time and can be applied to full-color light emitting flat panel displays. Due to these advantages, organic light emitting devices have received attention as next-generation light sources.

The above characteristics of organic light emitting devices are achieved by structural optimization of organic layers of the devices and are supported by stable and efficient materials for the organic layers, such as hole injecting materials, hole transport materials, light emitting materials, electron transport materials, electron injecting materials, and electron blocking materials. However, more research still needs to be done to develop structurally optimized structures of organic layers for organic light emitting devices and stable and efficient materials for organic layers of organic light emitting devices.

Particularly, for maximum efficiency in a light emitting layer, an appropriate combination of energy band gaps of a host and a dopant is required such that holes and electrons migrate to the dopant through stable electrochemical paths to form excitons.

### DETAILED DESCRIPTION OF THE INVENTION

### Problems to be Solved by the Invention

Therefore, the present invention is intended to provide a host material having a characteristic structure employed in a light emitting layer in an organic light emitting device, and a high-efficiency and long-lifetime organic light emitting device with significantly improved emission efficiency characteristics by employing the same.

### Means for Solving the Problems

One aspect of the present invention provides a compound represented by the following [Formula 1] or [Formula 2] employed as a host compound of an organic layer, preferably a light emitting layer, in a device.

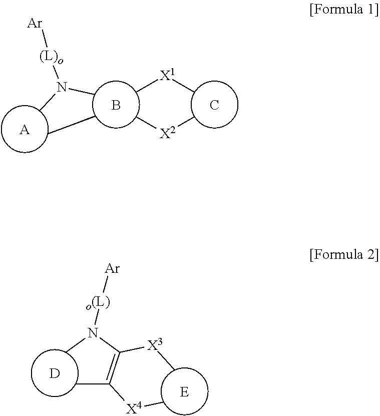

In [Formula 1] or [Formula 2], Ar is represented by the following [Structural Formula A].

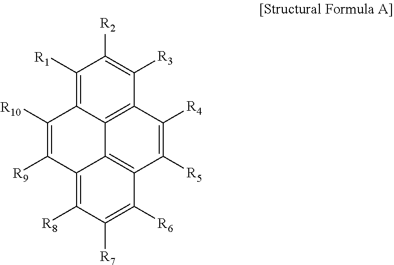

Specific structures of [Formula 1], [Formula 2] and [Structural Formula A], and a definition of each substituent will be described later.

Another aspect of the present invention provides an organic light emitting device including a first electrode, a second electrode opposite to the first electrode, and a light emitting layer interposed between the first electrode and the second electrode, wherein the light emitting layer includes the compound of [Formula 1] or [Formula 2] as a host.

According to one embodiment of the present invention, there is provided a high-efficiency and long-lifetime organic light emitting device with significantly improved emission efficiency characteristics by employing a compound selected from the following [Formula D-1] to [Formula D-10] as a dopant while including the compound of [Formula 1] or [Formula 2] as a host in a light emitting layer.

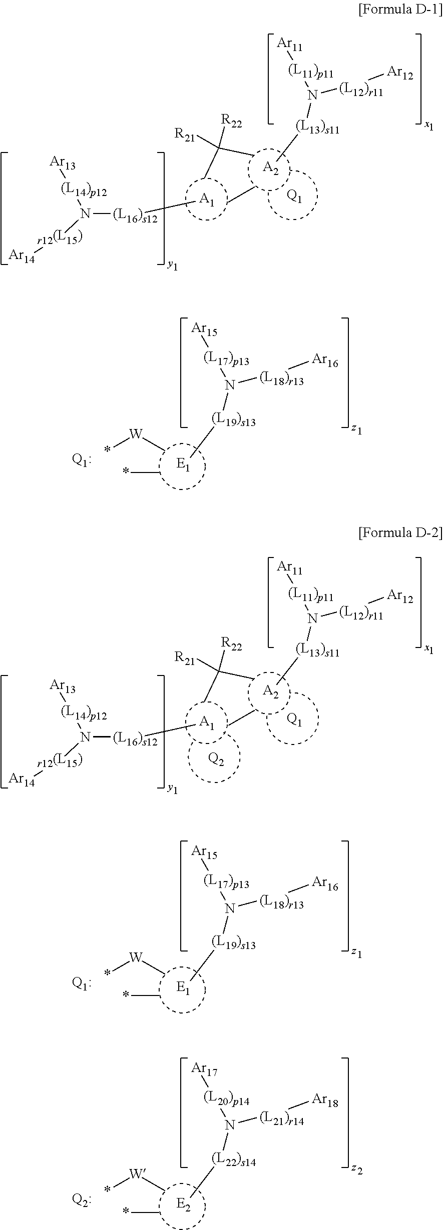

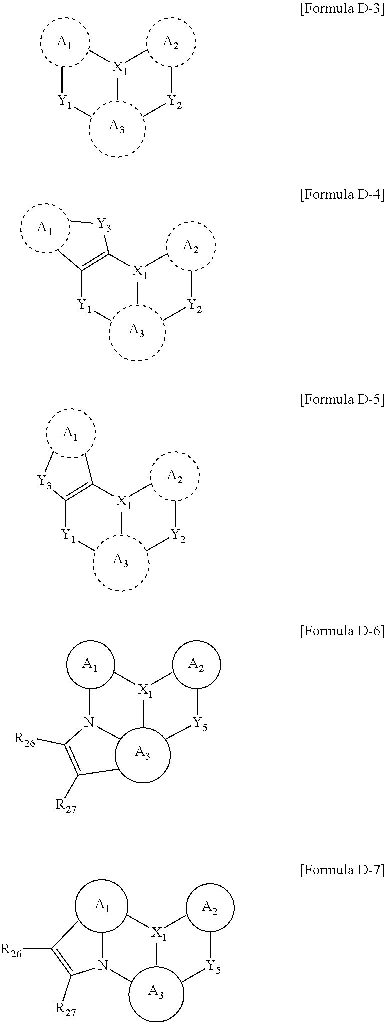

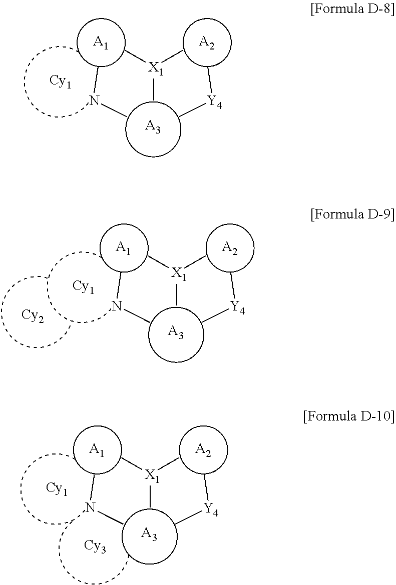

Specific structures of [Formula D-1] to [Formula D-10], and a definition of each substituent will be described later.

### Effects of the Invention

An organic light emitting device according to the present invention can be embodied as a high-efficiency and long-lifetime organic light emitting device with excellent luminous characteristics in emission efficiency and the like by employing a polycyclic compound having a characteristic structure with a pyrene derivative incorporated thereinto as a host in a light emitting layer, and therefore, is useful in illumination devices as well as various display devices such as flan panel, flexible and wearable displays.

### BEST MODE FOR CARRYING OUT THE INVENTION

The present invention will now be described in more detail.

One aspect of the present invention is directed to a compound represented by Formula 1 or Formula 2:

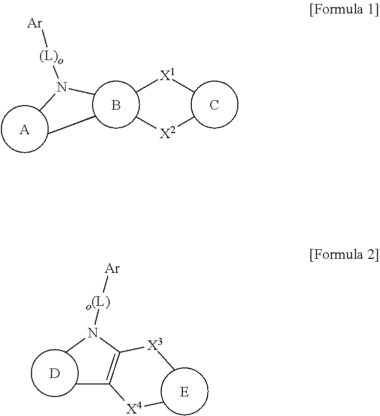

in [Formula 1] and [Formula 2],
rings A to E are each independently substituted or unsubstituted C₆-C₂₀ aryl.
X¹ to X⁴ are each independently a single bond, or CRR′, O, S or NR, however, cases where both X¹ and X² in [Formula 1] are a single bond and both X³ and X⁴ in [Formula 2] are a single bond are excluded.
R and R′ are each independently selected from hydrogen, deuterium, halogen, cyano, substituted or unsubstituted C₁-C₃₀ alkyl, substituted or unsubstituted C₃-C₃₀ cycloalkyl, substituted or unsubstituted C₁-C₃₀ heterocycloalkyl, substituted or unsubstituted C₆-C₅₀ aryl, substituted or unsubstituted C₂-C₅₀ heteroaryl, substituted or unsubstituted C₀-C₃₀ amine, substituted or unsubstituted C₀-C₃₀ silyl, substituted or unsubstituted C₁-C₃₀ alkoxy and substituted or unsubstituted C₆-C₅₀ aryloxy.
R and R′ in CRR′ are optionally linked to each other to form an alicyclic, aromatic monocyclic or polycyclic ring.
Ls are each independently a single bond, or substituted or unsubstituted C₁-C₁₀ alkylene, substituted or unsubstituted C₆-C₂₀ arylene, or substituted or unsubstituted C₅-C₂₀ heteroarylene, o is from 1 to 3, and when o is 2 or greater, a plurality of Ls may each be the same as or different from each other.

According to one embodiment of the present invention, L may be substituted or unsubstituted C₆-C₂₀ arylene, and preferably any one selected from the following [Structural Formula 1] to [Structural Formula 5].

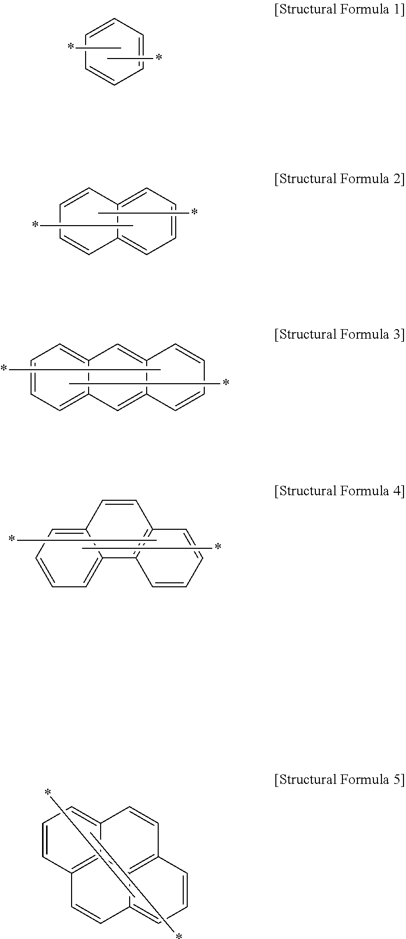

Hydrogen atoms at the carbon sites of the aromatic rings represented by [Structural Formula 1] to [Structural Formula 5] may be substituted with deuterium atoms.

Ar is represented by the following [Structural Formula A].

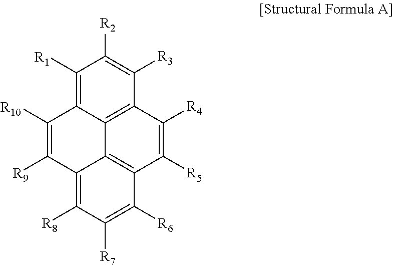

In [Structural Formula A],
any one of R₁ to R₁₀ is linked to L of [Formula 1] or [Formula 2].

The rest of R₁ to R₁₀ not linked to L are the same as or different from each other, and each independently selected from hydrogen, deuterium, halogen, cyano, substituted or unsubstituted C₁-C₃₀ alkyl, substituted or unsubstituted C₃-C₃₀ cycloalkyl, substituted or unsubstituted C₁-C₃₀ heterocycloalkyl, substituted or unsubstituted C₆-C₅₀ aryl, substituted or unsubstituted C₂-C₅₀ heteroaryl, substituted or unsubstituted C₀-C₃₀ amine, substituted or unsubstituted C₀-C₃₀ silyl, substituted or unsubstituted C₁-C₃₀ alkoxy and substituted or unsubstituted C₆-C₅₀ aryloxy.

According to one embodiment of the present invention, any one selected from R₁, R₃, R₆ and R₈ in [Structural Formula A] is optionally linked to the linker L of [Formula 1] or [Formula 2].

In addition, according to one embodiment of the present invention, the compound represented by [Formula 1] may be any one selected from the following [Formula 1-1] to [Formula 1-6].

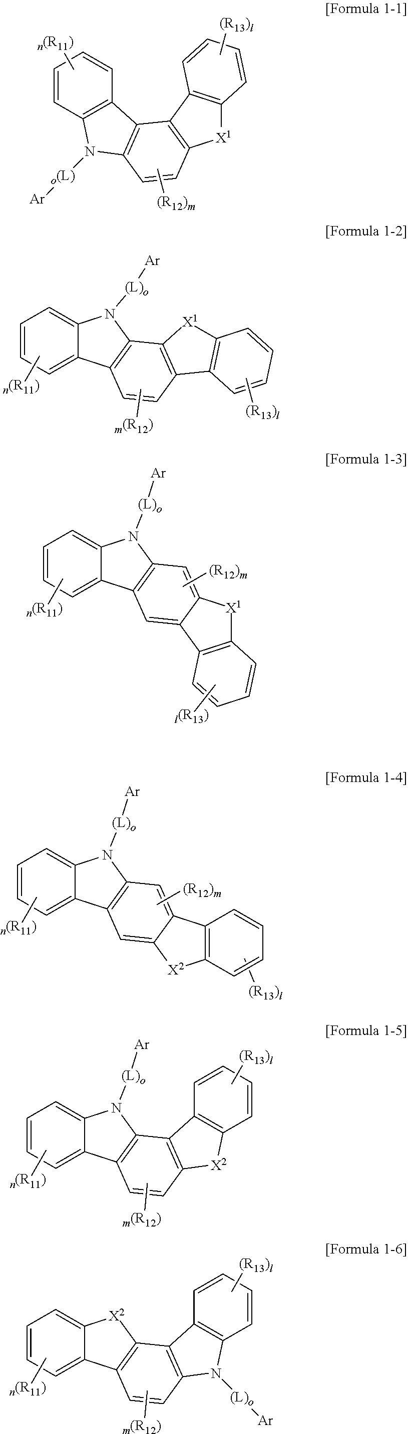

### In [Formula 1-1] to [Formula 1-6],

X₁ and X₂, L, o and Ar each have the same definitions as in [Formula 1].
R₁₁ to R₁₃ are the same as or different from each other, and each independently selected from hydrogen, deuterium, halogen, cyano, substituted or unsubstituted C₁-C₃₀ alkyl, substituted or unsubstituted C₃-C₃₀ cycloalkyl, substituted or unsubstituted C₁-C₃₀ heterocycloalkyl, substituted or unsubstituted C₆-C₅₀ aryl, substituted or unsubstituted C₂-C₅₀ heteroaryl, substituted or unsubstituted C₀-C₃₀ amine, substituted or unsubstituted C₀-C₃₀ silyl, substituted or unsubstituted C₁-C₃₀ alkoxy and substituted or unsubstituted C₆-C₅₀ aryloxy.
l and n are each an integer of 1 to 4, m is an integer of 1 to 2, and when 1, n and m are each 2 or greater, a plurality of R₁₁s to R₁₃s are each the same as or different from each other.

According to one embodiment of the present invention, the compound represented by [Formula 2] may be any one selected from the following [Formula 2-1] and [Formula 2-2].

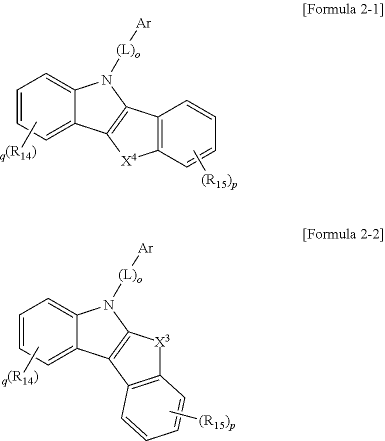

In [Formula 2-1] and [Formula 2-2],
X₃ and X₄, L, o and Ar have the same definitions as in [Formula 2].
R₁₄ and R₁₅ are the same as or different from each other, and each independently selected from hydrogen, deuterium, halogen, cyano, substituted or unsubstituted C₁-C₃₀ alkyl, substituted or unsubstituted C₃-C₃₀ cycloalkyl, substituted or unsubstituted C₁-C₃₀ heterocycloalkyl, substituted or unsubstituted C₆-C₅₀ aryl, substituted or unsubstituted C₂-C₅₀ heteroaryl, substituted or unsubstituted C₀-C₃₀ amine, substituted or unsubstituted C₀-C₃₀ silyl, substituted or unsubstituted C₁-C₃₀ alkoxy and substituted or unsubstituted C₆-C₅₀ aryloxy.
p and q are each an integer of 1 to 4, and when p and q are each 2 or greater, a plurality of R₁₄s and R₁₅s are each the same as or different from each other.

As used herein, the term “substituted” in the definition of the substituents in Formula 1, Formula 1-1 to Formula 1-6, Formula 2, Formula 2-1, Formula 2-2, and Structural Formula indicates substitution with one or more substituents selected from the group consisting of deuterium, cyano, halogen, hydroxyl, nitro, C₁-C₂₄ alkyl, C₃-C₂₄ cycloalkyl, C₁-C₂₄ haloalkyl, C₂-C₂₄ alkenyl, C₂-C₂₄ alkynyl, C₃-C₂₄ cycloalkyl, C₁-C₂₄ heteroalkyl, C₆-C₂₄ aryl, C₇-C₂₄ arylalkyl, C₇-C₂₄ alkylaryl, C₂-C₂₄ heteroaryl, C₂-C₂₄ heteroarylalkyl, C₁-C₂₄ alkoxy, C₁-C₂₄ alkylamino, C₁₂-C₂₄ diarylamino, C₂-C₂₄ diheteroarylamino, C₇-C₂₄ aryl(heteroaryl)amino, C₁-C₂₄ alkylsilyl, C₆-C₂₄ arylsilyl, C₆-C₂₄ aryloxy, and C₆-C₂₄ arylthionyl, more preferably deuterium, cyano, halogen, hydroxyl, nitro, C₁-C₁₂ alkyl, C₁-C₁₂ haloalkyl, C₂-C₁₂ alkenyl, C₂-C₁₂ alkynyl, C₃-C₁₂ cycloalkyl, C₁-C₁₂ heteroalkyl, C₆-C₁₈ aryl, C₇-C₂₀ arylalkyl, C₇-C₂₀ alkylaryl, C₂-C₁₈ heteroaryl, C₂-C₁₈ heteroarylalkyl, C₁-C₁₂ alkoxy, C₁-C₁₂ alkylamino, C₁₂-C₁₈ diarylamino, C₂-C₁₈ diheteroarylamino, C₇-C₁₈ aryl(heteroaryl)amino, C₁-C₁₂ alkylsilyl, C₆-C₁₈ arylsilyl, C₆-C₁₈ aryloxy, and C₆-C₁₈ arylthionyl, or a combination thereof. The term “unsubstituted” in the same definition indicates having no substituent. One or more hydrogen atoms in each of the substituents are optionally replaced by deuterium atoms.

A further aspect of the present invention is directed to an organic light emitting device including a first electrode, a second electrode opposite to the first electrode, and one or more organic layers interposed between the first and second electrodes wherein one of the organic layers, preferably a light emitting layer includes the pyrene derivative represented by Formula 1 or Formula 2.

According to one embodiment of the present invention, the organic light emitting device according to the present invention may also include a compound selected from the following [Formula D-1] to [Formula D-10] as a dopant, while including the compound represented by [Formula 1] or [Formula 2] as a host in the light emitting layer.

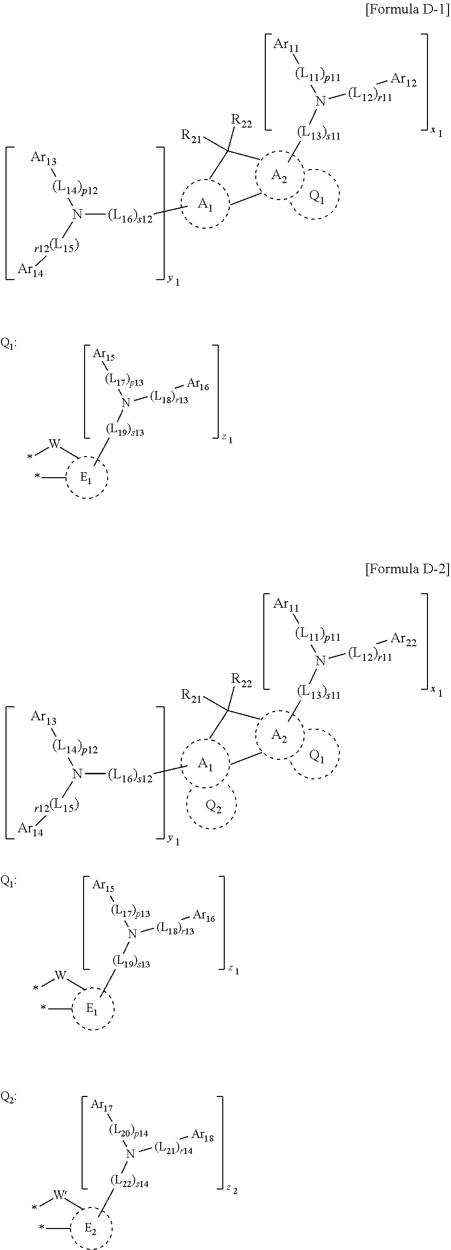

wherein A₁, A₂, E₁, and E₂ are each independently a substituted or unsubstituted C₆-C₅₀ aromatic hydrocarbon ring or a substituted or unsubstituted C₂-C₄₀ aromatic heterocyclic ring, with the proviso that two adjacent carbon atoms in the aromatic ring A₁ and two adjacent carbon atoms in the aromatic ring A₂ optionally form a 5-membered ring with the carbon atom to which R₂₁ and R₂₂ are attached, to form a fused ring, L₁₁ to L₂₂ are the same as or different from each other and are each independently selected from a single bond, substituted or unsubstituted C₁-C₆₀ alkylene, substituted or unsubstituted C₂-C₆₀ alkenylene, substituted or unsubstituted C₂-C₆₀ alkynylene, substituted or unsubstituted C₃-C₆₀ cycloalkylene, substituted or unsubstituted C₂-C₆₀ heterocycloalkylene, substituted or unsubstituted C₆-C₆₀ arylene, and substituted or unsubstituted C₂-C₆₀ heteroarylene, W, and W′ are each independently selected from N—R₂₃, CR₂₄R₂₅, SiR₂₆R₂₇, GeR₂₈R₂₉, O, S, and Se, R₂₁ to R₂₉ and Ar₁₁ to Ar₁₈ are the same as or different from each other and are each independently selected from hydrogen, deuterium, substituted or unsubstituted C₁-C₃₀ alkyl, substituted or unsubstituted C₆-C₅₀ aryl, substituted or unsubstituted C₂-C₃₀ alkenyl, substituted or unsubstituted C₂-C₂₀ alkynyl, substituted or unsubstituted C₃-C₃₀ cycloalkyl, substituted or unsubstituted C₅-C₃₀ cycloalkenyl, substituted or unsubstituted C₂-C₅₀ heteroaryl, substituted or unsubstituted C₂-C₃₀ heterocycloalkyl, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted C₁-C₃₀ alkylamine, substituted or unsubstituted C₅-C₃₀ arylamine, substituted or unsubstituted C₁-C₃₀ alkylsilyl, substituted or unsubstituted C₅-C₃₀ arylsilyl, substituted or unsubstituted C₁-C₃₀ alkylgermanium, substituted or unsubstituted C₁-C₃₀ arylgermanium, cyano, nitro, and halogen, with the proviso that R₅₁ and R₅₂ are optionally linked to each other to form an alicyclic or aromatic monocyclic or polycyclic ring in which carbon atoms are optionally substituted with one or more heteroatoms selected from the group consisting of N, O, P, Si, S, Ge, Se, and Te, p11 to p14, r11 to r14, and s11 to s14 are each independently an integer from 1 to 3, provided that when p11 is 2 or greater, the linkers L₁₁ are the same as or different from each other, when p12 is 2 or greater, the linkers L₁₄ are the same as or different from each other, when p13 is 2 or greater, the linkers L₁₇ are the same as or different from each other, p14 is 2 or greater, the linkers L₂₀ are the same as or different from each other, when r11 is 2 or greater, the linkers L₁₂ are the same as or different from each other, when r12 is 2 or greater, the linkers L₁₅ are the same as or different from each other, when r13 is 2 or greater, the linkers L₁₈ are the same as or different from each other, when r14 is 2 or greater, the linkers L₂₁ are the same as or different from each other, when s11 is 2 or greater, the linkers L₁₃ are the same as or different from each other, when s12 is 2 or greater, the linkers L₁₆ are the same as or different from each other, when s13 is 2 or greater, the linkers L₁₉ are the same as or different from each other, and when s14 is 2 or greater, the linkers L₂₂ are the same as or different from each other, x1 is 1, and y1 and z1 are each independently an integer of 0 or 1, with the proviso that Ar₁₁ and Ar₁₂ are optionally linked to each other to form a ring, Ar₁₃ and Ar₁₄ are optionally linked to each other to form a ring, Ar₁₅ and Ar₁₆ are optionally linked to each other to form a ring, and Ar₁₇ and Ar₁₈ are optionally linked to each other to form a ring, and with the proviso that two adjacent carbon atoms in the ring A₂ are optionally bonded to Q₁₁ at the asterisks (*) to form a fused ring, and with the proviso that two adjacent carbon atoms in the ring A₁ are optionally bonded to Q₁₂ at the asterisks (*) to form a fused ring and two adjacent carbon atoms in the ring A₂ are optionally bonded to Q₁₁ at the asterisks (*) to form a fused ring;

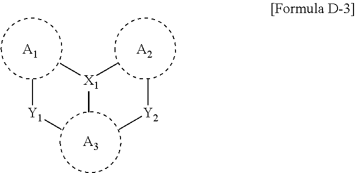

wherein X₁ is selected from B, P, and P═O, A₁ to A₃ are the same as or different from each other and are each independently a substituted or unsubstituted C₆-C₅₀ aromatic hydrocarbon ring or a substituted or unsubstituted C₂-C₄₀ aromatic heterocyclic ring, Y₁, and Y₂ are each independently selected from NR₂₁, CR₂₂R₂₃, O, S, Se, and SiR₂₄R₂₅, and R₂₁ to R₂₅ are the same as or different from each other and are each independently selected from hydrogen, deuterium, substituted or unsubstituted C₁-C₃₀ alkyl, substituted or unsubstituted C₆-C₅₀ aryl, substituted or unsubstituted C₃-C₃₀ cycloalkyl, substituted or unsubstituted C₂-C₅₀ heteroaryl, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted C₁-C₃₀ alkylamine, substituted or unsubstituted C₅-C₃₀ arylamine, substituted or unsubstituted C₁-C₃₀ alkylsilyl, substituted or unsubstituted C₅-C₃₀ arylsilyl, cyano, and halogen, with the proviso that each of R₂₁ to R₂₅ is optionally bonded to one or more rings selected from A₁ to A₃ to form an alicyclic or aromatic monocyclic or polycyclic ring;

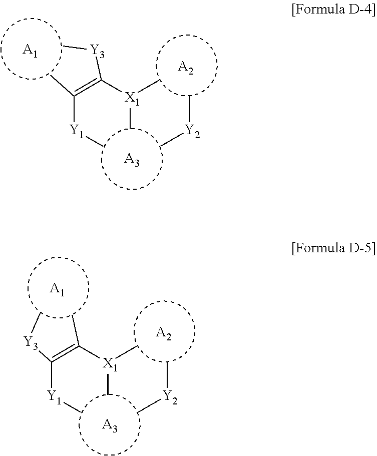

wherein Y₃ is selected from NR₂₁, CR₂₂R₂₃, O, S, Se, and SiR₂₄R₂₅, and X₁, Y₁, Y₂, A₁ to A₃, and R₂₁ to R₂₅ are as defined in Formula D3;

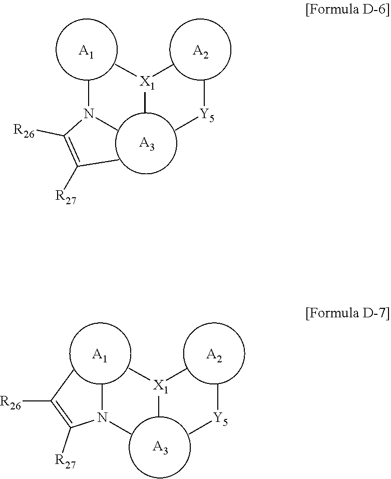

wherein Y₅ is selected from NR₂₁, CR₂₂R₂₃, O, S, Se, and SiR₂₄R₂₅, and X₁, A₁ to A₃, R₂₁ to R₂₅, R₂₆, and R₂₇ are as defined in Formula D3;

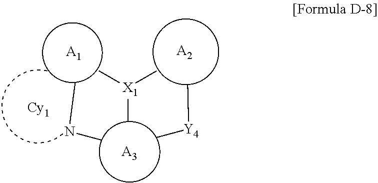

wherein Y₄ is selected from NR₂₁, CR₂₂R₂₃, O, S, Se, and SiR₂₄R₂₅, and X₁, A₁ to A₃, and R₂₁ to R₂₅ are as defined in Formula D3 and Cy₁ represents a ring formed by a substituted or unsubstituted C₂-C₅ alkylene group with the nitrogen (N) atom between the rings A₁, the aromatic carbon atom in the ring Q₁ to which the nitrogen (N) atom is bonded, and the aromatic carbon atom in the ring Q₁ to which Cy₁ is to be bonded;

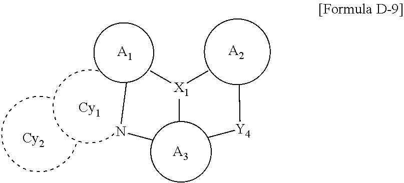

wherein Y₄ is selected from NR₂₁, CR₂₂R₂₃, O, S, Se, and SiR₂₄R₂₅, and X₁, A₁ to A₃, and R₂₁ to R₂₅ are as defined in Formula D3 and Cy₂ represents a saturated hydrocarbon ring attached to Cy₁ and formed by a substituted or unsubstituted C₂-C₅ alkylene group with carbon atoms in Cy₁; and

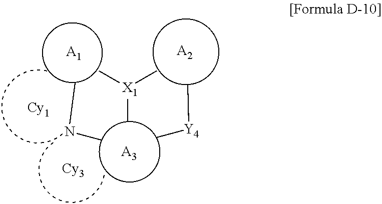

wherein Y₄ is selected from NR₂₁, CR₂₂R₂₃, O, S, Se, and SiR₂₄R₂₅, and X₁, A₁ to A₃, and R₂₁ to R₂₅ are as defined in Formula D3 and Cy₃ represents a ring formed by a substituted or unsubstituted C₁-C₄ alkylene group with the aromatic carbon atom in the ring A₃ to which Cy₃ is to be bonded, the nitrogen (N) atom between the rings A₃, the aromatic carbon atom in the ring A₃ to which the nitrogen (N) atom is bonded, and the carbon atom in Cy₁ to which the nitrogen (N) atom is bonded.

As used herein, the term “substituted” in the definition of the substituents in Formulas D1 to D10 indicates substitution with one or more substituents selected from the group consisting of deuterium, cyano, halogen, hydroxyl, nitro, C₁-C₂₄ alkyl, C₃-C₂₄ cycloalkyl, C₁-C₂₄ haloalkyl, C₂-C₂₄ alkenyl, C₂-C₂₄ alkynyl, C₃-C₂₄ cycloalkyl, C₁-C₂₄ heteroalkyl, C₆-C₂₄ aryl, C₇-C₂₄ arylalkyl, C₇-C₂₄ alkylaryl, C₂-C₂₄ heteroaryl, C₂-C₂₄ heteroarylalkyl, C₁-C₂₄ alkoxy, C₁-C₂₄ alkylamino, C₁₂-C₂₄ diarylamino, C₂-C₂₄ diheteroarylamino, C₇-C₂₄ aryl(heteroaryl)amino, C₁-C₂₄ alkylsilyl, C₆-C₂₄ arylsilyl, C₆-C₂₄ aryloxy, and C₆-C₂₄ arylthionyl, more preferably deuterium, cyano, halogen, hydroxyl, nitro, C₁-C₁₂ alkyl, C₁-C₁₂ haloalkyl, C₂-C₁₂ alkenyl, C₂-C₁₂ alkynyl, C₃-C₁₂ cycloalkyl, C₁-C₁₂ heteroalkyl, C₆-C₁₈ aryl, C₇-C₂₀ arylalkyl, C₇-C₂₀ alkylaryl, C₂-C₁₈ heteroaryl, C₂-C₁₈ heteroarylalkyl, C₁-C₁₂ alkoxy, C₁-C₁₂ alkylamino, C₁₂-C₁₈ diarylamino, C₂-C₁₈ diheteroarylamino, C₇-C₁₈ aryl(heteroaryl)amino, C₁-C₁₂ alkylsilyl, C₆-C₁₈ arylsilyl, C₆-C₁₈ aryloxy, and C₆-C₁₈ arylthionyl, or a combination thereof. The term “unsubstituted” in the same definition indicates having no substituent. One or more hydrogen atoms in each of the substituents are optionally replaced by deuterium atoms.

The content of the dopant in the light emitting layer is typically in the range of about 0.01 to about 20 parts by weight, based on about 100 parts by weight of the host but is not limited to this range.

The light emitting layer may further include one or more dopants other than the dopants represented by Formulas D1 to D10 and one or more hosts other than the host represented by Formula 1 to 2.

In the “substituted or unsubstituted C₁-C₃₀ alkyl”, “substituted or unsubstituted C₆-C₅₀ aryl”, etc., the number of carbon atoms in the alkyl or aryl group indicates the number of carbon atoms constituting the unsubstituted alkyl or aryl moiety without considering the number of carbon atoms in the substituent(s). For example, a phenyl group substituted with a butyl group at the para-position corresponds to a C₆ aryl group substituted with a C₄ butyl group.

As used herein, the term “bonded to an adjacent group to form a ring” means that the corresponding group combines with an adjacent group to form a substituted or unsubstituted alicyclic or aromatic ring and the term “adjacent substituent” may mean a substituent on an atom directly attached to an atom substituted with the corresponding substituent, a substituent disposed sterically closest to the corresponding substituent or another substituent on an atom substituted with the corresponding substituent. For example, two substituents substituted at the ortho position of a benzene ring or two substituents on the same carbon in an aliphatic ring may be considered “adjacent” to each other.

In the present invention, the alkyl groups may be straight or branched. Specific examples of the alkyl groups include, but are not limited to, methyl, ethyl, propyl, n-propyl, isopropyl, butyl, n-butyl, isobutyl, tert-butyl, sec-butyl, 1-methylbutyl, 1-ethylbutyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethylpropyl, 1,1-dimethylpropyl, isohexyl, 2-methylpentyl, 4-methylhexyl, and 5-methylhexyl groups.

The alkenyl group is intended to include straight and branched ones and may be optionally substituted with one or more other substituents. The alkenyl group may be specifically a vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, stilbenyl or styrenyl group but is not limited thereto.

The alkynyl group is intended to include straight and branched ones and may be optionally substituted with one or more other substituents. The alkynyl group may be, for example, ethynyl or 2-propynyl but is not limited thereto.

The cycloalkenyl group is a non-aromatic cyclic unsaturated hydrocarbon group having one or more carbon-carbon double bonds. The cycloalkenyl group may be, for example, cyclopropenyl, cyclobutenyl, cyclopentenyl, cyclohexenyl, cycloheptenyl, 1,3-cyclohexadienyl, 1,4-cyclohexadienyl, 2,4-cycloheptadienyl or 1,5-cyclooctadienyl but is not limited thereto.

The aromatic hydrocarbon rings or aryl groups may be monocyclic or polycyclic ones. Examples of the monocyclic aryl groups include, but are not limited to, phenyl, biphenyl, terphenyl, and stilbenyl groups. Examples of the polycyclic aryl groups include naphthyl, anthracenyl, phenanthrenyl, pyrenyl, perylenyl, tetracenyl, chrysenyl, fluorenyl, acenaphathcenyl, triphenylene, and fluoranthrene groups but the scope of the present invention is not limited thereto.

The aromatic heterocyclic rings or heteroaryl groups refer to aromatic groups containing one or more heteroatoms. Examples of the aromatic heterocyclic rings or heteroaryl groups include, but are not limited to, thiophene, furan, pyrrole, imidazole, thiazole, oxazole, oxadiazole, triazole, pyridyl, bipyridyl, pyrimidyl, triazine, triazole, acridyl, pyridazine, pyrazinyl, quinolinyl, quinazoline, quinoxalinyl, phthalazinyl, pyridopyrimidinyl, pyridopyrazinyl, pyrazinopyrazinyl, isoquinoline, indole, carbazole, benzoxazole, benzimidazole, benzothiazole, benzocarbazole, benzothiophene, dibenzothiophene, benzofuranyl, dibenzofuranyl, phenanthroline, thiazolyl, isoxazolyl, oxadiazolyl, thiadiazolyl, benzothiazolyl, and phenothiazinyl groups.

The aliphatic hydrocarbon rings refer to non-aromatic rings consisting only of carbon and hydrogen atoms. The aliphatic hydrocarbon ring is intended to include monocyclic and polycyclic ones and may be optionally substituted with one or more other substituents. As used herein, the term “polycyclic” means that the aliphatic hydrocarbon ring may be directly attached or fused to one or more other cyclic groups. The other cyclic groups may be aliphatic hydrocarbon rings and other examples thereof include aliphatic heterocyclic, aryl, and heteroaryl groups. Specific examples of the aliphatic hydrocarbon rings include, but are not limited to, cycloalkyl groups such as cyclopropyl, cyclobutyl, cyclopentyl, adamantyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, and cyclooctyl, cycloalkanes such as cyclohexane and cyclopentane, and cycloalkenes such as cyclohexene and cyclobutene.

The aliphatic heterocyclic rings refer to aliphatic rings containing one or more heteroatoms such as O, S, Se, N, and Si. The aliphatic heterocyclic ring is intended to include monocyclic or polycyclic ones and may be optionally substituted with one or more other substituents. As used herein, the term “polycyclic” means that the aliphatic heterocyclic ring such as heterocycloalkyl, heterocycloalkane or heterocycloalkene may be directly attached or fused to one or more other cyclic groups. The other cyclic groups may be aliphatic heterocyclic rings and other examples thereof include aliphatic hydrocarbon rings, aryl groups, and heteroaryl groups.

The mixed aliphatic-aromatic rings or the mixed aliphatic-aromatic cyclic groups refer to structures in which two or more rings are fused together and which are overall non-aromatic. The mixed aliphatic-aromatic polycyclic rings may contain one or more heteroatoms selected from N, O, P, and S other than carbon atoms (C).

The alkoxy group may be specifically a methoxy, ethoxy, propoxy, isobutyloxy, sec-butyloxy, pentyloxy, iso-amyloxy or hexyloxy group but is not limited thereto.

The silyl group is intended to include —SiH₃, alkylsilyl, arylsilyl, alkylarylsilyl, arylheteroarylsilyl, and heteroarylsilyl. The arylsilyl refers to a silyl group obtained by substituting one, two or three of the hydrogen atoms in —SiH₃ with aryl groups. The alkylsilyl refers to a silyl group obtained by substituting one, two or three of the hydrogen atoms in —SiH₃ with alkyl groups. The alkylarylsilyl refers to a silyl group obtained by substituting one of the hydrogen atoms in —SiH₃ with an alkyl group and the other two hydrogen atoms with aryl groups or substituting two of the hydrogen atoms in —SiH₃ with alkyl groups and the remaining hydrogen atom with an aryl group. The arylheteroarylsilyl refers to a silyl group obtained by substituting one of the hydrogen atoms in —SiH₃ with an aryl group and the other two hydrogen atoms with heteroaryl groups or substituting two of the hydrogen atoms in —SiH₃ with aryl groups and the remaining hydrogen atom with a heteroaryl group. The heteroarylsilyl refers to a silyl group obtained by substituting one, two or three of the hydrogen atoms in —SiH₃ with heteroaryl groups. The arylsilyl group may be, for example, substituted or unsubstituted monoarylsilyl, substituted or unsubstituted diarylsilyl, or substituted or unsubstituted triarylsilyl. The same applies to the alkylsilyl and heteroarylsilyl groups.

Each of the aryl groups in the arylsilyl, heteroarylsilyl, and arylheteroarylsilyl groups may be a monocyclic or polycyclic one. Each of the heteroaryl groups in the arylsilyl, heteroarylsilyl, and arylheteroarylsilyl groups may be a monocyclic or polycyclic one.

Specific examples of the silyl groups include trimethylsilyl, triethylsilyl, triphenylsilyl, trimethoxysilyl, dimethoxyphenylsilyl, diphenylmethylsilyl, diphenylvinylsilyl, methylcyclobutylsilyl, and dimethylfurylsilyl. One or more of the hydrogen atoms in each of the silyl groups may be substituted with the substituents mentioned in the aryl groups.

The amine group is intended to include —NH₂, alkylamine, arylamine, arylheteroarylamine, and heteroarylamine. The arylamine refers to an amine group obtained by substituting one or two of the hydrogen atoms in —NH₂ with aryl groups. The alkylamine refers to an amine group obtained by substituting one or two of the hydrogen atoms in —NH₂ with alkyl groups. The alkylarylamine refers to an amine group obtained by substituting one of the hydrogen atoms in —NH₂ with an alkyl group and the other hydrogen atom with an aryl group. The arylheteroarylamine refers to an amine group obtained by substituting one of the hydrogen atoms in —NH₂ with an aryl group and the other hydrogen atom with a heteroaryl group. The heteroarylamine refers to an amine group obtained by substituting one or two of the hydrogen atoms in —NH₂ with heteroaryl groups. The arylamine may be, for example, substituted or unsubstituted monoarylamine, substituted or unsubstituted diarylamine, or substituted or unsubstituted triarylamine. The same applies to the alkylamine and heteroarylamine groups.

Each of the aryl groups in the arylamine, heteroarylamine, and arylheteroarylamine groups may be a monocyclic or polycyclic one. Each of the heteroaryl groups in the arylamine, heteroarylamine, and arylheteroarylamine groups may be a monocyclic or polycyclic one.

The germanium group is intended to include —GeH₃, alkylgermanium, arylgermanium, heteroarylgermanium, alkylarylgermanium, alkylheteroarylgermanium, and arylheteroarylgermanium. The definitions of the substituents in the germanium groups follow those described for the silyl groups, except that the silicon (Si) atom in each silyl group is changed to a germanium (Ge) atom.

Specific examples of the germanium groups include trimethylgermane, triethylgermane, triphenylgermane, trimethoxygermane, dimethoxyphenylgermane, diphenylmethylgermane, diphenylvinylgermane, methylcyclobutylgermane, and dimethylfurylgermane. One or more of the hydrogen atoms in each of the germanium groups may be substituted with the substituents mentioned in the aryl groups.

The cycloalkyl, aryl, and heteroaryl groups in the cycloalkyloxy, aryloxy, heteroaryloxy, cycloalkylthioxy, arylthioxy, and heteroarylthioxy groups are the same as those exemplified above. Specific examples of the aryloxy groups include, but are not limited to, phenoxy, p-tolyloxy, m-tolyloxy, 3,5-dimethylphenoxy, 2,4,6-trimethylphenoxy, p-tert-butylphenoxy, 3-biphenyloxy, 4-biphenyloxy, 1-naphthyloxy, 2-naphthyloxy, 4-methyl-1-naphthyloxy, 5-methyl-2-naphthyloxy, 1-anthryloxy, 2-anthryloxy, 9-anthryloxy, 1-phenanthryloxy, 3-phenanthryloxy, and 9-phenanthryloxy groups. Specific examples of the arylthioxy groups include, but are not limited to, phenylthioxy, 2-methylphenylthioxy, and 4-tert-butylphenylthioxy groups.

The halogen group may be, for example, fluorine, chlorine, bromine or iodine.

According to one embodiment of the present invention, the organic compound represented by Formula 1 may be selected from the following compounds [1] to [114].

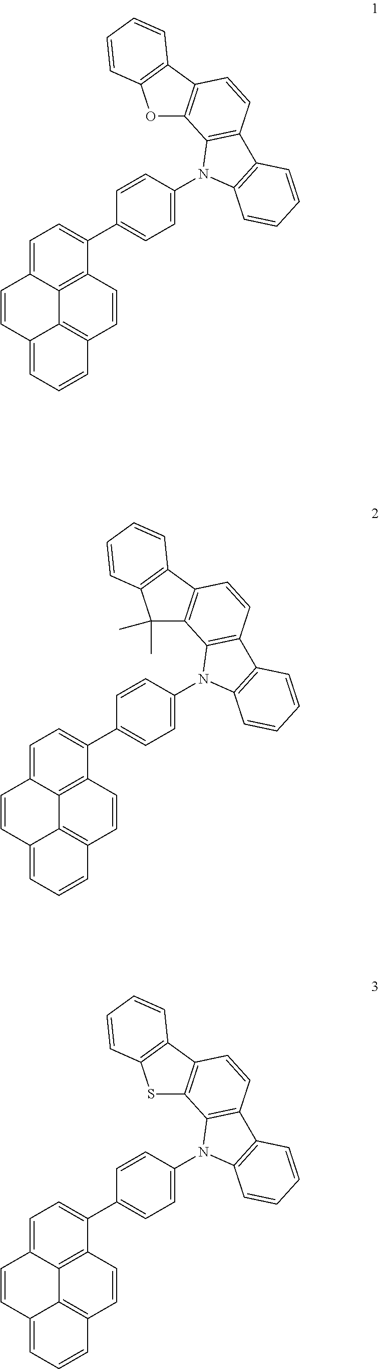 [SMILES: c1cc2ccc3ccc(-c4ccc(-n5c6ccccc6c6ccc7c8ccccc8oc7c65)cc4)c4ccc(c1)c2c34 ; CC1(C)c2ccccc2-c2ccc3c4ccccc4n(-c4ccc(-c5ccc6ccc7cccc8ccc5c6c78)cc4)c3c21 ; c1cc2ccc3ccc(-c4ccc(-n5c6ccccc6c6ccc7c8ccccc8sc7c65)cc4)c4ccc(c1)c2c34]

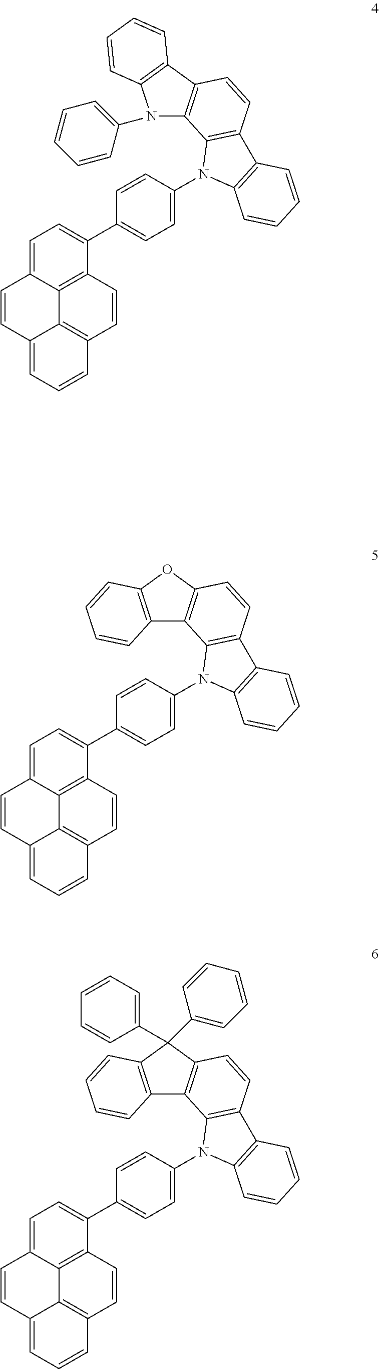 [SMILES: c1ccc(-n2c3ccccc3c3ccc4c5ccccc5n(-c5ccc(-c6ccc7ccc8cccc9ccc6c7c89)cc5)c4c32)cc1 ; c1cc2ccc3ccc(-c4ccc(-n5c6ccccc6c6ccc7oc8ccccc8c7c65)cc4)c4ccc(c1)c2c34 ; c1ccc(C2(c3ccccc3)c3ccccc3-c3c2ccc2c4ccccc4n(-c4ccc(-c5ccc6ccc7cccc8ccc5c6c78)cc4)c32)cc1]

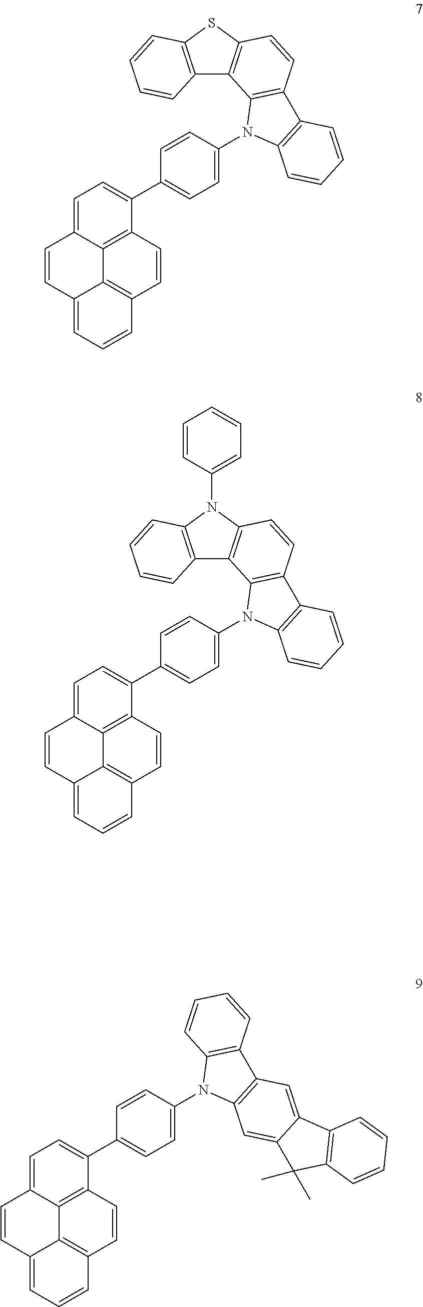 [SMILES: c1cc2ccc3ccc(-c4ccc(-n5c6ccccc6c6ccc7sc8ccccc8c7c65)cc4)c4ccc(c1)c2c34 ; c1ccc(-n2c3ccccc3c3c2ccc2c4ccccc4n(-c4ccc(-c5ccc6ccc7cccc8ccc5c6c78)cc4)c23)cc1 ; CC1(C)c2ccccc2-c2cc3c4ccccc4n(-c4ccc(-c5ccc6ccc7cccc8ccc5c6c78)cc4)c3cc21]

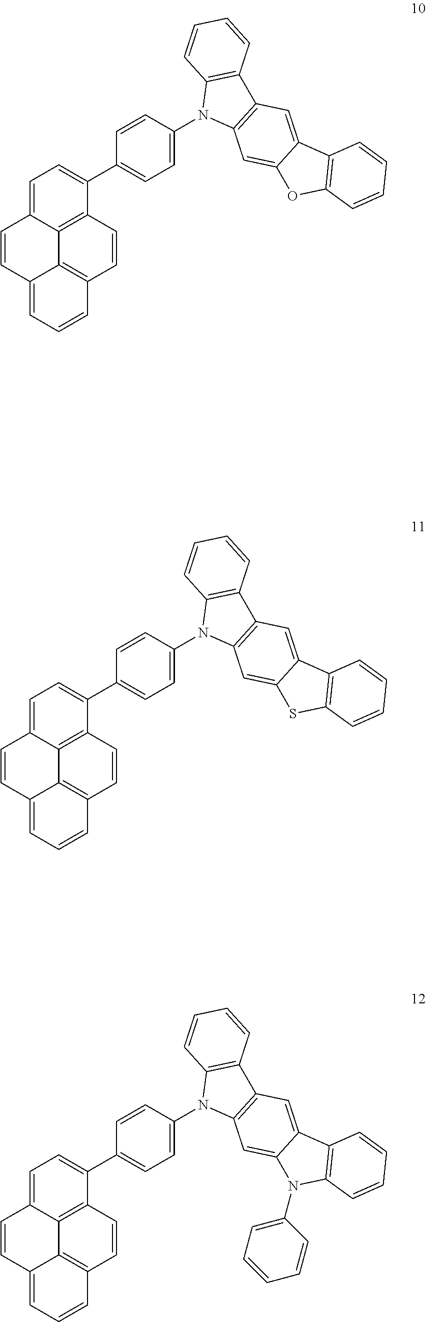 [SMILES: c1cc2ccc3ccc(-c4ccc(-n5c6ccccc6c6cc7c(cc65)oc5ccccc57)cc4)c4ccc(c1)c2c34 ; c1cc2ccc3ccc(-c4ccc(-n5c6ccccc6c6cc7c(cc65)sc5ccccc57)cc4)c4ccc(c1)c2c34 ; c1ccc(-n2c3ccccc3c3cc4c5ccccc5n(-c5ccc(-c6ccc7ccc8cccc9ccc6c7c89)cc5)c4cc32)cc1]

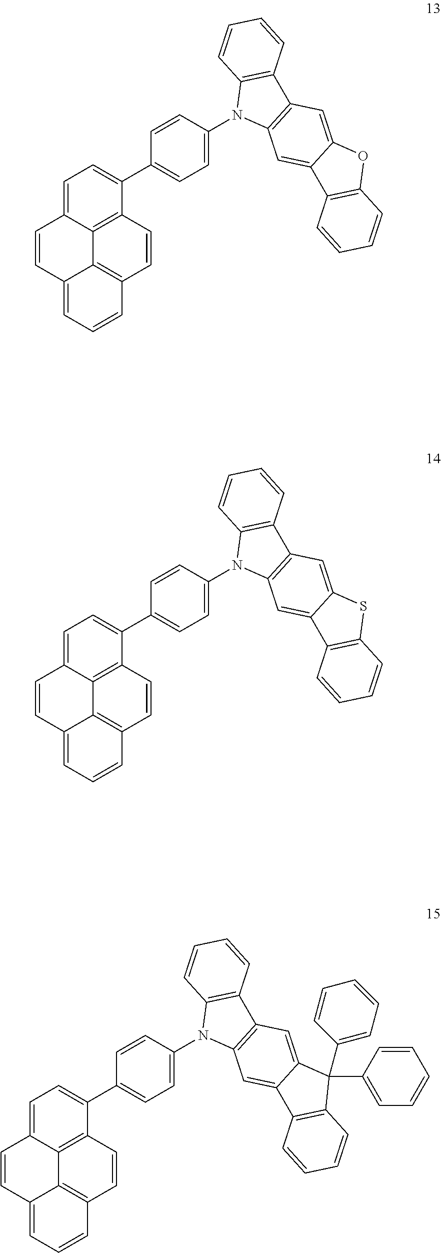 [SMILES: c1cc2ccc3ccc(-c4ccc(-n5c6ccccc6c6cc7oc8ccccc8c7cc65)cc4)c4ccc(c1)c2c34 ; c1cc2ccc3ccc(-c4ccc(-n5c6ccccc6c6cc7sc8ccccc8c7cc65)cc4)c4ccc(c1)c2c34 ; c1ccc(C2(c3ccccc3)c3ccccc3-c3cc4c(cc32)c2ccccc2n4-c2ccc(-c3ccc4ccc5cccc6ccc3c4c56)cc2)cc1]

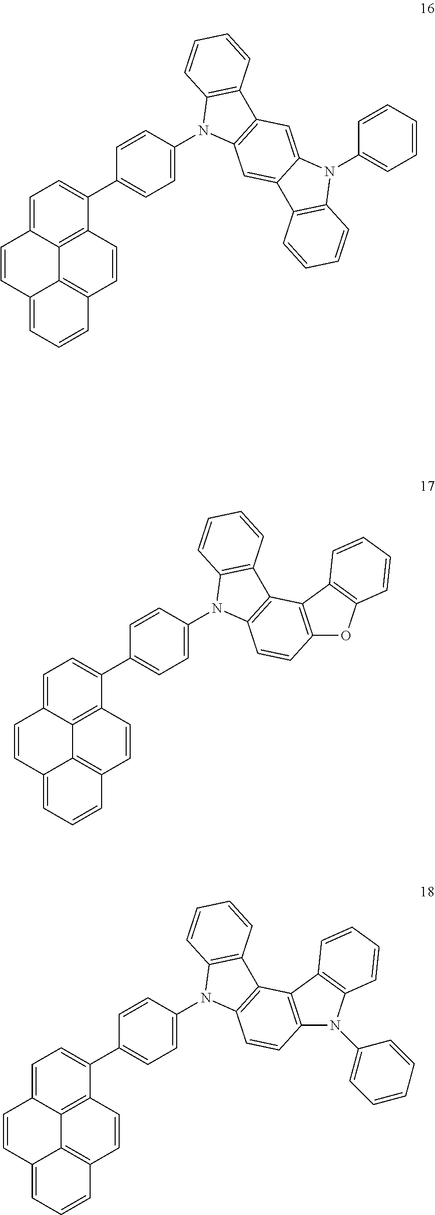 [SMILES: c1ccc(-n2c3ccccc3c3cc4c(cc32)c2ccccc2n4-c2ccc(-c3ccc4ccc5cccc6ccc3c4c56)cc2)cc1 ; c1cc2ccc3ccc(-c4ccc(-n5c6ccccc6c6c7c(ccc65)oc5ccccc57)cc4)c4ccc(c1)c2c34 ; c1ccc(-n2c3ccccc3c3c4c5ccccc5n(-c5ccc(-c6ccc7ccc8cccc9ccc6c7c89)cc5)c4ccc32)cc1]

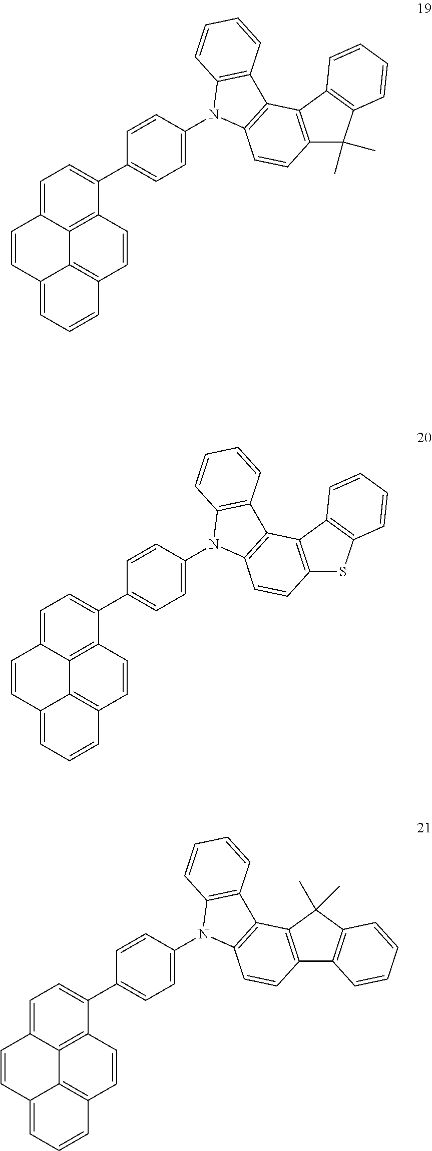 [SMILES: CC1(C)c2ccccc2-c2c1ccc1c2c2ccccc2n1-c1ccc(-c2ccc3ccc4cccc5ccc2c3c45)cc1 ; c1cc2ccc3ccc(-c4ccc(-n5c6ccccc6c6c7c(ccc65)sc5ccccc57)cc4)c4ccc(c1)c2c34 ; CC1(C)c2ccccc2-c2ccc3c(c21)c1ccccc1n3-c1ccc(-c2ccc3ccc4cccc5ccc2c3c45)cc1]

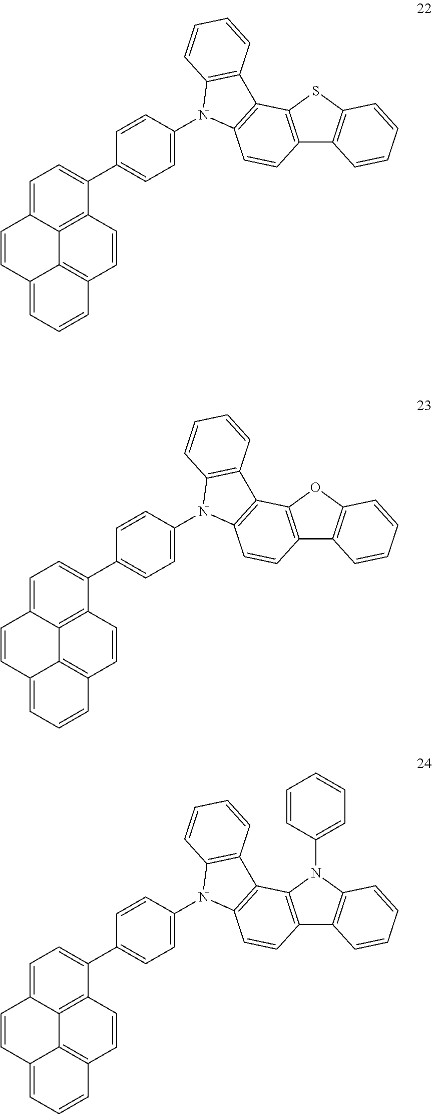 [SMILES: c1cc2ccc3ccc(-c4ccc(-n5c6ccccc6c6c7sc8ccccc8c7ccc65)cc4)c4ccc(c1)c2c34 ; c1cc2ccc3ccc(-c4ccc(-n5c6ccccc6c6c7oc8ccccc8c7ccc65)cc4)c4ccc(c1)c2c34 ; c1ccc(-n2c3ccccc3c3ccc4c(c5ccccc5n4-c4ccc(-c5ccc6ccc7cccc8ccc5c6c78)cc4)c32)cc1]

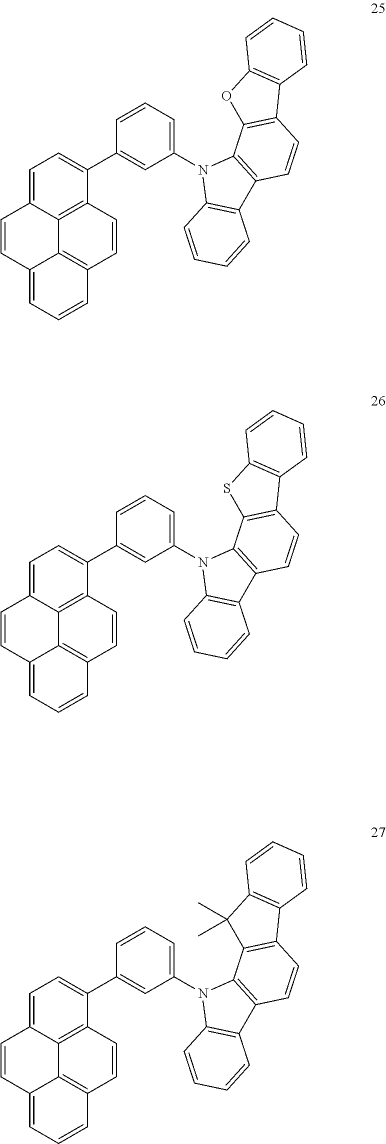 [SMILES: c1cc(-c2ccc3ccc4cccc5ccc2c3c45)cc(-n2c3ccccc3c3ccc4c5ccccc5oc4c32)c1 ; c1cc(-c2ccc3ccc4cccc5ccc2c3c45)cc(-n2c3ccccc3c3ccc4c5ccccc5sc4c32)c1 ; CC1(C)c2ccccc2-c2ccc3c4ccccc4n(-c4cccc(-c5ccc6ccc7cccc8ccc5c6c78)c4)c3c21]

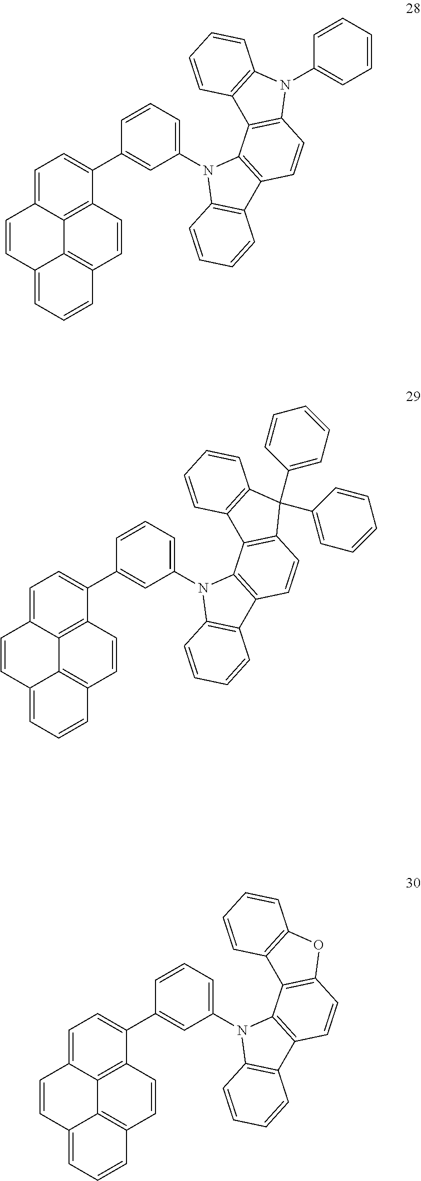 [SMILES: c1ccc(-n2c3ccccc3c3c2ccc2c4ccccc4n(-c4cccc(-c5ccc6ccc7cccc8ccc5c6c78)c4)c23)cc1 ; c1ccc(C2(c3ccccc3)c3ccccc3-c3c2ccc2c4ccccc4n(-c4cccc(-c5ccc6ccc7cccc8ccc5c6c78)c4)c32)cc1 ; c1cc(-c2ccc3ccc4cccc5ccc2c3c45)cc(-n2c3ccccc3c3ccc4oc5ccccc5c4c32)c1]

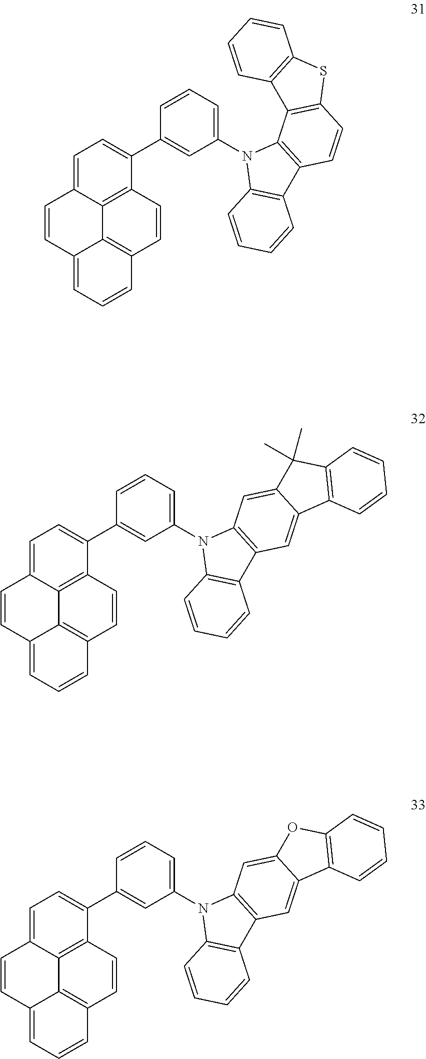 [SMILES: c1cc(-c2ccc3ccc4cccc5ccc2c3c45)cc(-n2c3ccccc3c3ccc4sc5ccccc5c4c32)c1 ; CC1(C)c2ccccc2-c2cc3c4ccccc4n(-c4cccc(-c5ccc6ccc7cccc8ccc5c6c78)c4)c3cc21 ; c1cc(-c2ccc3ccc4cccc5ccc2c3c45)cc(-n2c3ccccc3c3cc4c(cc32)oc2ccccc24)c1]

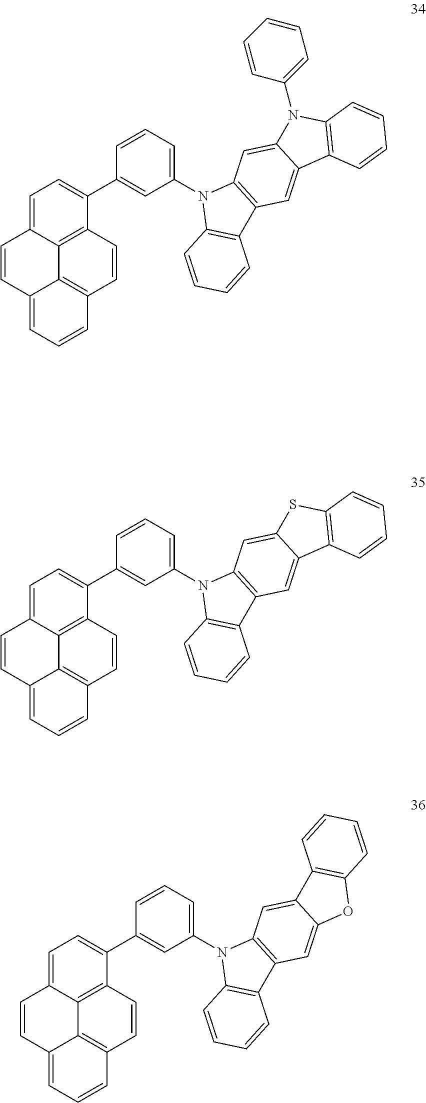 [SMILES: c1ccc(-n2c3ccccc3c3cc4c5ccccc5n(-c5cccc(-c6ccc7ccc8cccc9ccc6c7c89)c5)c4cc32)cc1 ; c1cc(-c2ccc3ccc4cccc5ccc2c3c45)cc(-n2c3ccccc3c3cc4c(cc32)sc2ccccc24)c1 ; c1cc(-c2ccc3ccc4cccc5ccc2c3c45)cc(-n2c3ccccc3c3cc4oc5ccccc5c4cc32)c1]

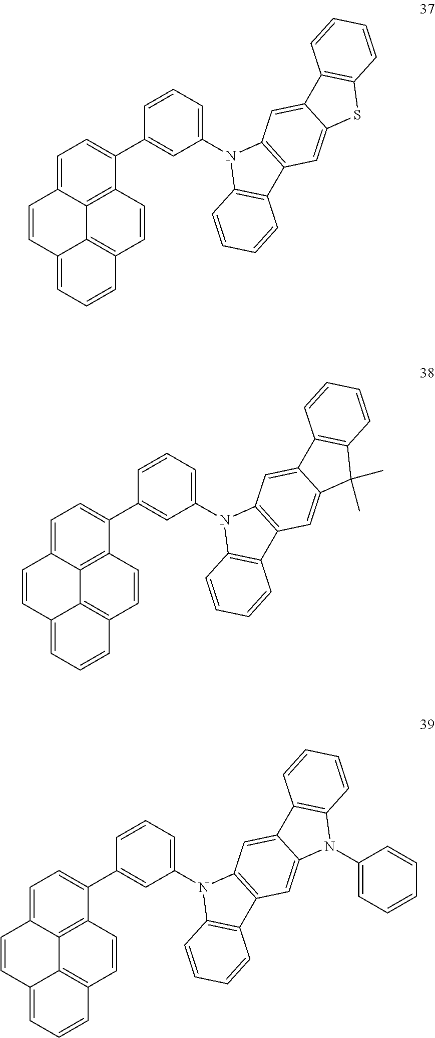 [SMILES: c1cc(-c2ccc3ccc4cccc5ccc2c3c45)cc(-n2c3ccccc3c3cc4sc5ccccc5c4cc32)c1 ; CC1(C)c2ccccc2-c2cc3c(cc21)c1ccccc1n3-c1cccc(-c2ccc3ccc4cccc5ccc2c3c45)c1 ; c1ccc(-n2c3ccccc3c3cc4c(cc32)c2ccccc2n4-c2cccc(-c3ccc4ccc5cccc6ccc3c4c56)c2)cc1]

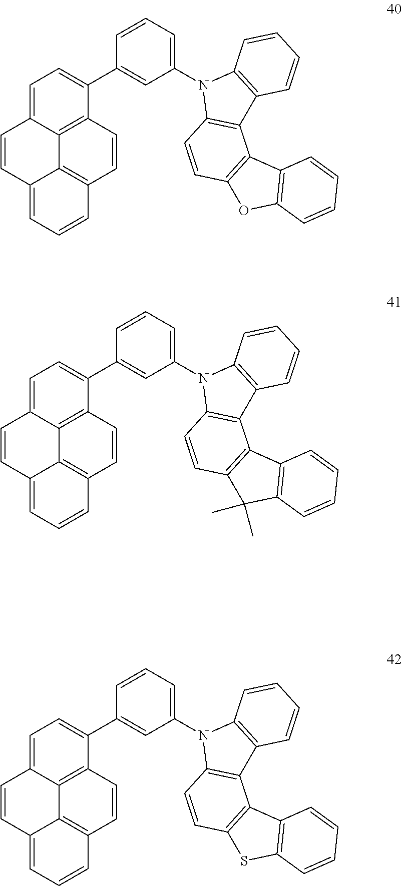 [SMILES: c1cc(-c2ccc3ccc4cccc5ccc2c3c45)cc(-n2c3ccccc3c3c4c(ccc32)oc2ccccc24)c1 ; CC1(C)c2ccccc2-c2c1ccc1c2c2ccccc2n1-c1cccc(-c2ccc3ccc4cccc5ccc2c3c45)c1 ; c1cc(-c2ccc3ccc4cccc5ccc2c3c45)cc(-n2c3ccccc3c3c4c(ccc32)sc2ccccc24)c1]

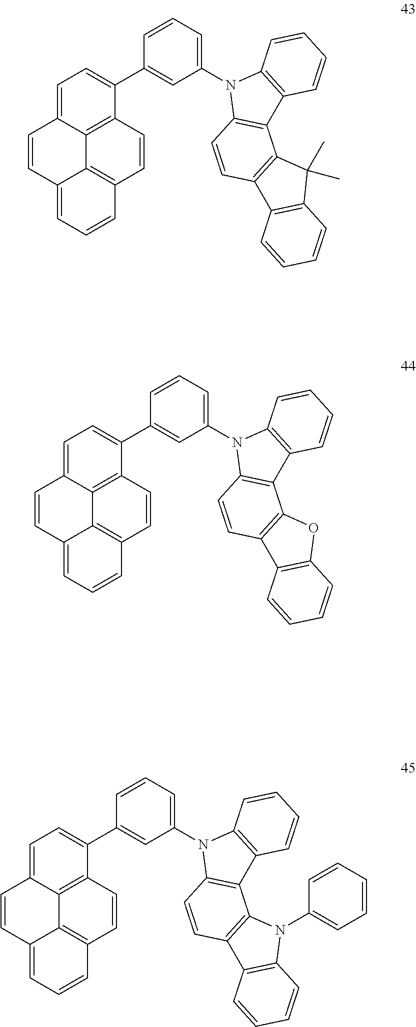 [SMILES: CC1(C)c2ccccc2-c2ccc3c(c21)c1ccccc1n3-c1cccc(-c2ccc3ccc4cccc5ccc2c3c45)c1 ; c1cc(-c2ccc3ccc4cccc5ccc2c3c45)cc(-n2c3ccccc3c3c4oc5ccccc5c4ccc32)c1 ; c1ccc(-n2c3ccccc3c3ccc4c(c5ccccc5n4-c4cccc(-c5ccc6ccc7cccc8ccc5c6c78)c4)c32)cc1]

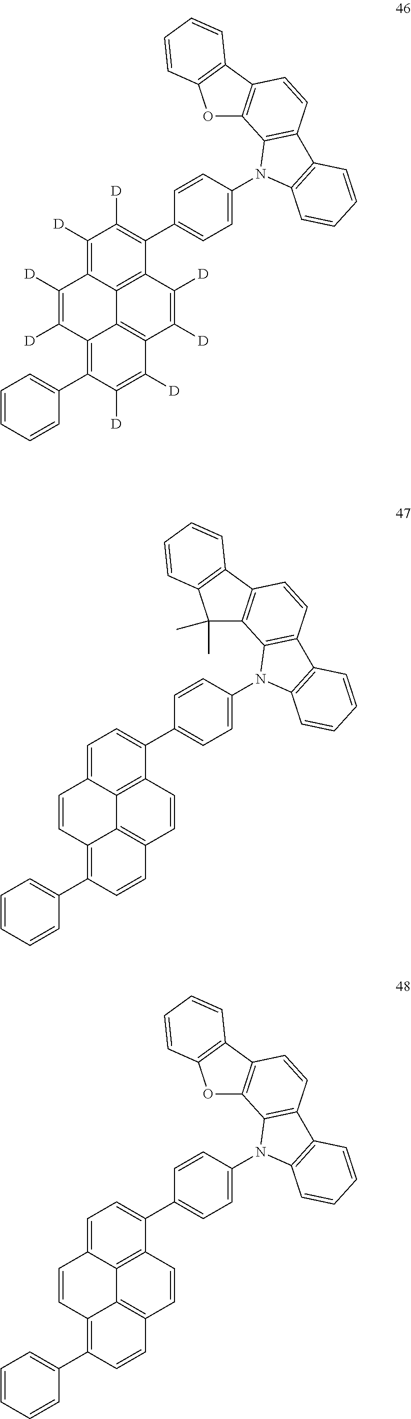 [SMILES: [2H]c1c([2H])c2c([2H])c([2H])c3c(-c4ccc(-n5c6ccccc6c6ccc7c8ccccc8oc7c65)cc4)c([2H])c([2H])c4c([2H])c([2H])c(c1-c1ccccc1)c2c43 ; CC1(C)c2ccccc2-c2ccc3c4ccccc4n(-c4ccc(-c5ccc6ccc7c(-c8ccccc8)ccc8ccc5c6c87)cc4)c3c21 ; c1ccc(-c2ccc3ccc4c(-c5ccc(-n6c7ccccc7c7ccc8c9ccccc9oc8c76)cc5)ccc5ccc2c3c54)cc1]

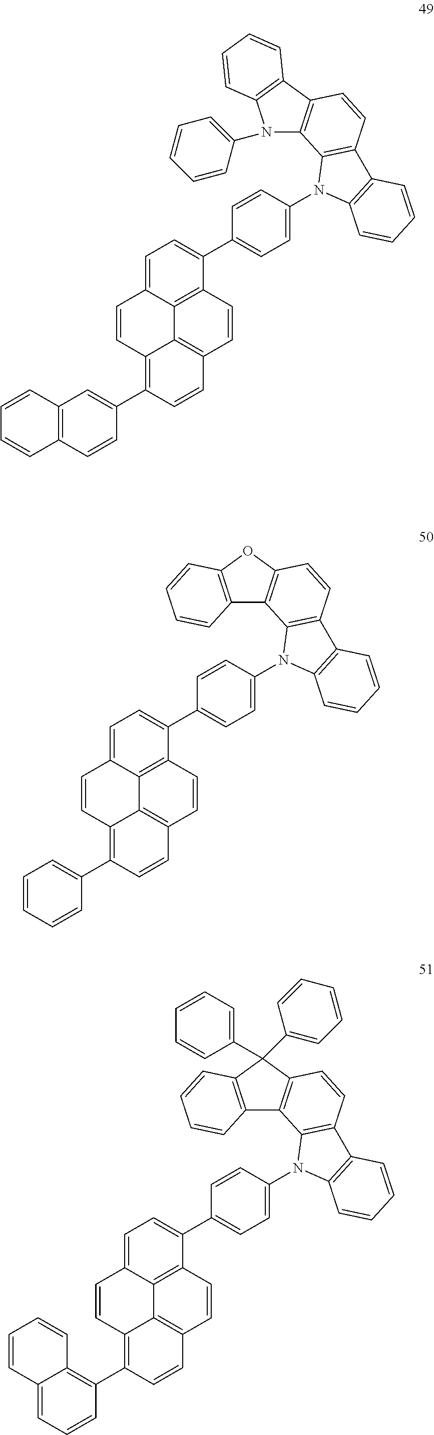 [SMILES: c1ccc(-n2c3ccccc3c3ccc4c5ccccc5n(-c5ccc(-c6ccc7ccc8c(-c9ccc%10ccccc%10c9)ccc9ccc6c7c98)cc5)c4c32)cc1 ; c1ccc(-c2ccc3ccc4c(-c5ccc(-n6c7ccccc7c7ccc8oc9ccccc9c8c76)cc5)ccc5ccc2c3c54)cc1 ; c1ccc(C2(c3ccccc3)c3ccccc3-c3c2ccc2c4ccccc4n(-c4ccc(-c5ccc6ccc7c(-c8cccc9ccccc89)ccc8ccc5c6c87)cc4)c32)cc1]

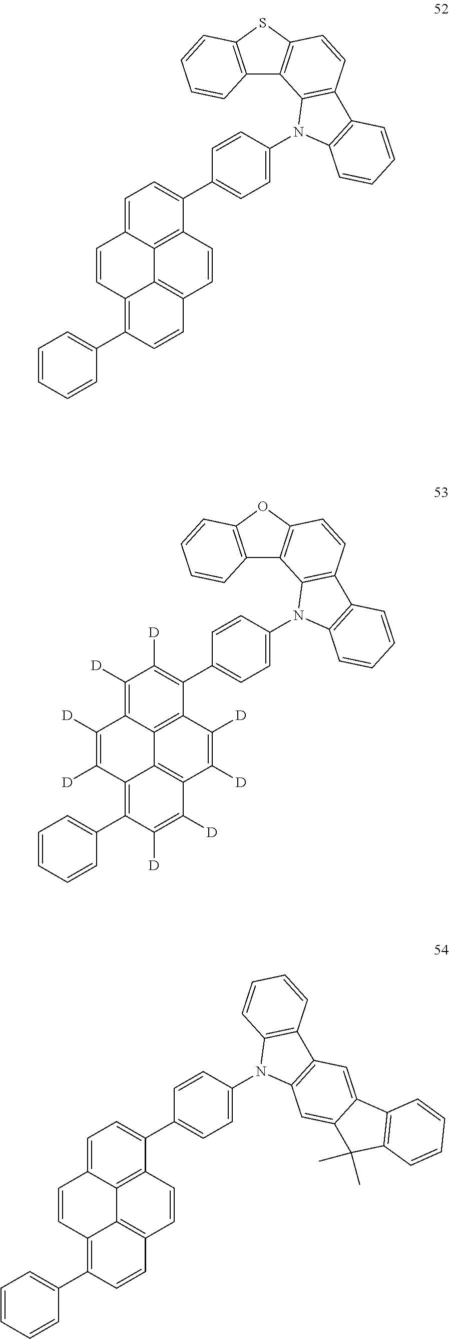 [SMILES: c1ccc(-c2ccc3ccc4c(-c5ccc(-n6c7ccccc7c7ccc8sc9ccccc9c8c76)cc5)ccc5ccc2c3c54)cc1 ; [2H]c1c([2H])c2c([2H])c([2H])c3c(-c4ccc(-n5c6ccccc6c6ccc7oc8ccccc8c7c65)cc4)c([2H])c([2H])c4c([2H])c([2H])c(c1-c1ccccc1)c2c43 ; CC1(C)c2ccccc2-c2cc3c4ccccc4n(-c4ccc(-c5ccc6ccc7c(-c8ccccc8)ccc8ccc5c6c87)cc4)c3cc21]

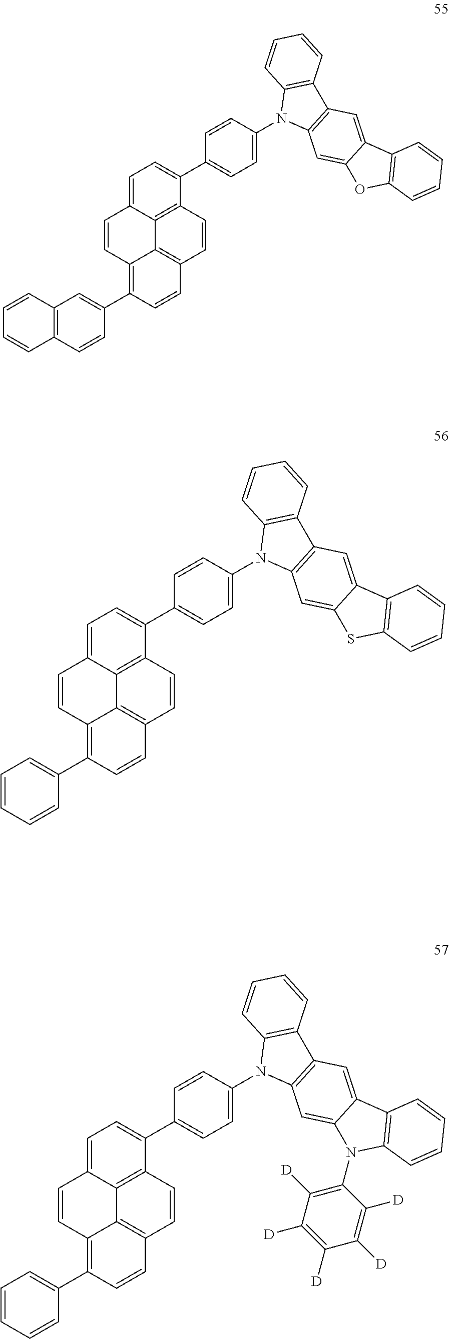 [SMILES: c1ccc2cc(-c3ccc4ccc5c(-c6ccc(-n7c8ccccc8c8cc9c(cc87)oc7ccccc79)cc6)ccc6ccc3c4c65)ccc2c1 ; c1ccc(-c2ccc3ccc4c(-c5ccc(-n6c7ccccc7c7cc8c(cc76)sc6ccccc68)cc5)ccc5ccc2c3c54)cc1 ; [2H]c1c([2H])c([2H])c(-n2c3ccccc3c3cc4c5ccccc5n(-c5ccc(-c6ccc7ccc8c(-c9ccccc9)ccc9ccc6c7c98)cc5)c4cc32)c([2H])c1[2H]]

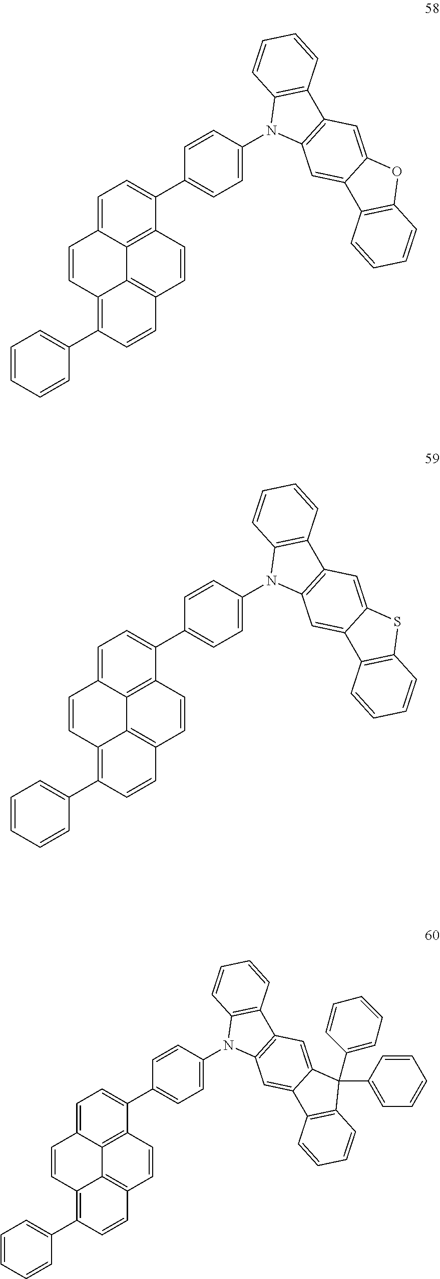 [SMILES: c1ccc(-c2ccc3ccc4c(-c5ccc(-n6c7ccccc7c7cc8oc9ccccc9c8cc76)cc5)ccc5ccc2c3c54)cc1 ; c1ccc(-c2ccc3ccc4c(-c5ccc(-n6c7ccccc7c7cc8sc9ccccc9c8cc76)cc5)ccc5ccc2c3c54)cc1 ; c1ccc(-c2ccc3ccc4c(-c5ccc(-n6c7ccccc7c7cc8c(cc76)-c6ccccc6C8(c6ccccc6)c6ccccc6)cc5)ccc5ccc2c3c54)cc1]

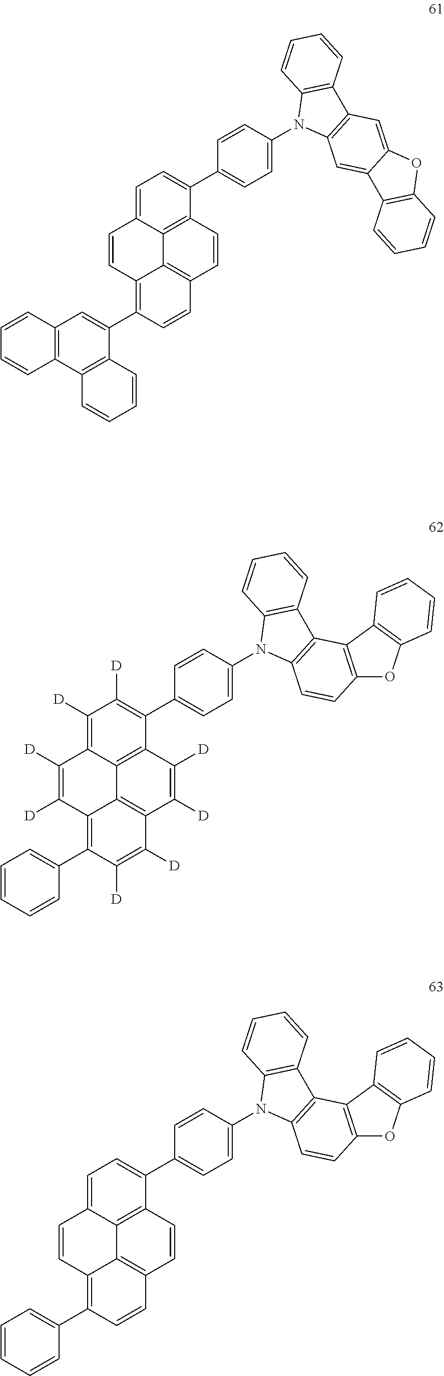 [SMILES: c1ccc2c(c1)cc(-c1ccc3ccc4c(-c5ccc(-n6c7ccccc7c7cc8oc9ccccc9c8cc76)cc5)ccc5ccc1c3c54)c1ccccc12 ; [2H]c1c([2H])c2c([2H])c([2H])c3c(-c4ccc(-n5c6ccccc6c6c7c(ccc65)oc5ccccc57)cc4)c([2H])c([2H])c4c([2H])c([2H])c(c1-c1ccccc1)c2c43 ; c1ccc(-c2ccc3ccc4c(-c5ccc(-n6c7ccccc7c7c8c(ccc76)oc6ccccc68)cc5)ccc5ccc2c3c54)cc1]

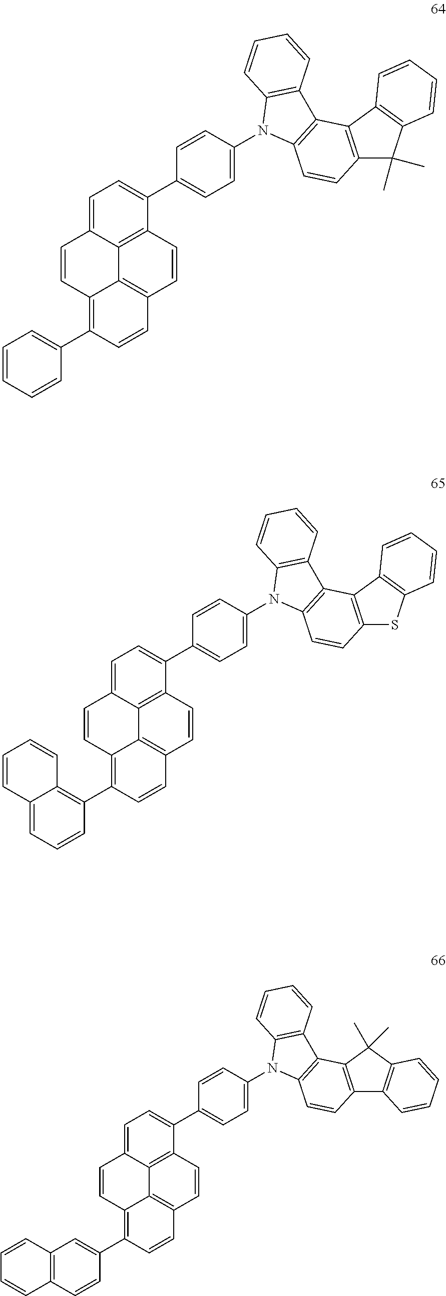 [SMILES: CC1(C)c2ccccc2-c2c1ccc1c2c2ccccc2n1-c1ccc(-c2ccc3ccc4c(-c5ccccc5)ccc5ccc2c3c54)cc1 ; c1ccc2c(-c3ccc4ccc5c(-c6ccc(-n7c8ccccc8c8c9c(ccc87)sc7ccccc79)cc6)ccc6ccc3c4c65)cccc2c1 ; CC1(C)c2ccccc2-c2ccc3c(c21)c1ccccc1n3-c1ccc(-c2ccc3ccc4c(-c5ccc6ccccc6c5)ccc5ccc2c3c54)cc1]

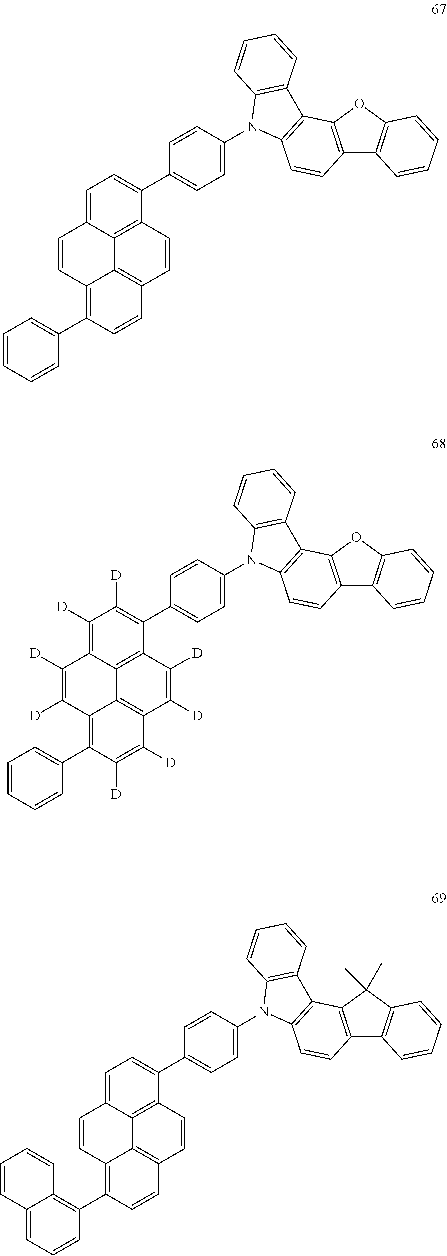 [SMILES: c1ccc(-c2ccc3ccc4c(-c5ccc(-n6c7ccccc7c7c8oc9ccccc9c8ccc76)cc5)ccc5ccc2c3c54)cc1 ; [2H]c1c([2H])c2c([2H])c([2H])c3c(-c4ccc(-n5c6ccccc6c6c7oc8ccccc8c7ccc65)cc4)c([2H])c([2H])c4c([2H])c([2H])c(c1-c1ccccc1)c2c43 ; CC1(C)c2ccccc2-c2ccc3c(c21)c1ccccc1n3-c1ccc(-c2ccc3ccc4c(-c5cccc6ccccc56)ccc5ccc2c3c54)cc1]

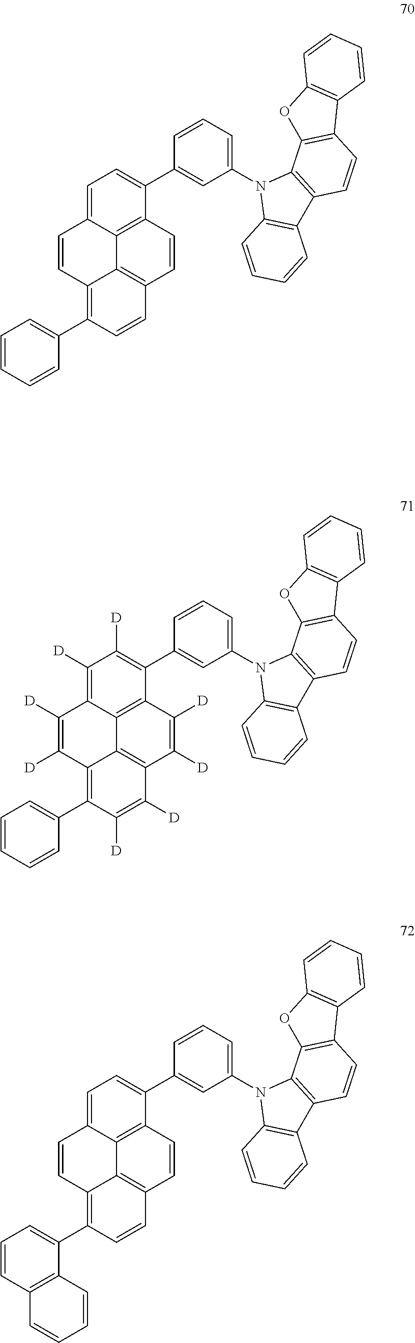 [SMILES: c1ccc(-c2ccc3ccc4c(-c5cccc(-n6c7ccccc7c7ccc8c9ccccc9oc8c76)c5)ccc5ccc2c3c54)cc1 ; [2H]c1c([2H])c2c([2H])c([2H])c3c(-c4cccc(-n5c6ccccc6c6ccc7c8ccccc8oc7c65)c4)c([2H])c([2H])c4c([2H])c([2H])c(c1-c1ccccc1)c2c43 ; c1cc(-c2ccc3ccc4c(-c5cccc6ccccc56)ccc5ccc2c3c54)cc(-n2c3ccccc3c3ccc4c5ccccc5oc4c32)c1]

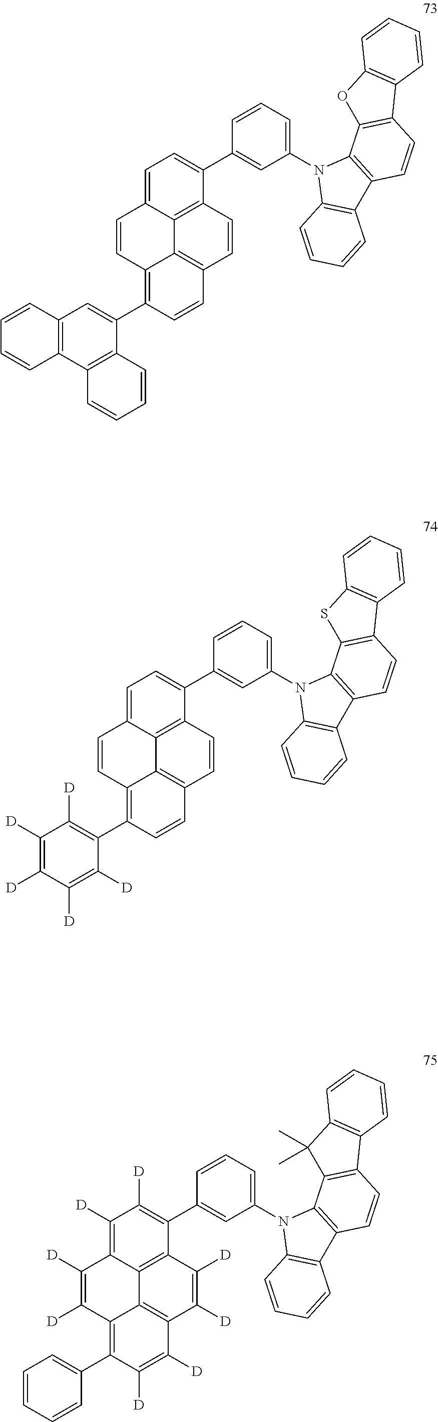 [SMILES: c1cc(-c2ccc3ccc4c(-c5cc6ccccc6c6ccccc56)ccc5ccc2c3c54)cc(-n2c3ccccc3c3ccc4c5ccccc5oc4c32)c1 ; [2H]c1c([2H])c([2H])c(-c2ccc3ccc4c(-c5cccc(-n6c7ccccc7c7ccc8c9ccccc9sc8c76)c5)ccc5ccc2c3c54)c([2H])c1[2H] ; [2H]c1c([2H])c2c([2H])c([2H])c3c(-c4cccc(-n5c6ccccc6c6ccc7c(c65)C(C)(C)c5ccccc5-7)c4)c([2H])c([2H])c4c([2H])c([2H])c(c1-c1ccccc1)c2c43]

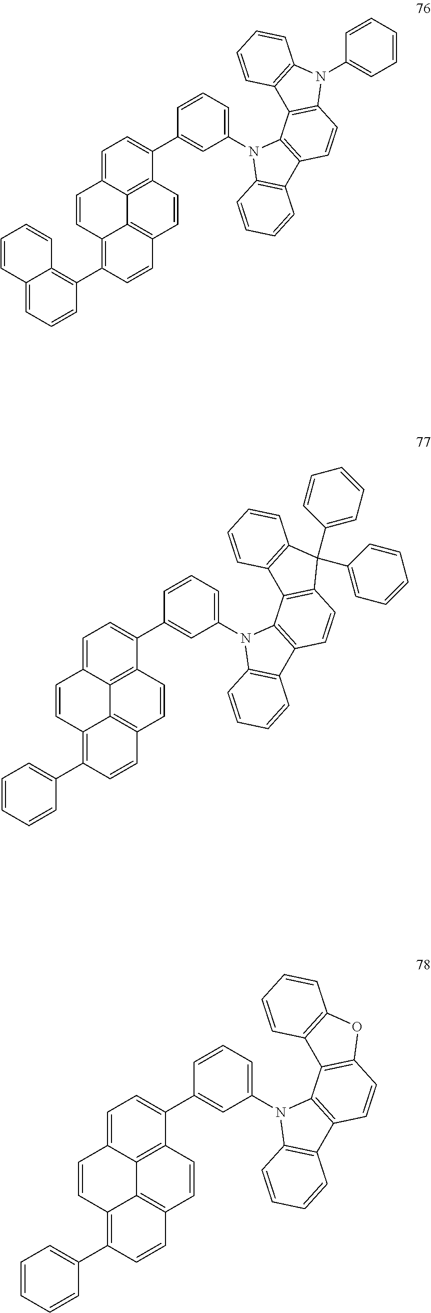 [SMILES: c1ccc(-n2c3ccccc3c3c2ccc2c4ccccc4n(-c4cccc(-c5ccc6ccc7c(-c8cccc9ccccc89)ccc8ccc5c6c87)c4)c23)cc1 ; c1ccc(-c2ccc3ccc4c(-c5cccc(-n6c7ccccc7c7ccc8c(c76)-c6ccccc6C8(c6ccccc6)c6ccccc6)c5)ccc5ccc2c3c54)cc1 ; c1ccc(-c2ccc3ccc4c(-c5cccc(-n6c7ccccc7c7ccc8oc9ccccc9c8c76)c5)ccc5ccc2c3c54)cc1]

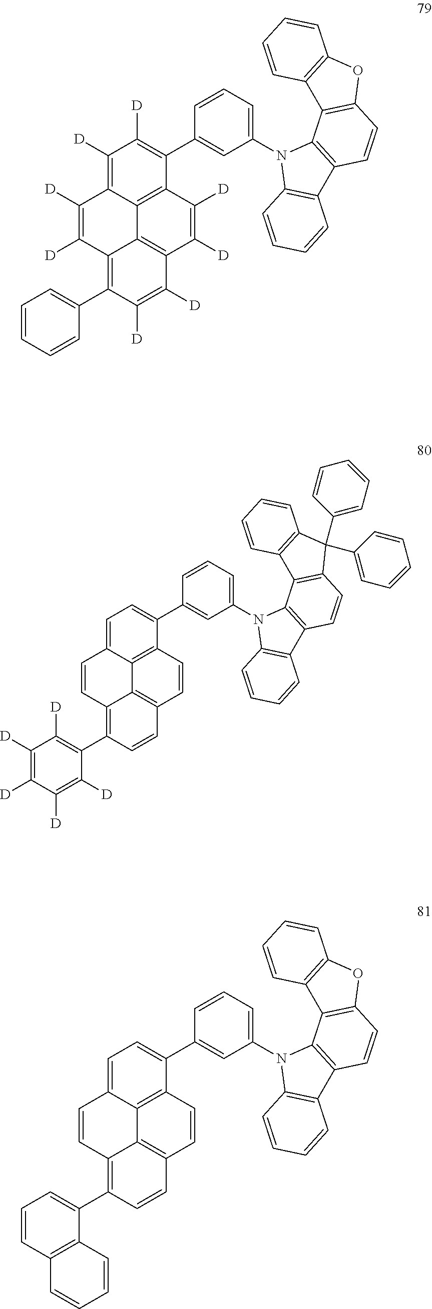 [SMILES: [2H]c1c([2H])c2c([2H])c([2H])c3c(-c4cccc(-n5c6ccccc6c6ccc7oc8ccccc8c7c65)c4)c([2H])c([2H])c4c([2H])c([2H])c(c1-c1ccccc1)c2c43 ; [2H]c1c([2H])c([2H])c(-c2ccc3ccc4c(-c5cccc(-n6c7ccccc7c7ccc8c(c76)-c6ccccc6C8(c6ccccc6)c6ccccc6)c5)ccc5ccc2c3c54)c([2H])c1[2H] ; c1cc(-c2ccc3ccc4c(-c5cccc6ccccc56)ccc5ccc2c3c54)cc(-n2c3ccccc3c3ccc4oc5ccccc5c4c32)c1]

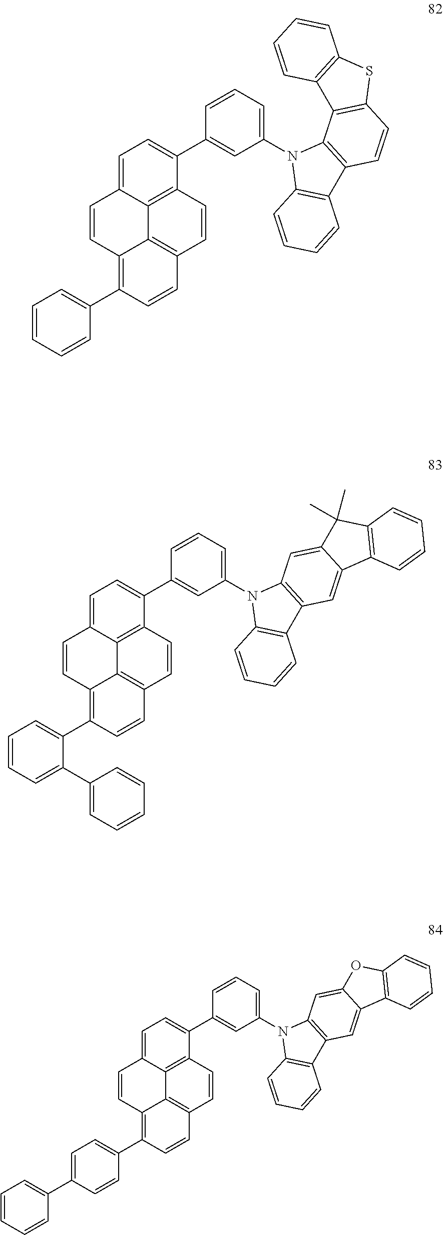 [SMILES: c1ccc(-c2ccc3ccc4c(-c5cccc(-n6c7ccccc7c7ccc8sc9ccccc9c8c76)c5)ccc5ccc2c3c54)cc1 ; CC1(C)c2ccccc2-c2cc3c4ccccc4n(-c4cccc(-c5ccc6ccc7c(-c8ccccc8-c8ccccc8)ccc8ccc5c6c87)c4)c3cc21 ; c1ccc(-c2ccc(-c3ccc4ccc5c(-c6cccc(-n7c8ccccc8c8cc9c(cc87)oc7ccccc79)c6)ccc6ccc3c4c65)cc2)cc1]

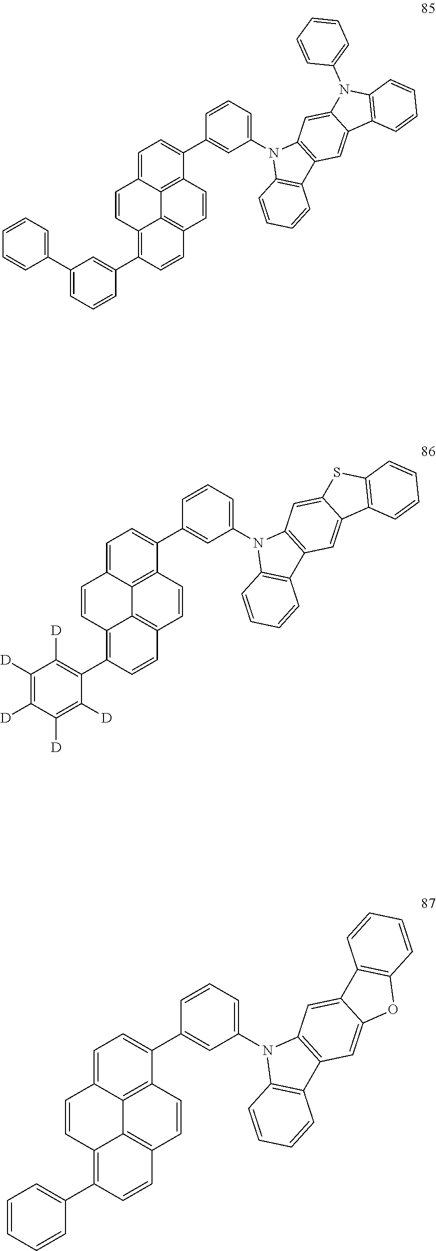 [SMILES: c1ccc(-c2cccc(-c3ccc4ccc5c(-c6cccc(-n7c8ccccc8c8cc9c%10ccccc%10n(-c%10ccccc%10)c9cc87)c6)ccc6ccc3c4c65)c2)cc1 ; [2H]c1c([2H])c([2H])c(-c2ccc3ccc4c(-c5cccc(-n6c7ccccc7c7cc8c(cc76)sc6ccccc68)c5)ccc5ccc2c3c54)c([2H])c1[2H] ; c1ccc(-c2ccc3ccc4c(-c5cccc(-n6c7ccccc7c7cc8oc9ccccc9c8cc76)c5)ccc5ccc2c3c54)cc1]

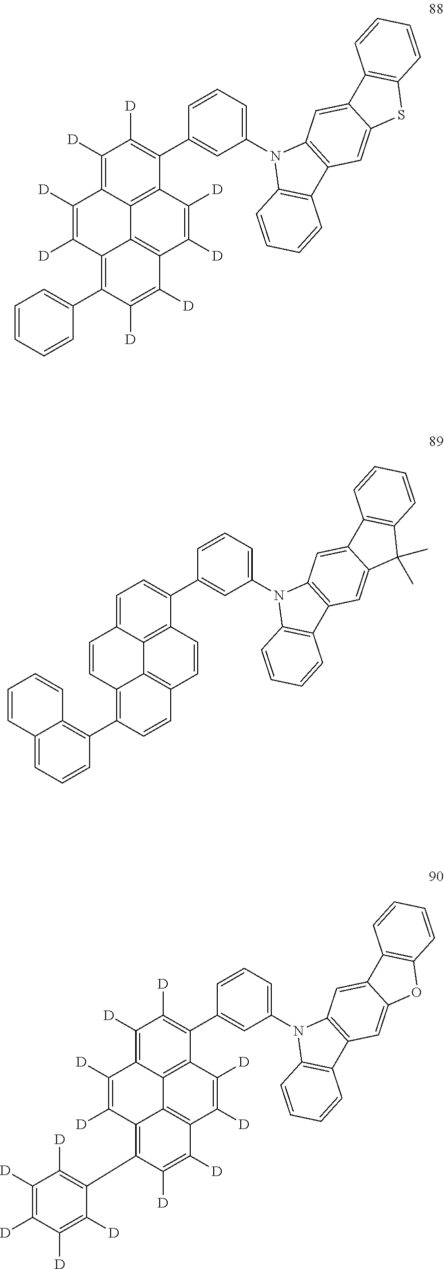 [SMILES: [2H]c1c([2H])c2c([2H])c([2H])c3c(-c4cccc(-n5c6ccccc6c6cc7sc8ccccc8c7cc65)c4)c([2H])c([2H])c4c([2H])c([2H])c(c1-c1ccccc1)c2c43 ; CC1(C)c2ccccc2-c2cc3c(cc21)c1ccccc1n3-c1cccc(-c2ccc3ccc4c(-c5cccc6ccccc56)ccc5ccc2c3c54)c1 ; [2H]c1c([2H])c([2H])c(-c2c([2H])c([2H])c3c([2H])c([2H])c4c(-c5cccc(-n6c7ccccc7c7cc8oc9ccccc9c8cc76)c5)c([2H])c([2H])c5c([2H])c([2H])c2c3c54)c([2H])c1[2H]]

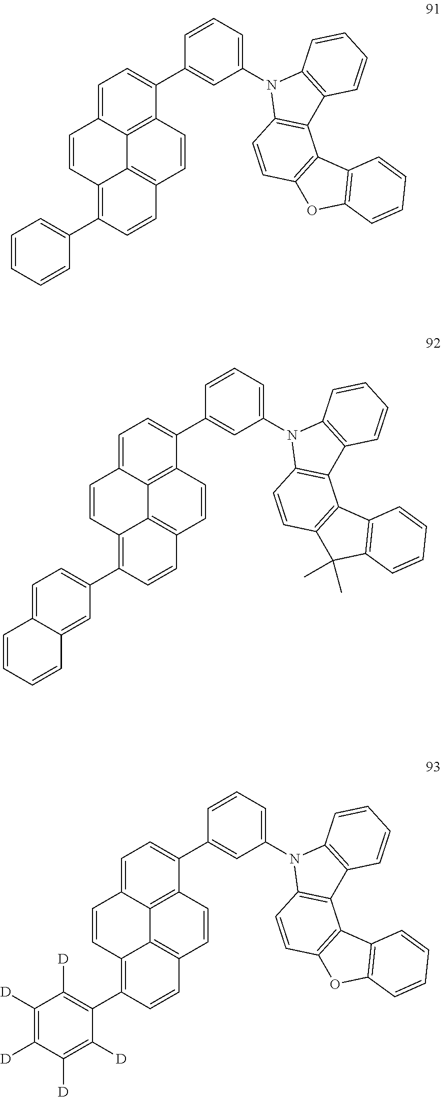 [SMILES: c1ccc(-c2ccc3ccc4c(-c5cccc(-n6c7ccccc7c7c8c(ccc76)oc6ccccc68)c5)ccc5ccc2c3c54)cc1 ; CC1(C)c2ccccc2-c2c1ccc1c2c2ccccc2n1-c1cccc(-c2ccc3ccc4c(-c5ccc6ccccc6c5)ccc5ccc2c3c54)c1 ; [2H]c1c([2H])c([2H])c(-c2ccc3ccc4c(-c5cccc(-n6c7ccccc7c7c8c(ccc76)oc6ccccc68)c5)ccc5ccc2c3c54)c([2H])c1[2H]]

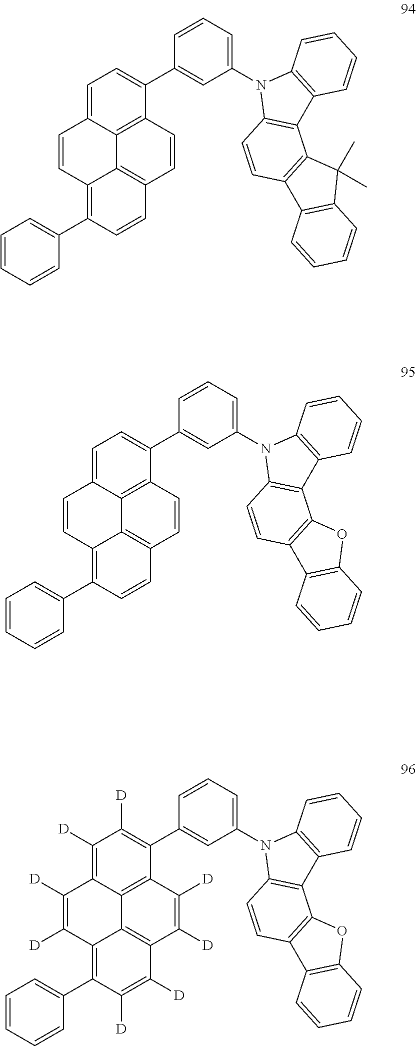 [SMILES: CC1(C)c2ccccc2-c2ccc3c(c21)c1ccccc1n3-c1cccc(-c2ccc3ccc4c(-c5ccccc5)ccc5ccc2c3c54)c1 ; c1ccc(-c2ccc3ccc4c(-c5cccc(-n6c7ccccc7c7c8oc9ccccc9c8ccc76)c5)ccc5ccc2c3c54)cc1 ; [2H]c1c([2H])c2c([2H])c([2H])c3c(-c4cccc(-n5c6ccccc6c6c7oc8ccccc8c7ccc65)c4)c([2H])c([2H])c4c([2H])c([2H])c(c1-c1ccccc1)c2c43]

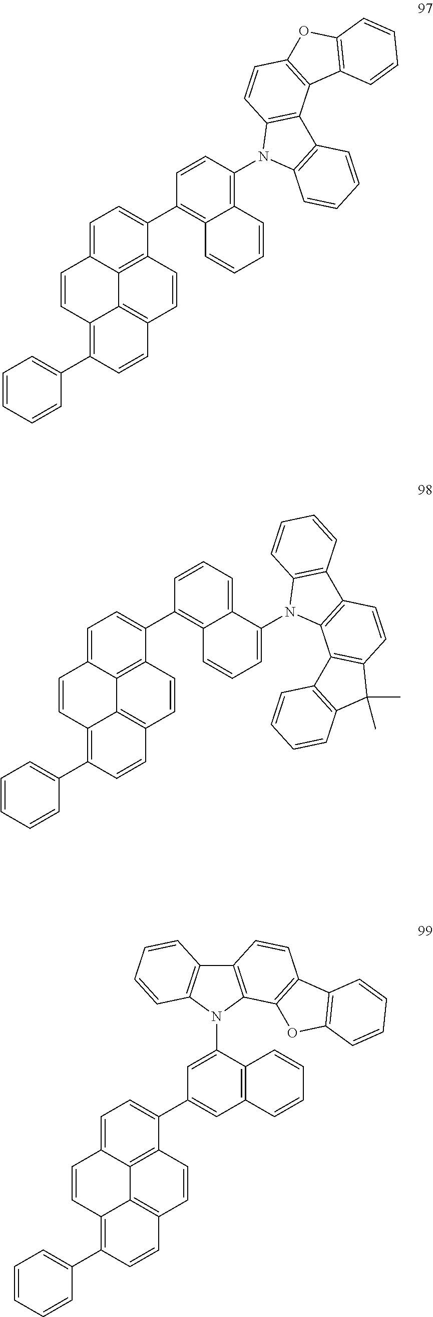 [SMILES: c1ccc(-c2ccc3ccc4c(-c5ccc(-n6c7ccccc7c7c8c(ccc76)oc6ccccc68)c6ccccc56)ccc5ccc2c3c54)cc1 ; CC1(C)c2ccccc2-c2c1ccc1c3ccccc3n(-c3cccc4c(-c5ccc6ccc7c(-c8ccccc8)ccc8ccc5c6c87)cccc34)c21 ; c1ccc(-c2ccc3ccc4c(-c5cc(-n6c7ccccc7c7ccc8c9ccccc9oc8c76)c6ccccc6c5)ccc5ccc2c3c54)cc1]

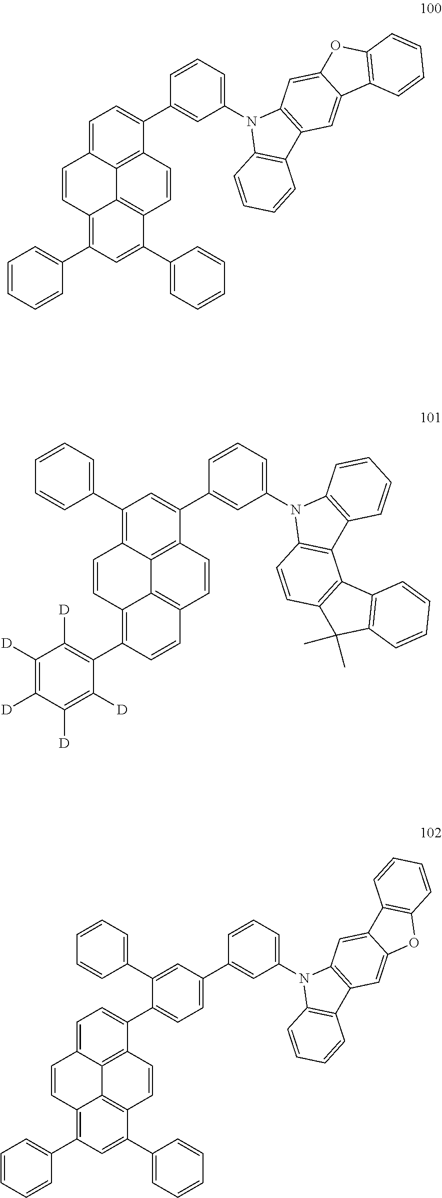 [SMILES: c1ccc(-c2cc(-c3ccccc3)c3ccc4c(-c5cccc(-n6c7ccccc7c7cc8c(cc76)oc6ccccc68)c5)ccc5ccc2c3c54)cc1 ; [2H]c1c([2H])c([2H])c(-c2ccc3ccc4c(-c5cccc(-n6c7ccccc7c7c8c(ccc76)C(C)(C)c6ccccc6-8)c5)cc(-c5ccccc5)c5ccc2c3c54)c([2H])c1[2H] ; c1ccc(-c2cc(-c3cccc(-n4c5ccccc5c5cc6oc7ccccc7c6cc54)c3)ccc2-c2ccc3ccc4c(-c5ccccc5)cc(-c5ccccc5)c5ccc2c3c45)cc1]

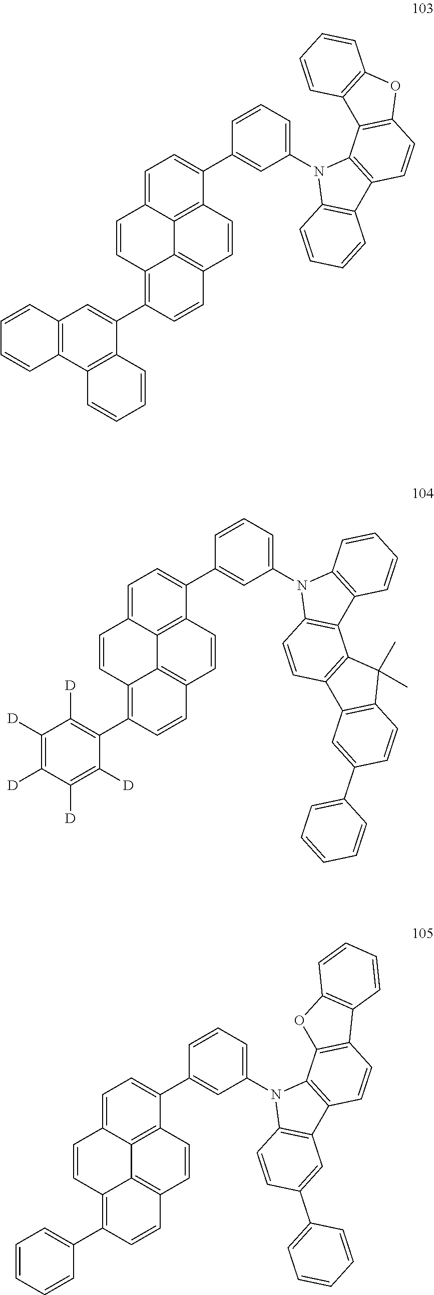 [SMILES: c1cc(-c2ccc3ccc4c(-c5cc6ccccc6c6ccccc56)ccc5ccc2c3c54)cc(-n2c3ccccc3c3ccc4oc5ccccc5c4c32)c1 ; [2H]c1c([2H])c([2H])c(-c2ccc3ccc4c(-c5cccc(-n6c7ccccc7c7c8c(ccc76)-c6cc(-c7ccccc7)ccc6C8(C)C)c5)ccc5ccc2c3c54)c([2H])c1[2H] ; c1ccc(-c2ccc3c(c2)c2ccc4c5ccccc5oc4c2n3-c2cccc(-c3ccc4ccc5c(-c6ccccc6)ccc6ccc3c4c65)c2)cc1]

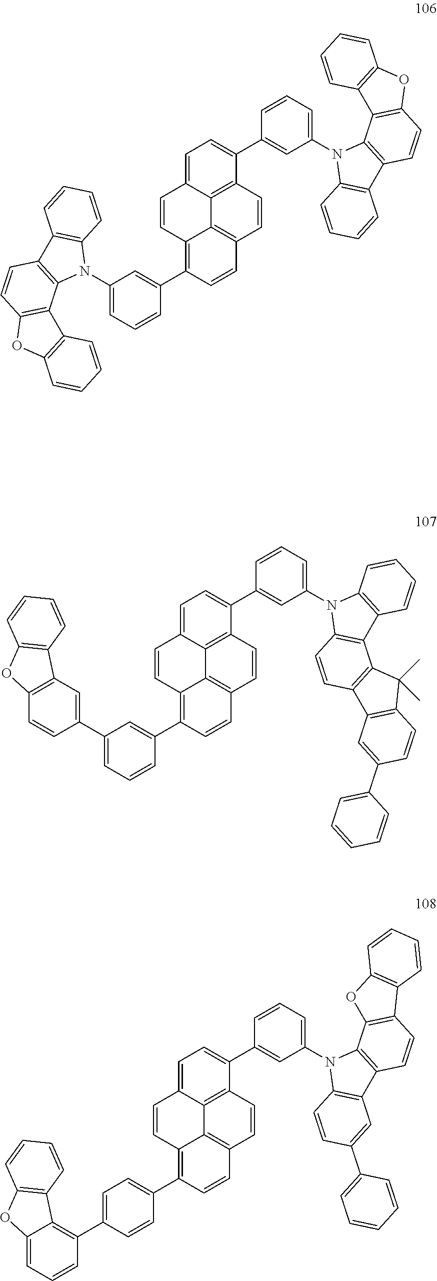 [SMILES: c1cc(-c2ccc3ccc4c(-c5cccc(-n6c7ccccc7c7ccc8oc9ccccc9c8c76)c5)ccc5ccc2c3c54)cc(-n2c3ccccc3c3ccc4oc5ccccc5c4c32)c1 ; CC1(C)c2ccc(-c3ccccc3)cc2-c2ccc3c(c21)c1ccccc1n3-c1cccc(-c2ccc3ccc4c(-c5cccc(-c6ccc7oc8ccccc8c7c6)c5)ccc5ccc2c3c54)c1 ; c1ccc(-c2ccc3c(c2)c2ccc4c5ccccc5oc4c2n3-c2cccc(-c3ccc4ccc5c(-c6ccc(-c7cccc8oc9ccccc9c78)cc6)ccc6ccc3c4c65)c2)cc1]

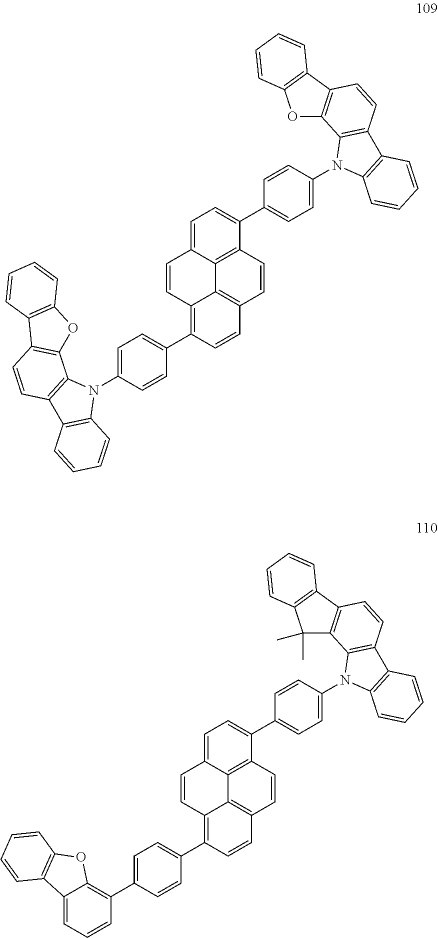 [SMILES: c1ccc2c(c1)oc1c2ccc2c3ccccc3n(-c3ccc(-c4ccc5ccc6c(-c7ccc(-n8c9ccccc9c9ccc%10c%11ccccc%11oc%10c98)cc7)ccc7ccc4c5c76)cc3)c21 ; CC1(C)c2ccccc2-c2ccc3c4ccccc4n(-c4ccc(-c5ccc6ccc7c(-c8ccc(-c9cccc%10c9oc9ccccc9%10)cc8)ccc8ccc5c6c87)cc4)c3c21]

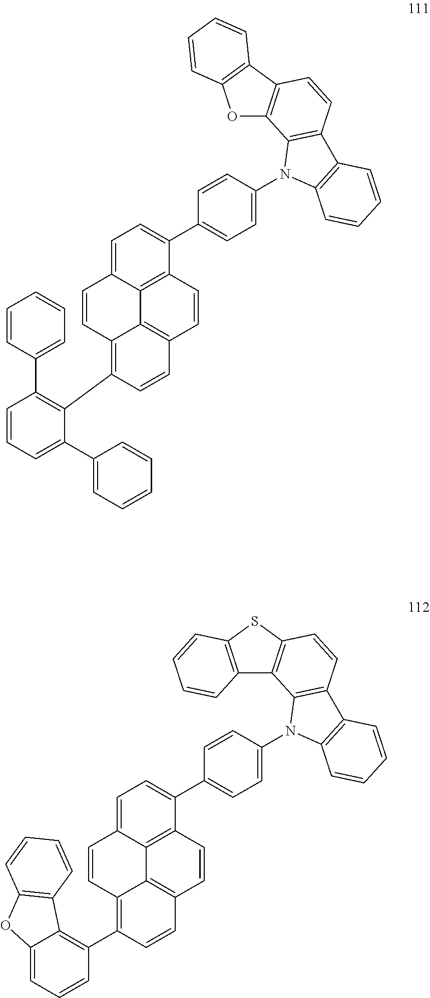 [SMILES: c1ccc(-c2cccc(-c3ccccc3)c2-c2ccc3ccc4c(-c5ccc(-n6c7ccccc7c7ccc8c9ccccc9oc8c76)cc5)ccc5ccc2c3c54)cc1 ; c1ccc2c(c1)oc1cccc(-c3ccc4ccc5c(-c6ccc(-n7c8ccccc8c8ccc9sc%10ccccc%10c9c87)cc6)ccc6ccc3c4c65)c12]

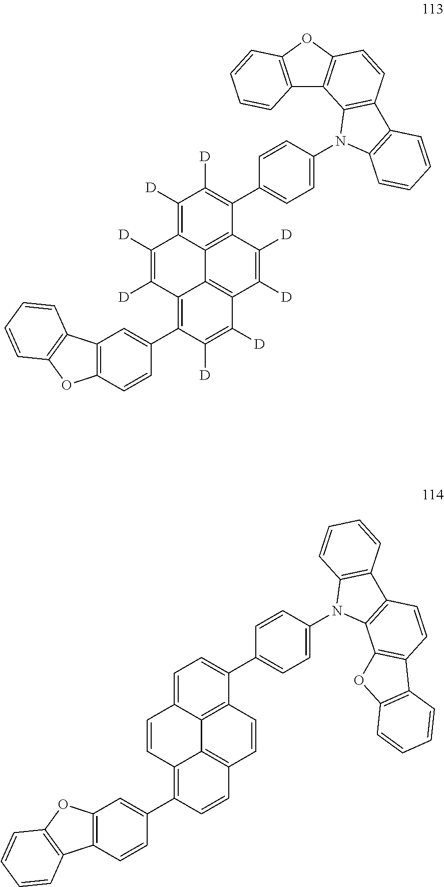 [SMILES: [2H]c1c([2H])c2c([2H])c([2H])c3c(-c4ccc5oc6ccccc6c5c4)c([2H])c([2H])c4c([2H])c([2H])c(c1-c1ccc(-n5c6ccccc6c6ccc7oc8ccccc8c7c65)cc1)c2c43 ; c1ccc2c(c1)oc1cc(-c3ccc4ccc5c(-c6ccc(-n7c8ccccc8c8ccc9c%10ccccc%10oc9c87)cc6)ccc6ccc3c4c65)ccc12]

According to one embodiment of the present invention, the organic compound represented by Formula 2 may be selected from the following compounds [201] to [245].

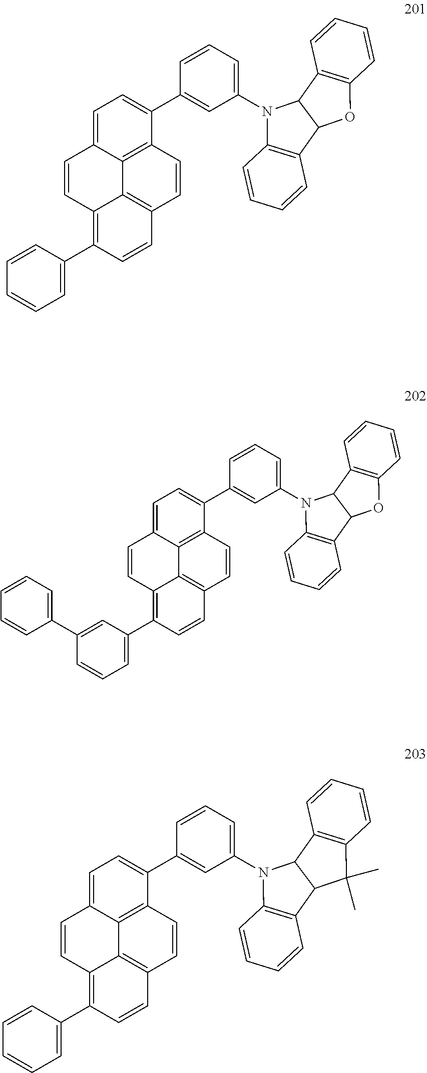 [SMILES: c1ccc(-c2ccc3ccc4c(-c5cccc(N6c7ccccc7C7Oc8ccccc8C76)c5)ccc5ccc2c3c54)cc1 ; c1ccc(-c2cccc(-c3ccc4ccc5c(-c6cccc(N7c8ccccc8C8Oc9ccccc9C87)c6)ccc6ccc3c4c65)c2)cc1 ; CC1(C)c2ccccc2C2C1c1ccccc1N2c1cccc(-c2ccc3ccc4c(-c5ccccc5)ccc5ccc2c3c54)c1]

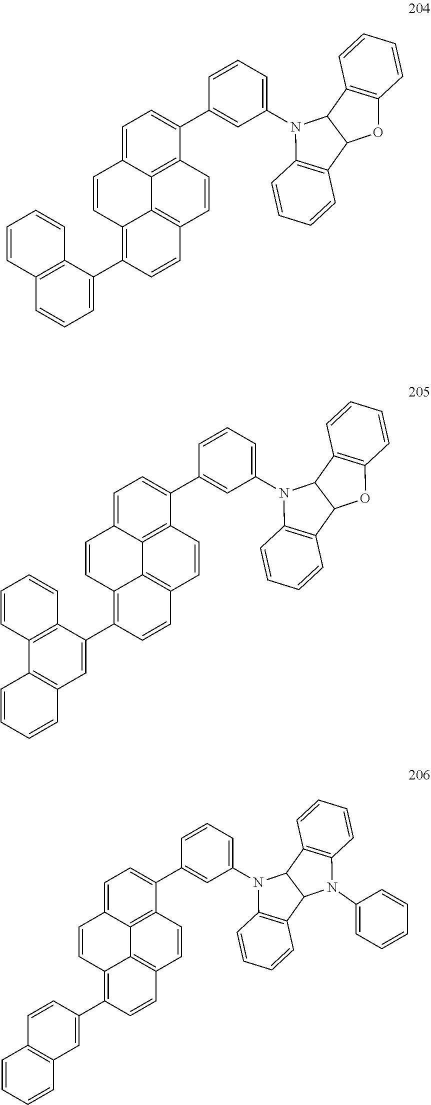 [SMILES: c1cc(-c2ccc3ccc4c(-c5cccc6ccccc56)ccc5ccc2c3c54)cc(N2c3ccccc3C3Oc4ccccc4C32)c1 ; c1cc(-c2ccc3ccc4c(-c5cc6ccccc6c6ccccc56)ccc5ccc2c3c54)cc(N2c3ccccc3C3Oc4ccccc4C32)c1 ; c1ccc(N2c3ccccc3C3C2c2ccccc2N3c2cccc(-c3ccc4ccc5c(-c6ccc7ccccc7c6)ccc6ccc3c4c65)c2)cc1]

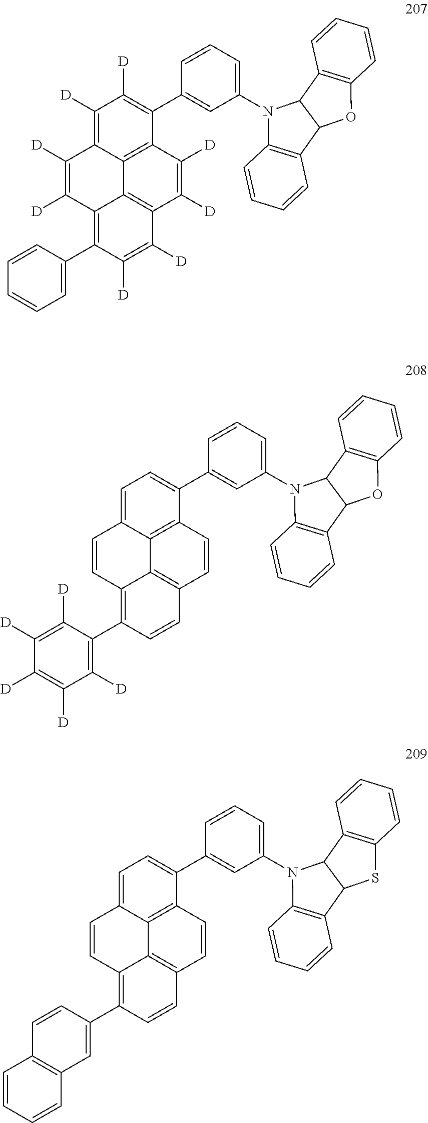 [SMILES: [2H]c1c([2H])c2c([2H])c([2H])c3c(-c4cccc(N5c6ccccc6C6Oc7ccccc7C65)c4)c([2H])c([2H])c4c([2H])c([2H])c(c1-c1ccccc1)c2c43 ; [2H]c1c([2H])c([2H])c(-c2ccc3ccc4c(-c5cccc(N6c7ccccc7C7Oc8ccccc8C76)c5)ccc5ccc2c3c54)c([2H])c1[2H] ; c1cc(-c2ccc3ccc4c(-c5ccc6ccccc6c5)ccc5ccc2c3c54)cc(N2c3ccccc3C3Sc4ccccc4C32)c1]

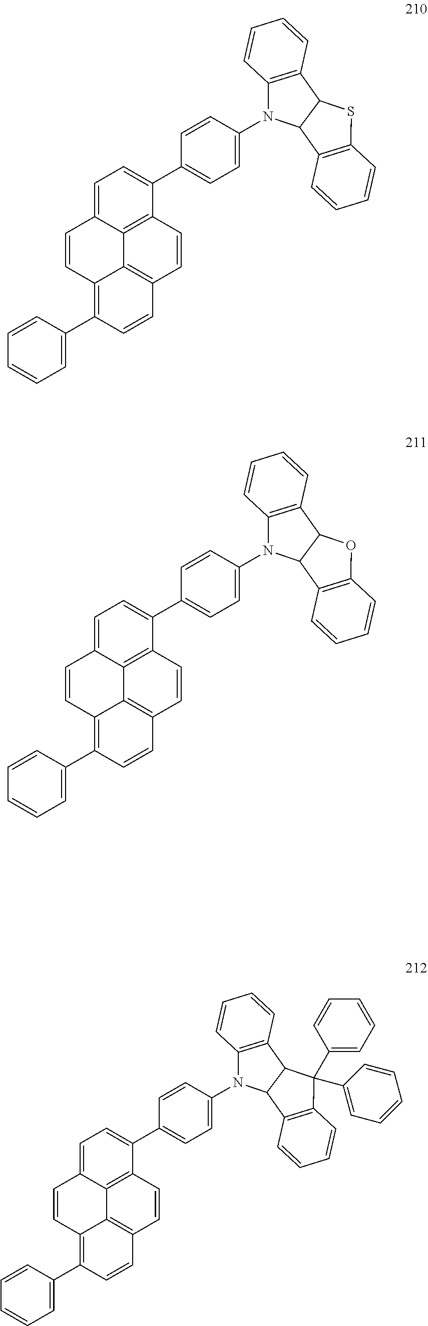 [SMILES: c1ccc(-c2ccc3ccc4c(-c5ccc(N6c7ccccc7C7Sc8ccccc8C76)cc5)ccc5ccc2c3c54)cc1 ; c1ccc(-c2ccc3ccc4c(-c5ccc(N6c7ccccc7C7Oc8ccccc8C76)cc5)ccc5ccc2c3c54)cc1 ; c1ccc(-c2ccc3ccc4c(-c5ccc(N6c7ccccc7C7C6c6ccccc6C7(c6ccccc6)c6ccccc6)cc5)ccc5ccc2c3c54)cc1]

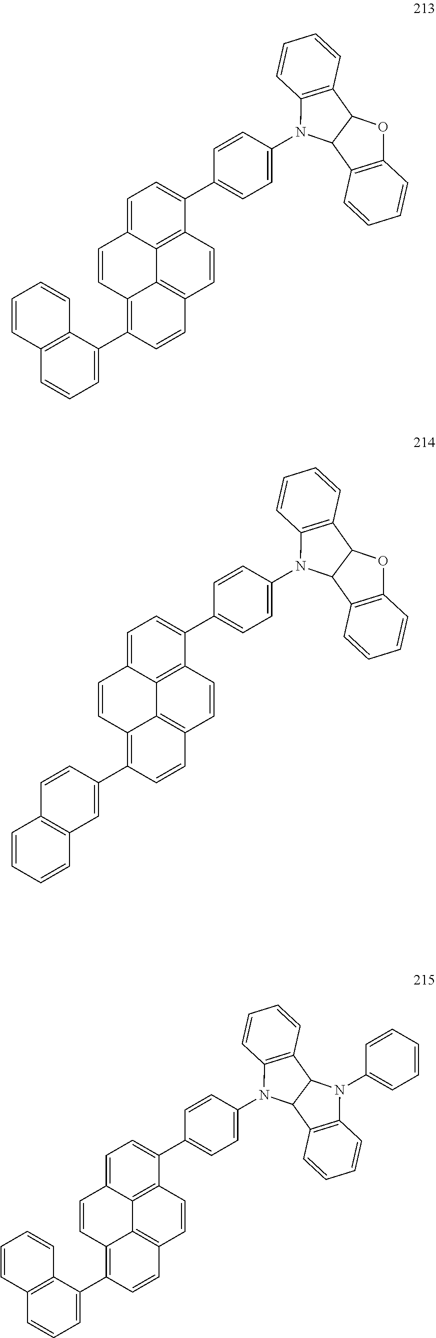 [SMILES: c1ccc2c(c1)OC1c3ccccc3N(c3ccc(-c4ccc5ccc6c(-c7cccc8ccccc78)ccc7ccc4c5c76)cc3)C21 ; c1ccc2c(c1)OC1c3ccccc3N(c3ccc(-c4ccc5ccc6c(-c7ccc8ccccc8c7)ccc7ccc4c5c76)cc3)C21 ; c1ccc(N2c3ccccc3C3C2c2ccccc2N3c2ccc(-c3ccc4ccc5c(-c6cccc7ccccc67)ccc6ccc3c4c65)cc2)cc1]

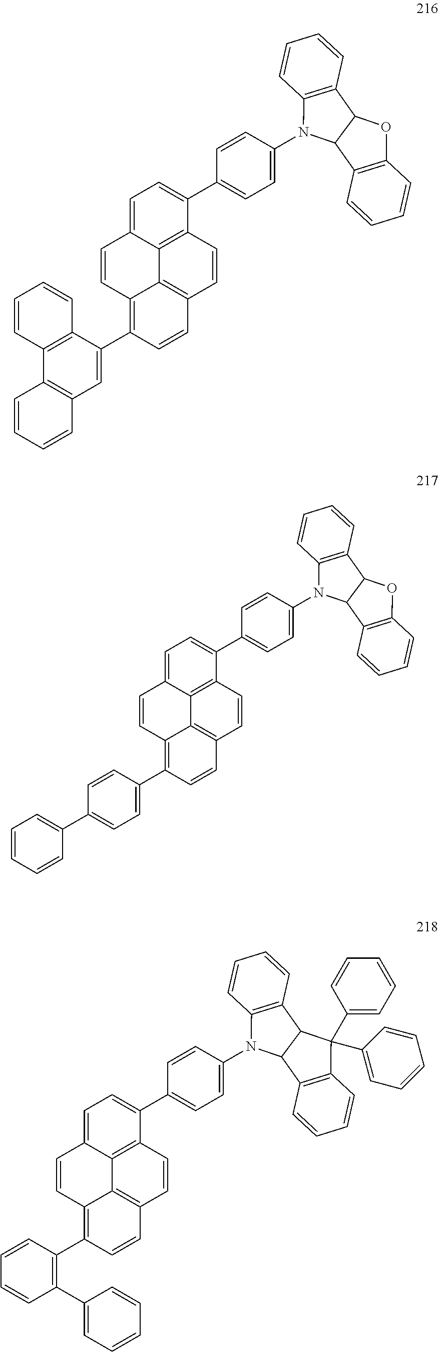 [SMILES: c1ccc2c(c1)OC1c3ccccc3N(c3ccc(-c4ccc5ccc6c(-c7cc8ccccc8c8ccccc78)ccc7ccc4c5c76)cc3)C21 ; c1ccc(-c2ccc(-c3ccc4ccc5c(-c6ccc(N7c8ccccc8C8Oc9ccccc9C87)cc6)ccc6ccc3c4c65)cc2)cc1 ; c1ccc(-c2ccccc2-c2ccc3ccc4c(-c5ccc(N6c7ccccc7C7C6c6ccccc6C7(c6ccccc6)c6ccccc6)cc5)ccc5ccc2c3c54)cc1]

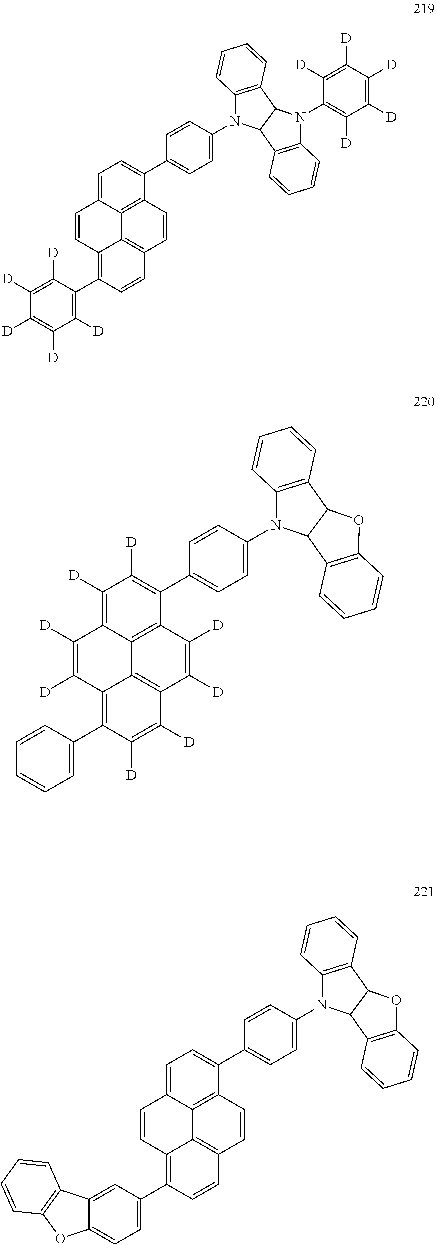 [SMILES: [2H]c1c([2H])c([2H])c(-c2ccc3ccc4c(-c5ccc(N6c7ccccc7C7C6c6ccccc6N7c6c([2H])c([2H])c([2H])c([2H])c6[2H])cc5)ccc5ccc2c3c54)c([2H])c1[2H] ; [2H]c1c([2H])c2c([2H])c([2H])c3c(-c4ccc(N5c6ccccc6C6Oc7ccccc7C65)cc4)c([2H])c([2H])c4c([2H])c([2H])c(c1-c1ccccc1)c2c43 ; c1ccc2c(c1)OC1c3ccccc3N(c3ccc(-c4ccc5ccc6c(-c7ccc8oc9ccccc9c8c7)ccc7ccc4c5c76)cc3)C21]

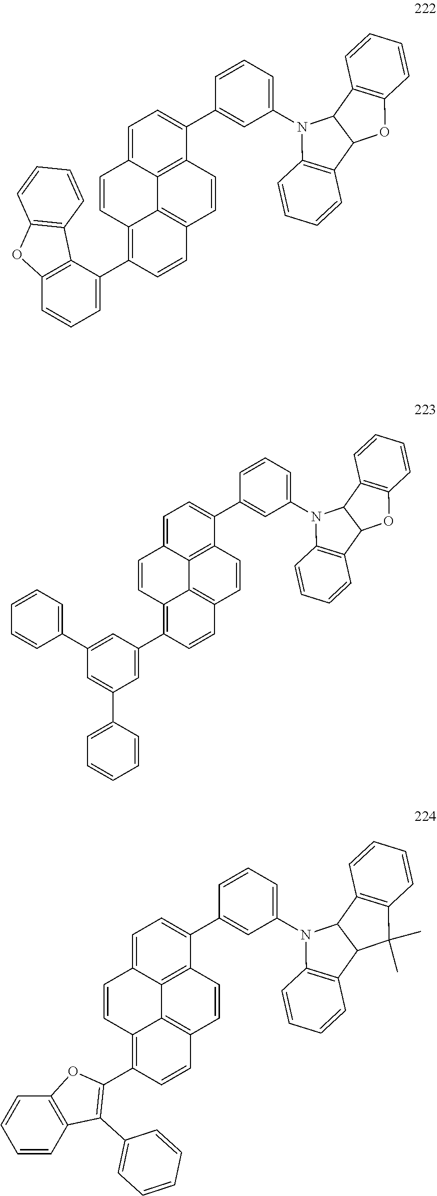 [SMILES: c1cc(-c2ccc3ccc4c(-c5cccc6oc7ccccc7c56)ccc5ccc2c3c54)cc(N2c3ccccc3C3Oc4ccccc4C32)c1 ; c1ccc(-c2cc(-c3ccccc3)cc(-c3ccc4ccc5c(-c6cccc(N7c8ccccc8C8Oc9ccccc9C87)c6)ccc6ccc3c4c65)c2)cc1 ; CC1(C)c2ccccc2C2C1c1ccccc1N2c1cccc(-c2ccc3ccc4c(-c5oc6ccccc6c5-c5ccccc5)ccc5ccc2c3c54)c1]

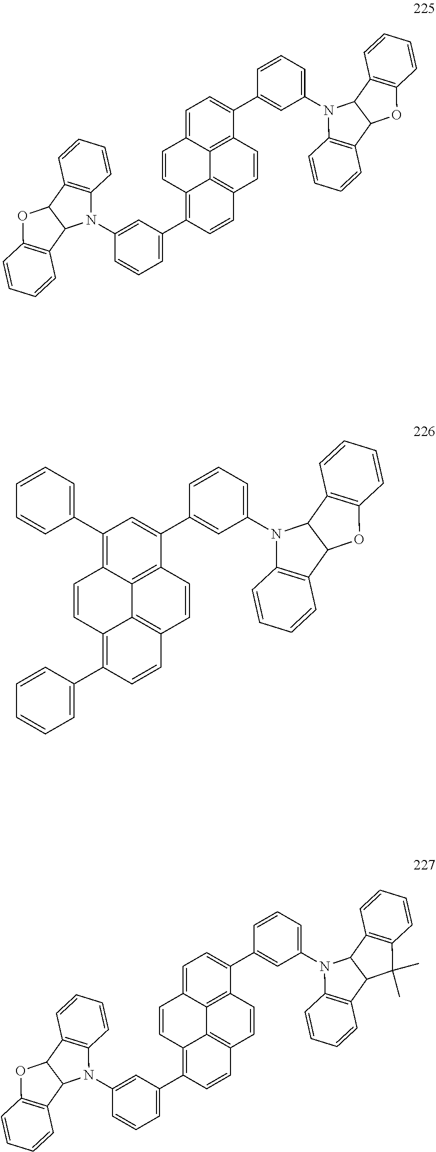 [SMILES: c1cc(-c2ccc3ccc4c(-c5cccc(N6c7ccccc7C7Oc8ccccc8C76)c5)ccc5ccc2c3c54)cc(N2c3ccccc3C3Oc4ccccc4C32)c1 ; c1ccc(-c2ccc3ccc4c(-c5cccc(N6c7ccccc7C7Oc8ccccc8C76)c5)cc(-c5ccccc5)c5ccc2c3c54)cc1 ; CC1(C)c2ccccc2C2C1c1ccccc1N2c1cccc(-c2ccc3ccc4c(-c5cccc(N6c7ccccc7C7Oc8ccccc8C76)c5)ccc5ccc2c3c54)c1]

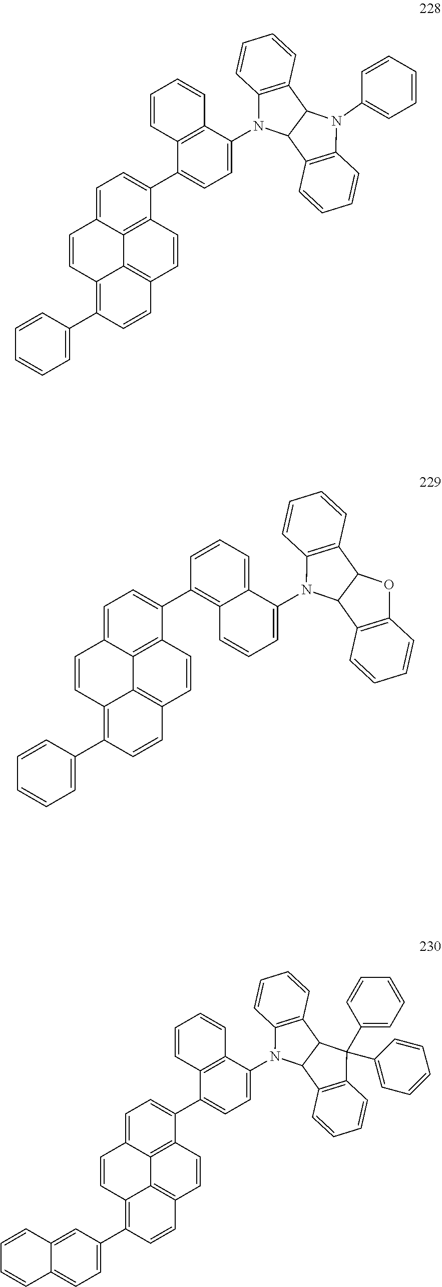 [SMILES: c1ccc(-c2ccc3ccc4c(-c5ccc(N6c7ccccc7C7C6c6ccccc6N7c6ccccc6)c6ccccc56)ccc5ccc2c3c54)cc1 ; c1ccc(-c2ccc3ccc4c(-c5cccc6c(N7c8ccccc8C8Oc9ccccc9C87)cccc56)ccc5ccc2c3c54)cc1 ; c1ccc(C2(c3ccccc3)c3ccccc3C3C2c2ccccc2N3c2ccc(-c3ccc4ccc5c(-c6ccc7ccccc7c6)ccc6ccc3c4c65)c3ccccc23)cc1]

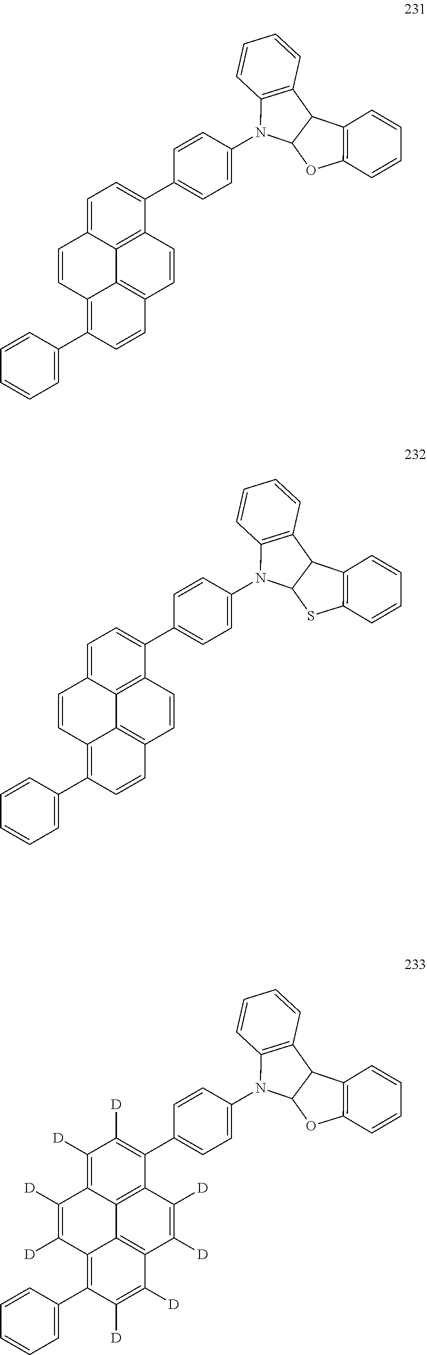 [SMILES: c1ccc(-c2ccc3ccc4c(-c5ccc(N6c7ccccc7C7c8ccccc8OC76)cc5)ccc5ccc2c3c54)cc1 ; c1ccc(-c2ccc3ccc4c(-c5ccc(N6c7ccccc7C7c8ccccc8SC76)cc5)ccc5ccc2c3c54)cc1 ; [2H]c1c([2H])c2c([2H])c([2H])c3c(-c4ccc(N5c6ccccc6C6c7ccccc7OC65)cc4)c([2H])c([2H])c4c([2H])c([2H])c(c1-c1ccccc1)c2c43]

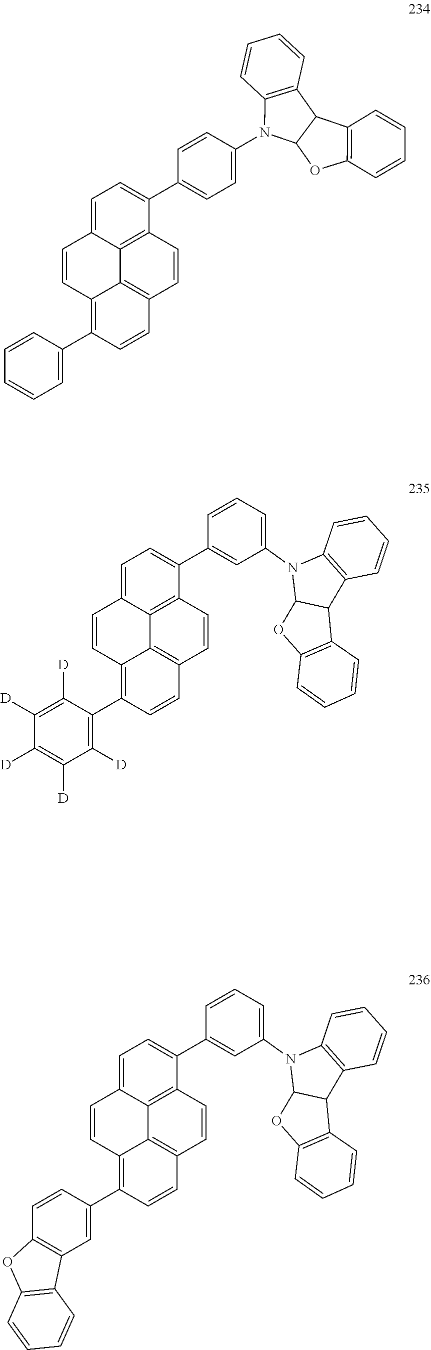 [SMILES: c1ccc(-c2ccc3ccc4c(-c5ccc(N6c7ccccc7C7c8ccccc8OC76)cc5)ccc5ccc2c3c54)cc1 ; [2H]c1c([2H])c([2H])c(-c2ccc3ccc4c(-c5cccc(N6c7ccccc7C7c8ccccc8OC76)c5)ccc5ccc2c3c54)c([2H])c1[2H] ; c1cc(-c2ccc3ccc4c(-c5ccc6oc7ccccc7c6c5)ccc5ccc2c3c54)cc(N2c3ccccc3C3c4ccccc4OC32)c1]

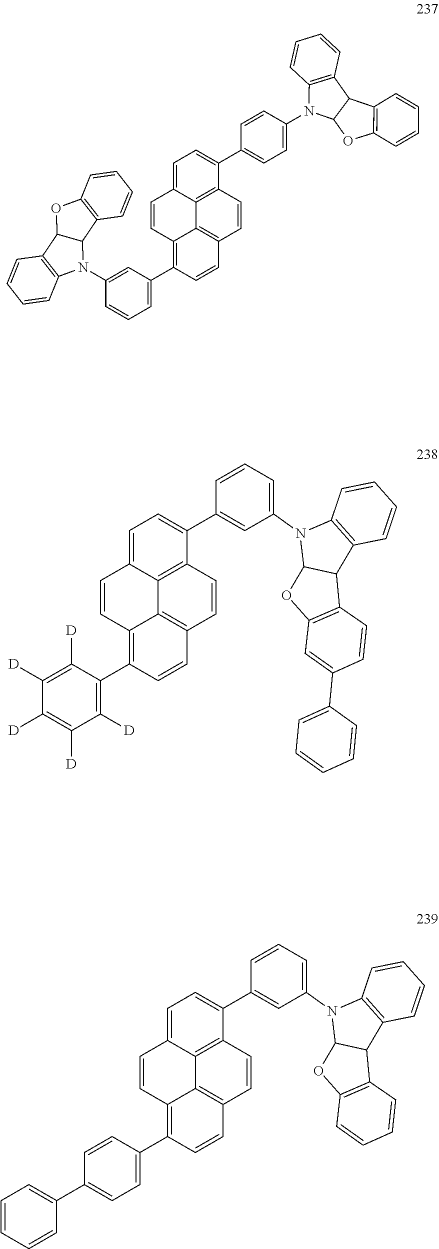 [SMILES: c1cc(-c2ccc3ccc4c(-c5ccc(N6c7ccccc7C7c8ccccc8OC76)cc5)ccc5ccc2c3c54)cc(N2c3ccccc3C3Oc4ccccc4C32)c1 ; [2H]c1c([2H])c([2H])c(-c2ccc3ccc4c(-c5cccc(N6c7ccccc7C7c8ccc(-c9ccccc9)cc8OC76)c5)ccc5ccc2c3c54)c([2H])c1[2H] ; c1ccc(-c2ccc(-c3ccc4ccc5c(-c6cccc(N7c8ccccc8C8c9ccccc9OC87)c6)ccc6ccc3c4c65)cc2)cc1]

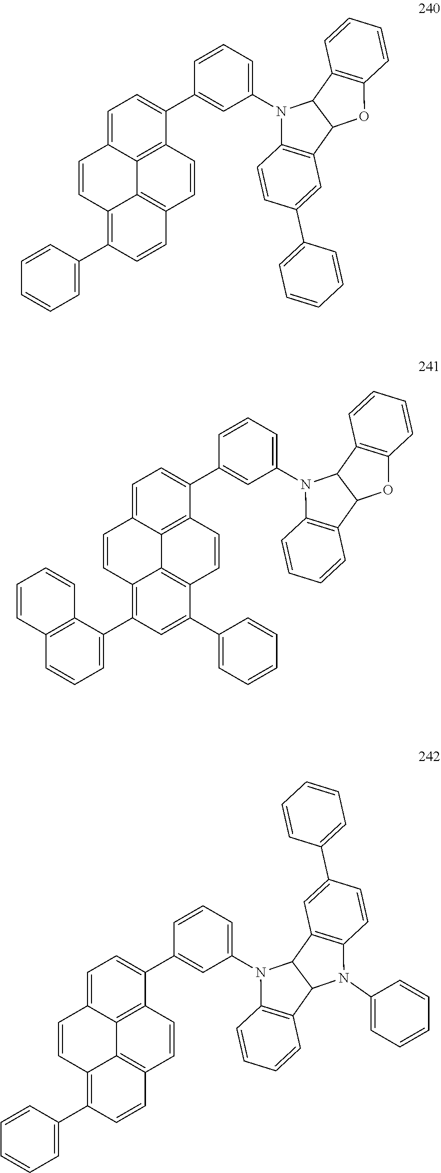 [SMILES: c1ccc(-c2ccc3c(c2)C2Oc4ccccc4C2N3c2cccc(-c3ccc4ccc5c(-c6ccccc6)ccc6ccc3c4c65)c2)cc1 ; c1ccc(-c2cc(-c3cccc4ccccc34)c3ccc4ccc(-c5cccc(N6c7ccccc7C7Oc8ccccc8C76)c5)c5ccc2c3c45)cc1 ; c1ccc(-c2ccc3c(c2)C2C(c4ccccc4N2c2cccc(-c4ccc5ccc6c(-c7ccccc7)ccc7ccc4c5c76)c2)N3c2ccccc2)cc1]

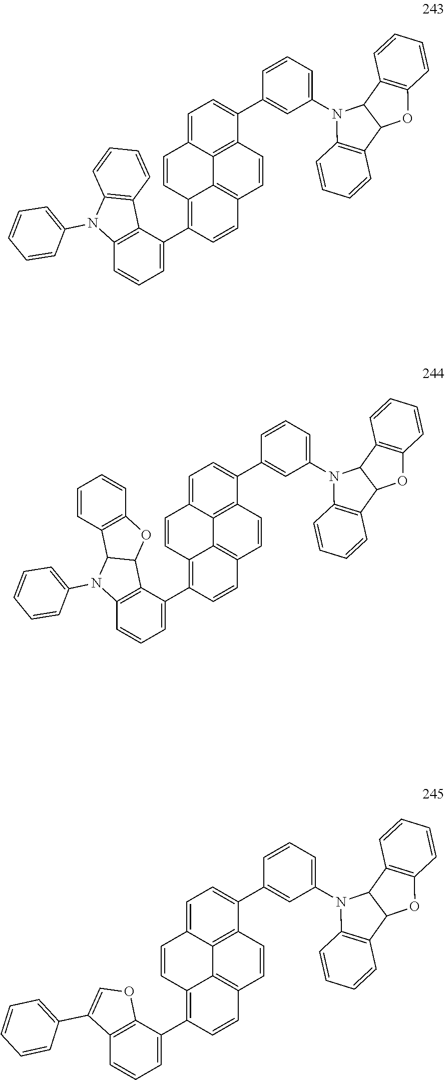 [SMILES: c1ccc(-n2c3ccccc3c3c(-c4ccc5ccc6c(-c7cccc(N8c9ccccc9C9Oc%10ccccc%10C98)c7)ccc7ccc4c5c76)cccc32)cc1 ; c1ccc(N2c3cccc(-c4ccc5ccc6c(-c7cccc(N8c9ccccc9C9Oc%10ccccc%10C98)c7)ccc7ccc4c5c76)c3C3Oc4ccccc4C32)cc1 ; c1ccc(-c2coc3c(-c4ccc5ccc6c(-c7cccc(N8c9ccccc9C9Oc%10ccccc%10C98)c7)ccc7ccc4c5c76)cccc23)cc1]

The organic layers of the organic light emitting device according to the present invention may form a monolayer structure. Alternatively, the organic layers may be stacked together to form a multilayer structure. For example, the organic layers may have a structure including a hole injecting layer, a hole transport layer, a hole blocking layer, a light emitting layer, an electron blocking layer, an electron transport layer, and an electron injecting layer but are not limited to this structure. The number of the organic layers is not limited and may be increased or decreased. Preferred structures of the organic layers of the organic light emitting device according to the present invention will be explained in more detail in the Examples section that follows.

A more detailed description will be given concerning exemplary embodiments of the organic light emitting device according to the present invention.

The organic light emitting device of the present invention includes an anode, a hole transport layer, a light emitting layer, an electron transport layer, and a cathode. The organic light emitting device of the present invention may optionally further include a hole injecting layer between the anode and the hole transport layer and an electron injecting layer between the electron transport layer and the cathode. If necessary, the organic light emitting device of the present invention may further include one or two intermediate layers such as a hole blocking layer or an electron blocking layer. The organic light emitting device of the present invention may further include one or more organic layers such as a capping layer that have various functions depending on the desired characteristics of the device.

A specific structure of the organic light emitting device according to one embodiment of the present invention, a method for fabricating the device, and materials for the organic layers are as follows.

First, an anode material is coated on a substrate to form an anode. The substrate may be any of those used in general organic light emitting devices. The substrate is preferably an organic substrate or a transparent plastic substrate that is excellent in transparency, surface smoothness, ease of handling, and waterproofness. A highly transparent and conductive metal oxide such as indium tin oxide (ITO), indium zinc oxide (IZO), tin oxide (SnO₂) or zinc oxide (ZnO) is used as the anode material.

A hole injecting material is coated on the anode by vacuum thermal evaporation or spin coating to form a hole injecting layer. Then, a hole transport material is coated on the hole injecting layer by vacuum thermal evaporation or spin coating to form a hole transport layer.

The hole injecting material is not specially limited so long as it is usually used in the art. Specific examples of such materials include 4,4′,4″-tris(2-naphthylphenyl-phenylamino)triphenylamine (2-TNATA), N,N′-di(1-naphthyl)-N,N′-diphenylbenzidine (NPD), N,N′-diphenyl-N,N′-bis(3-methylphenyl)-1,1′-biphenyl-4,4′-diamine (TPD), and N,N′-diphenyl-N,N′-bis(4-(phenyl-m-tolylamino)phenyl)biphenyl-4,4′-diamine (DNTPD).

The hole transport material is not specially limited so long as it is commonly used in the art. Examples of such materials include N,N′-bis(3-methylphenyl)-N,N′-diphenyl-(1,1-biphenyl)-4,4′-diamine (TPD) and N,N′-di(naphthalen-1-yl)-N,N′-diphenylbenzidine (α-NPD).

Subsequently, a hole auxiliary layer and a light emitting layer are sequentially laminated on the hole transport layer. A hole blocking layer may be optionally formed on the light emitting layer by vacuum thermal evaporation or spin coating. The hole blocking layer is formed as a thin film and blocks holes from entering a cathode through the organic light emitting layer. This role of the hole blocking layer prevents the lifetime and efficiency of the device from deteriorating. A material having a very low highest occupied molecular orbital (HOMO) energy level is used for the hole blocking layer. The hole blocking material is not particularly limited so long as it can transport electrons and has a higher ionization potential than the light emitting compound. Representative examples of suitable hole blocking materials include BAlq, BCP, and TPBI.

Examples of materials for the hole blocking layer include, but are not limited to, BAlq, BCP, Bphen, TPBI, NTAZ, BeBq₂, OXD-7, and Liq.

An electron transport layer is deposited on the hole blocking layer by vacuum thermal evaporation or spin coating, and an electron injecting layer is formed thereon. A cathode metal is deposited on the electron injecting layer by vacuum thermal evaporation to form a cathode, completing the fabrication of the organic light emitting device.

For example, lithium (Li), magnesium (Mg), aluminum (Al), aluminum-lithium (Al—Li), calcium (Ca), magnesium-indium (Mg—In) or magnesium-silver (Mg—Ag) may be used as the metal for the formation of the cathode. The organic light emitting device may be of top emission type. In this case, a transmissive material such as ITO or IZO may be used to form the cathode.

A material for the electron transport layer functions to stably transport electrons injected from the cathode. The electron transport material may be any of those known in the art and examples thereof include, but are not limited to, quinoline derivatives, particularly tris(8-quinolinolate)aluminum (Alq3), TAZ, Balq, beryllium bis(benzoquinolin-10-olate) (Bebg2), ADN, and oxadiazole derivatives such as PBD, BMD, and BND.

Each of the organic layers can be formed by a monomolecular deposition or solution process. According to the monomolecular deposition process, the material for each layer is evaporated into a thin film under heat and vacuum or reduced pressure. According to the solution process, the material for each layer is mixed with a suitable solvent and the mixture is then formed into a thin film by a suitable method such as ink-jet printing, roll-to-roll coating, screen printing, spray coating, dip coating or spin coating.

The organic light emitting device of the present invention can be used in a display or lighting system selected from flat panel displays, flexible displays, monochromatic flat panel lighting systems, white flat panel lighting systems, flexible monochromatic lighting systems, and flexible white lighting systems.

### MODE FOR CARRYING OUT THE INVENTION

The present invention will be more specifically explained with reference to the following examples. However, it will be obvious to those skilled in the art that these examples are in no way intended to limit the scope of the invention.

### Synthesis Example 1. Synthesis of [46]

### Synthesis Example 1-1. Synthesis of <1-a>

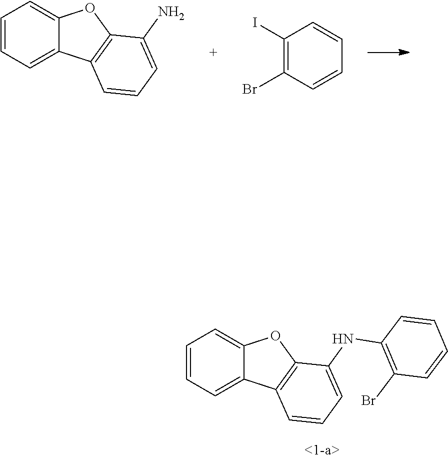 [SMILES: Nc1cccc2c1oc1ccccc12 ; Brc1ccccc1I ; Brc1ccccc1Nc1cccc2c1oc1ccccc12]

To a 500 mL round bottom flask, 4-aminodibenzofuran (25 g, 0.136 mol), 1-bromo-2-iodobenzene (40.5 g, 0.143 mol), palladium acetate (0.6 g, 0.003 mol), 2,2′-bisdiphenylphosphino-1,1′-binaphthyl (1.7 g, 0.003 mol), sodium tert-butoxide (26.2 g, 0.273 mol) and toluene (250 mL) were introduced, and the mixture was refluxed for 6 hours. When the reaction was finished, the reaction solution was cooled to room temperature, and extracted with toluene and water. Then, the organic layer was concentrated, and <1-a> (38 g) was obtained using column chromatography. (Yield 82.4%)

### Synthesis Example 1-2. Synthesis of <1-b>

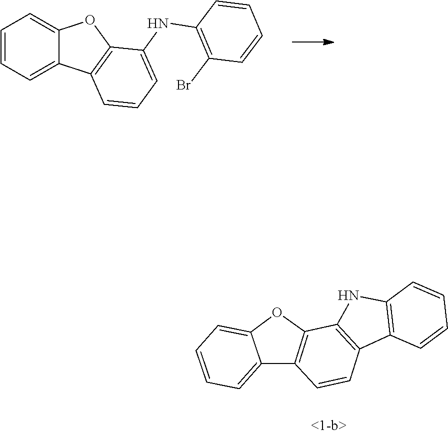 [SMILES: Brc1ccccc1Nc1cccc2c1oc1ccccc12 ; c1ccc2c(c1)[nH]c1c2ccc2c3ccccc3oc21]

To a 500 mL round bottom flask, <1-a> (33.3 g, 0.099 mol), tetrakis(triphenylphosphine)palladium (2.3 g, 0.002 mol), potassium acetate (19.5 g, 0.199 mol) and dimethylformamide (300 mL) were introduced, and the mixture was refluxed for 12 hours. When the reaction was finished, the reaction solution was cooled to room temperature, and then filtered through Celite. After concentrating the filtrate under reduced pressure, <1-b> (17 g) was obtained using column chromatography. (Yield 65.7%)

### Synthesis Example 1-3. Synthesis of <1-c>

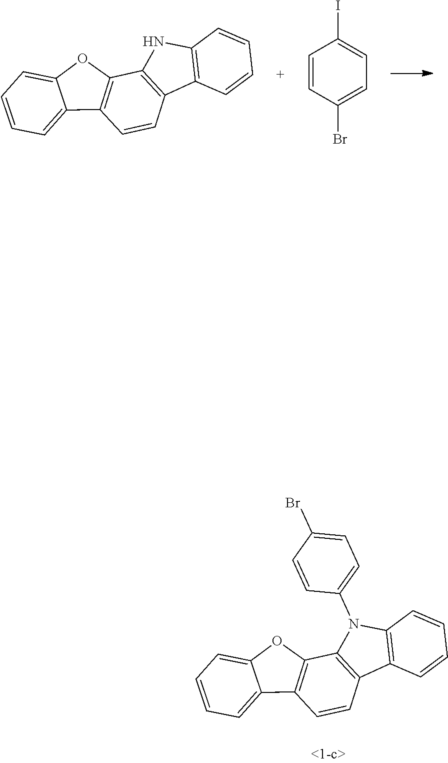 [SMILES: c1ccc2c(c1)[nH]c1c2ccc2c3ccccc3oc21 ; Brc1ccc(I)cc1 ; Brc1ccc(-n2c3ccccc3c3ccc4c5ccccc5oc4c32)cc1]

To a 500 mL round bottom flask <1-b> (12.6 g, 0.049 mol), 1-bromo-4-iodobenzene (20.6 g, 0.073 mol), copper iodide (0.5 g, 0.002 mol), potassium phosphate (31 g, 0.146 mol), 1,2-cyclohexanediamine (cis:trans=1:1) (16.7 g, 0.146 mol) and 1,4-dioxane (140 mL) were introduced, and the mixture was refluxed for 6 hours. When the reaction was finished, the reaction solution was cooled to room temperature, and then filtered through Celite. After concentrating the filtrate under reduced pressure, <1-c> (14.5 g) was obtained using column chromatography. (Yield 72.1%)

### Synthesis Example 1-4. Synthesis of <1-d>

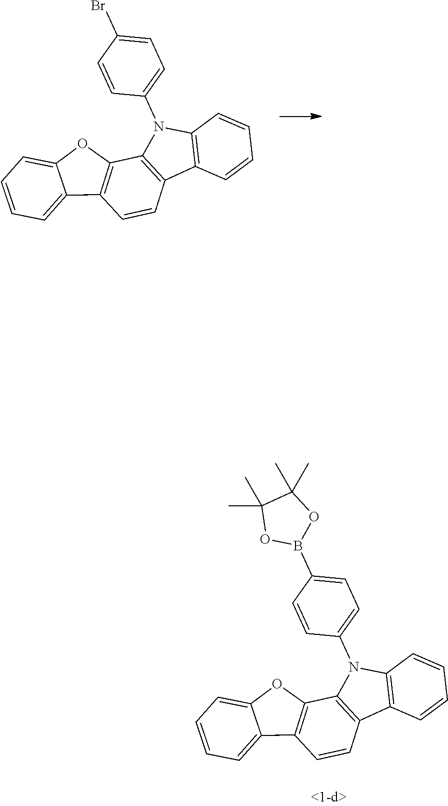 [SMILES: Brc1ccc(-n2c3ccccc3c3ccc4c5ccccc5oc4c32)cc1 ; CC1(C)OB(c2ccc(-n3c4ccccc4c4ccc5c6ccccc6oc5c43)cc2)OC1(C)C]

To a 300 mL round bottom flask under a nitrogen atmosphere, <1-c> (13.2 g, 0.032 mol), bis(pinacolato)diboron (10.5 g, 0.041 mol), bis(diphenylphosphino)ferrocene dichloropalladium (0.5 g, 0.001 mol), potassium acetate (7.8 g, 0.079 mol) and 1,4-dioxane (150 mL) were introduced, and the mixture was refluxed for 7 hours. When the reaction was finished, the reaction solution was cooled to room temperature, and then filtered through Celite. After concentrating the filtrate, <1-d> (9 g) was obtained using column chromatography. (Yield 61.2%)

### Synthesis Example 1-5. Synthesis of <1-e>

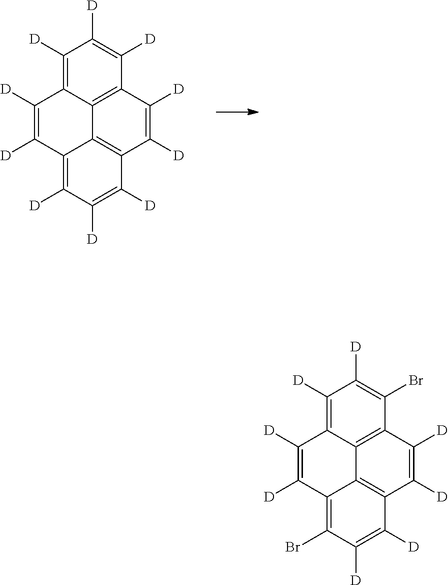 [SMILES: [2H]c1c([2H])c2c([2H])c([2H])c3c([2H])c([2H])c([2H])c4c([2H])c([2H])c(c1[2H])c2c34 ; [2H]c1c([2H])c2c([2H])c([2H])c3c(Br)c([2H])c([2H])c4c([2H])c([2H])c(c1Br)c2c43]

To a 2000 mL reactor under a nitrogen atmosphere, pyrene (D10) (40 g, 0.188 mol) and dichloromethane (800 mL) were introduced, and the mixture was stirred. After lowering the temperature inside the reactor to 0° C. or lower, a solution in which bromine (58.7 g, 0.367 mol) and dichloromethane (200 mL) were mixed was slowly added dropwise thereto. After the dropwise addition was completed, the temperature was raised to room temperature, and the result was stirred for 3 hours. After the reaction was finished, an aqueous sodium thiosulfate solution was introduced to the reaction solution, and the result was stirred for 1 hour and then filtered. The solid was purified with 1,2-dichlorobenzene to obtain <1-e> (31 g). (Yield 44.7%)

### Synthesis Example 1-6. Synthesis of <1-f>

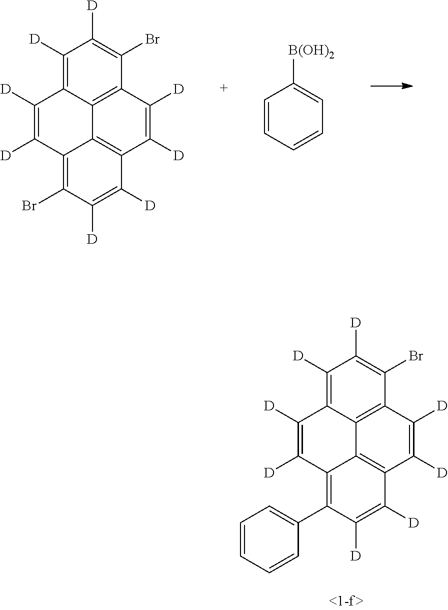 [SMILES: [2H]c1c([2H])c2c([2H])c([2H])c3c(Br)c([2H])c([2H])c4c([2H])c([2H])c(c1Br)c2c43 ; OB(O)c1ccccc1 ; [2H]c1c([2H])c2c([2H])c([2H])c3c(-c4ccccc4)c([2H])c([2H])c4c([2H])c([2H])c(c1Br)c2c43]

To a 3000 mL round bottom flask purged with nitrogen, <1-e> (102 g, 0.278 mol), phenylboronic acid (35.3 g, 0.278 mol), tetrakis(triphenylphosphine)palladium (Pd[PPh₃]₄) (6.4 g, 0.006 mol), sodium carbonate (88.3 g, 0.833 mol), toluene (1400 mL) and water (420 mL) was introduced, and the mixture was refluxed for 9 hours. When the reaction was finished, the result was cooled to room temperature, and then the produced solid was filtered and discarded. The filtrate was extracted with ethyl acetate and water, and then the organic layer was subjected to anhydrous treatment. After the anhydrous treatment, the organic layer was concentrated under reduced pressure, and <1-f> (69.4 g) was obtained through column chromatography. (Yield 68.4%)

### Synthesis Example 1-7. Synthesis of [46]

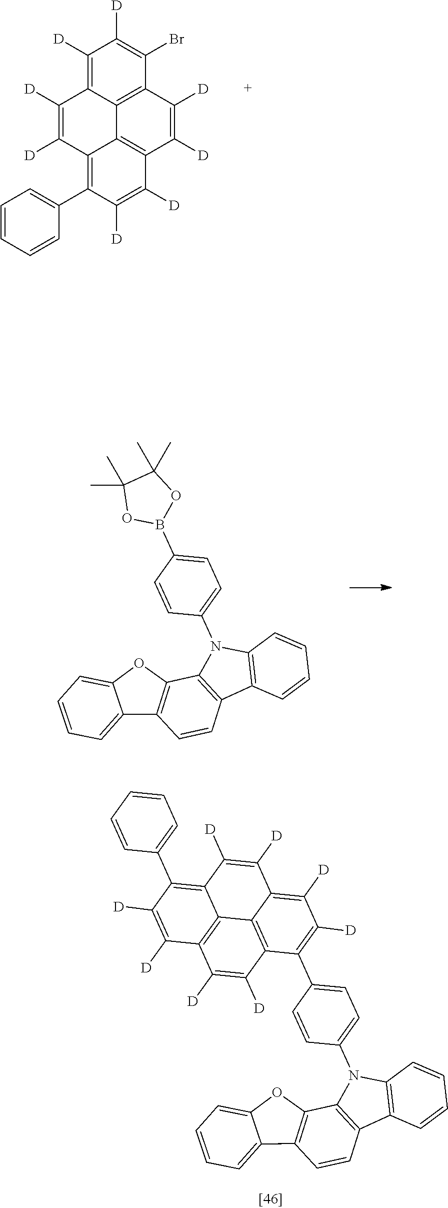 [SMILES: [2H]c1c([2H])c2c([2H])c([2H])c3c(-c4ccccc4)c([2H])c([2H])c4c([2H])c([2H])c(c1Br)c2c43 ; CC1(C)OB(c2ccc(-n3c4ccccc4c4ccc5c6ccccc6oc5c43)cc2)OC1(C)C ; [2H]c1c([2H])c2c([2H])c([2H])c3c(-c4ccc(-n5c6ccccc6c6ccc7c8ccccc8oc7c65)cc4)c([2H])c([2H])c4c([2H])c([2H])c(c1-c1ccccc1)c2c43]

To a 300 mL round bottom flask under a nitrogen atmosphere, <1-f> (8 g, 0.022 mol), <1-d> (11.5 g, 0.025 mol), tetrakis(triphenylphosphine)palladium (0.5 g, 0.001 mol), potassium carbonate (5.3 g, 0.038 mol), toluene (56 mL), ethanol (14 mL) and water (19 mL) were introduced, and the mixture was refluxed for 6 hours. After the reaction was finished, the result was cooled to room temperature, and extracted using ethyl acetate and water. The organic layer was subjected to anhydrous treatment and then concentrated, and [46] (7.6 g) was obtained using column chromatography. (Yield 56%)

MS(MALDI-TOF): m/z 617.26[M]⁺

### Synthesis Example 2. Synthesis of [48]

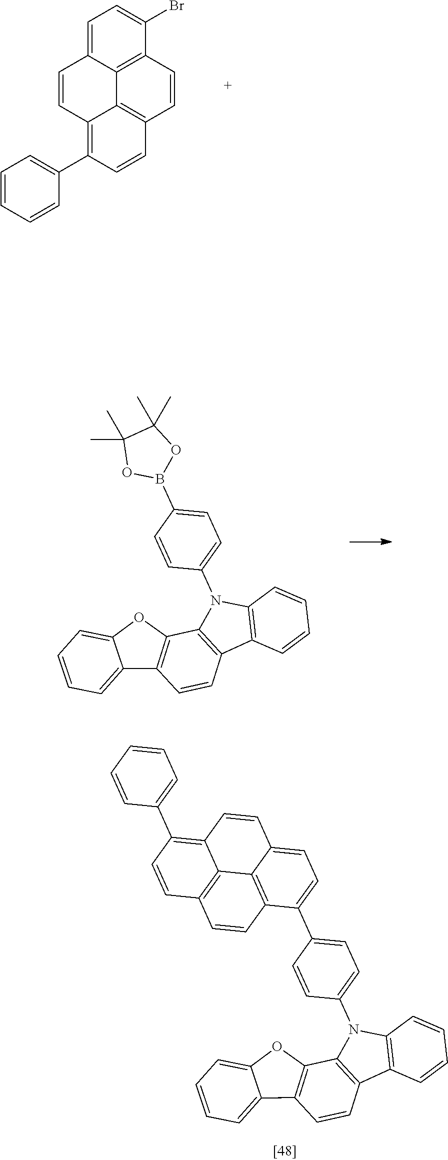 [SMILES: Brc1ccc2ccc3c(-c4ccccc4)ccc4ccc1c2c43 ; CC1(C)OB(c2ccc(-n3c4ccccc4c4ccc5c6ccccc6oc5c43)cc2)OC1(C)C ; c1ccc(-c2ccc3ccc4c(-c5ccc(-n6c7ccccc7c7ccc8c9ccccc9oc8c76)cc5)ccc5ccc2c3c54)cc1]

[48] was obtained in the same manner as in Synthesis Example 1-7, except that 1-bromo-6-phenylpyrene was used instead of <1-f>. (Yield 67%)

MS(MALDI-TOF): m/z 609.21[M]⁺

### Synthesis Example 3. Synthesis of [62]

### Synthesis Example 3-1. Synthesis of <3-a>

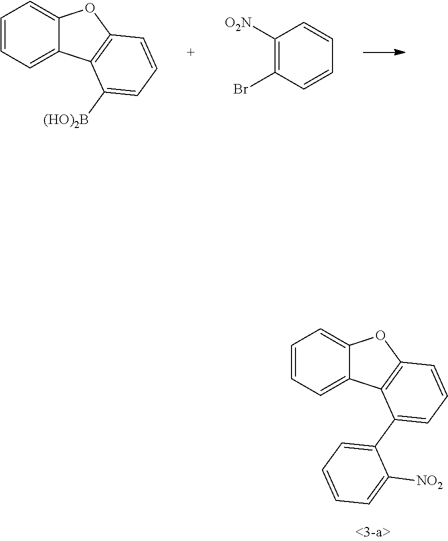 [SMILES: OB(O)c1cccc2oc3ccccc3c12 ; O=[N+]([O-])c1ccccc1Br ; O=[N+]([O-])c1ccccc1-c1cccc2oc3ccccc3c12]

To a 500 mL round bottom flask under a nitrogen atmosphere, 2-bromonitrobenzene (23.5 g, 0.116 mol), dibenzofuran-1-ylboronic acid (28.1 g, 0.133 mol), tetrakis(triphenylphosphine)palladium (2.7 g, 0.002 mol), potassium carbonate (27.3 g, 0.198 mol), toluene (165 mL), ethanol (40 mL) and water (100 mL) were introduced, and the mixture was refluxed for 8 hours. When the reaction was finished, the reaction solution was cooled to room temperature, then extracted with ethyl acetate and water, and the organic layer was concentrated under reduced pressure. After the concentration under reduced pressure, <3-a> (27.6 g) was obtained using column chromatography. (Yield 82.4%)

### Synthesis Example 3-2. Synthesis of <3-b>

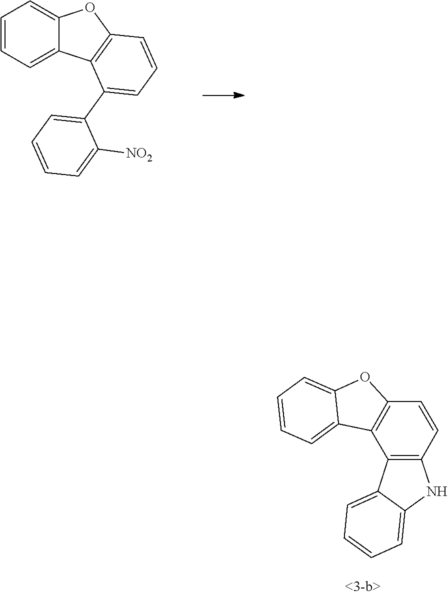 [SMILES: O=[N+]([O-])c1ccccc1-c1cccc2oc3ccccc3c12 ; c1ccc2c(c1)[nH]c1ccc3oc4ccccc4c3c12]

To a 500 mL round bottom flask, <3-a> (22.2 g, 0.077 mol), triphenylphosphine (62.2 g, 0.231 mol) and 1,2-dichlorobenzene (246 mL) were introduced, and the mixture was refluxed for 18 hours. When the reaction was finished, the reaction solution was cooled to room temperature, and then extracted with dichloromethane and water, and then the organic layer was concentrated under reduced pressure. After the concentration under reduced pressure, <3-b> (14.3 g) was obtained using column chromatography. (Yield 72.3%)

### Synthesis Example 3-3. Synthesis of <3-c>

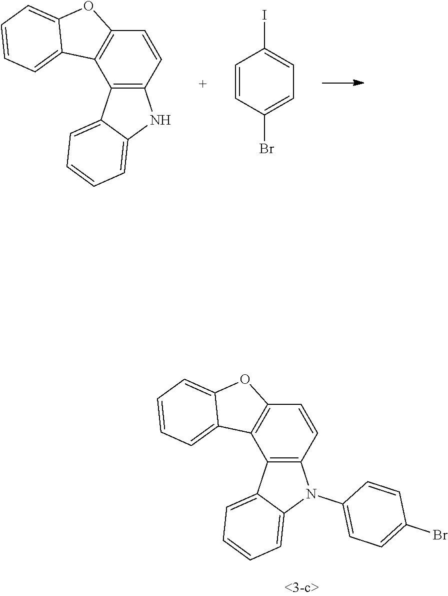 [SMILES: c1ccc2c(c1)[nH]c1ccc3oc4ccccc4c3c12 ; Brc1ccc(I)cc1 ; Brc1ccc(-n2c3ccccc3c3c4c(ccc32)oc2ccccc24)cc1]

<3-c> was obtained in the same manner as in Synthesis Example 1-3, except that <3-b> was used instead of <1-b>. (Yield 73.9%)

### Synthesis Example 3-4. Synthesis of <3-d>

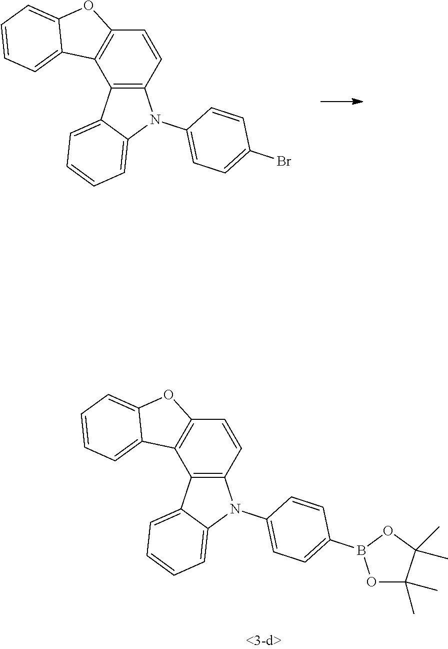 [SMILES: Brc1ccc(-n2c3ccccc3c3c4c(ccc32)oc2ccccc24)cc1 ; CC1(C)OB(c2ccc(-n3c4ccccc4c4c5c(ccc43)oc3ccccc35)cc2)OC1(C)C]

<3-d> was obtained in the same manner as in Synthesis Example 1-4, except that <3-c> was used instead of <1-c>. (Yield 51.9%)

### Synthesis Example 3-5. Synthesis of [62]

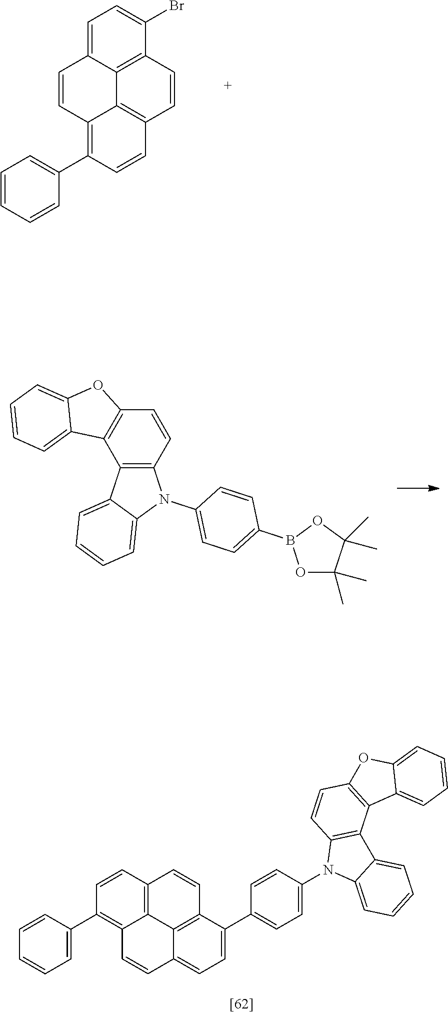 [SMILES: Brc1ccc2ccc3c(-c4ccccc4)ccc4ccc1c2c43 ; CC1(C)OB(c2ccc(-n3c4ccccc4c4c5c(ccc43)oc3ccccc35)cc2)OC1(C)C ; c1ccc(-c2ccc3ccc4c(-c5ccc(-n6c7ccccc7c7c8c(ccc76)oc6ccccc68)cc5)ccc5ccc2c3c54)cc1]

[62] was obtained in the same manner as in Synthesis Example 1-7, except that 1-bromo-6-phenylpyrene was used instead of <1-f> and <3-d> was used instead of <1-d>. (Yield 67%) MS(MALDI-TOF): m/z 617.26[M]⁺

### Synthesis Example 4. Synthesis of [63]

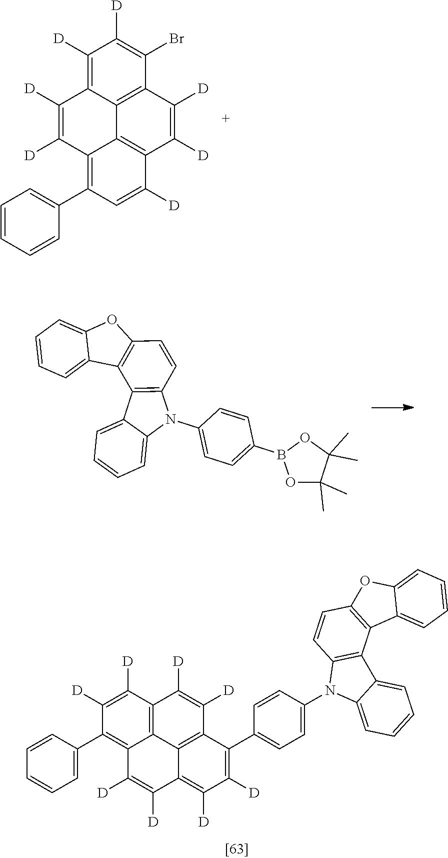 [SMILES: [2H]c1c([2H])c2c([2H])c([2H])c3c(-c4ccccc4)cc([2H])c4c([2H])c([2H])c(c1Br)c2c43 ; CC1(C)OB(c2ccc(-n3c4ccccc4c4c5c(ccc43)oc3ccccc35)cc2)OC1(C)C ; [2H]c1c([2H])c2c([2H])c([2H])c3c(-c4ccc(-n5c6ccccc6c6c7c(ccc65)oc5ccccc57)cc4)c([2H])c([2H])c4c([2H])c([2H])c(c1-c1ccccc1)c2c43]

[63] was obtained in the same manner as in Synthesis Example 3-5, except that <1-f> was used instead of 1-bromo-6-phenylpyrene. (Yield 73%)

MS(MALDI-TOF): m/z 617.26[M]⁺

### Synthesis Example 5. Synthesis of [201]

### Synthesis Example 5-1. Synthesis of <5-a>

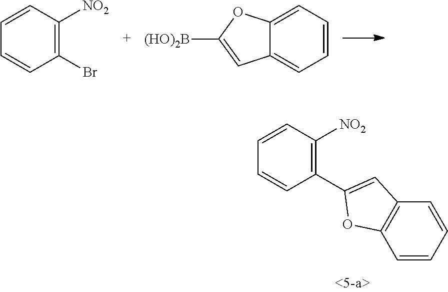 [SMILES: O=[N+]([O-])c1ccccc1Br ; OB(O)c1cc2ccccc2o1 ; O=[N+]([O-])c1ccccc1-c1cc2ccccc2o1]

To a round bottom flask, 2-bromonitrobenzene (25 g, 0.124 mol), benzofuran-2-ylboronic acid (24.1 g, 0.149 mol), tetrakis(triphenylphosphine)palladium (4.3 g, 0.004 mol), potassium carbonate (51.3 g, 0.371 mol), toluene (175 mL), ethanol (44 mL) and water (185 mL) were introduced, and the mixture was refluxed for 12 hours. When the reaction was finished, the reaction solution was cooled to room temperature, then extracted with ethyl acetate and water, and concentrated under reduced pressure. After the concentration, the result was separated by column chromatography to obtain <5-a> (21.6 g). (Yield 73%)

### Synthesis Example 5-2. Synthesis of <5-b>

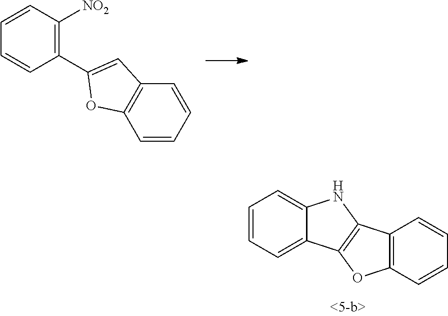 [SMILES: O=[N+]([O-])c1ccccc1-c1cc2ccccc2o1 ; c1ccc2c(c1)[nH]c1c3ccccc3oc21]

To a round bottom flask, <5-a> (21.6 g, 0.090 mol), triphenylphosphine (72.9 g, 0.271 mol) and 1,2-dichlorobenzene (216 mL) were introduced, and the mixture was refluxed for 24 hours. When the reaction was finished, the reaction solution was cooled to room temperature, then poured into water (300 mL), and the result was stirred for 1 hour, and then extracted with dichloromethane and water. The organic layer was concentrated under reduced pressure, and then separated by column chromatography to obtain <5-b> (15 g). (Yield 80%)

### Synthesis Example 5-3. Synthesis of <5-c>

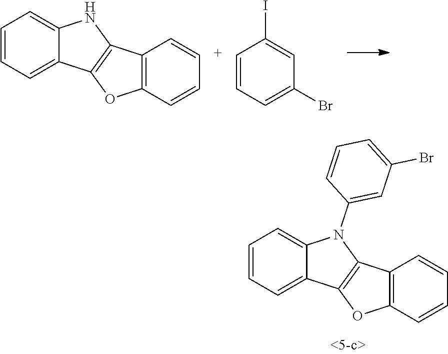

To a round bottom flask, <5-b> (30 g, 0.145 mol), 1-bromo-3-iodobenzene (61.4 g, 0.217 mol), copper iodide (1.4 g, 0.007 mol), potassium phosphate (61.5 g, 0.290 mol), 1,2-cyclohexanediamine (cis:trans 50:50) (49.6 g, 0.288 mol) and 1,4-dioxane (240 mL) were introduced, and the mixture was refluxed for 12 hours. When the reaction was finished, the reaction solution was cooled to room temperature, then filtered through Celite, and the filtrate was concentrated under reduced pressure. After the concentration, the result was crystallized using methanol and then filtered, and the filtered solid was recrystallized with dichloromethane and methanol to obtain <5-c> (39 g). (Yield 74.4%)

### Synthesis Example 5-4. Synthesis of <5-d>

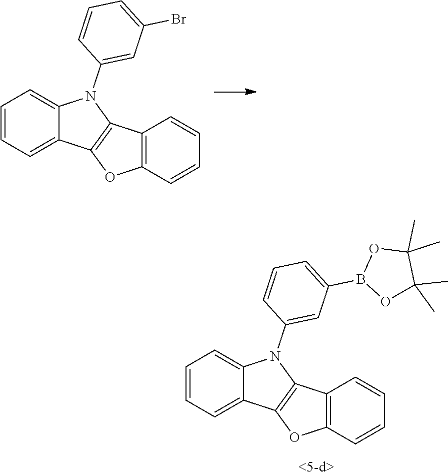 [SMILES: Brc1cccc(-n2c3ccccc3c3oc4ccccc4c32)c1 ; CC1(C)OB(c2cccc(-n3c4ccccc4c4oc5ccccc5c43)c2)OC1(C)C]

To a round bottom flask, <5-c> (39 g, 0.108 mol), bis(pinacolato)diboron (32.8 g, 0.129 mol), bis(diphenylphosphino)ferrocene dichloropalladium (1.8 g, 0.002 mol), calcium acetate (26.4 g, 0.269 mol) and 1,4-dioxane (390 mL) were introduced, and the mixture was refluxed for 12 hours. When the reaction was finished, the reaction solution was cooled to room temperature, and then filtered through Celite. The filtrate was concentrated, and then separated by column chromatography to obtain <5-d> (26 g). (Yield 59%)

### Synthesis Example 5-5. Synthesis of [201]

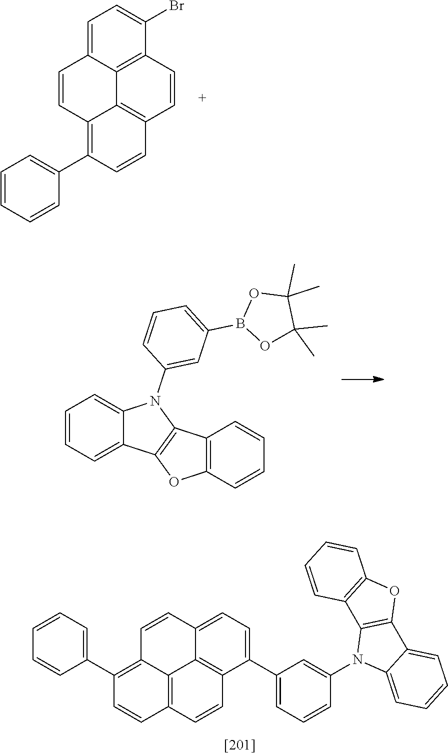 [SMILES: Brc1ccc2ccc3c(-c4ccccc4)ccc4ccc1c2c43 ; CC1(C)OB(c2cccc(-n3c4ccccc4c4oc5ccccc5c43)c2)OC1(C)C ; c1ccc(-c2ccc3ccc4c(-c5cccc(-n6c7ccccc7c7oc8ccccc8c76)c5)ccc5ccc2c3c54)cc1]

To a round bottom flask under a nitrogen atmosphere, 1-bromo-6-phenylpyrene (10 g, 0.028 mol), <5-d> (12.6 g, 0.031 mol), tetrakis(triphenylphosphine)palladium (0.6 g, 0.001 mol), potassium carbonate (6.6 g, 0.048 mol), toluene (70 mL), ethanol (17 mL) and water (24 mL) were introduced, and the mixture was refluxed for 6 hours. When the reaction was finished, the reaction solution was cooled to room temperature, and then extracted with ethyl acetate and water. The organic layer was concentrated under reduced pressure, then crystallized with methanol, and then filtered. The filtered solid was recrystallized to obtain [201] (7.9 g). (Yield 50.4%)

MS(MALDI-TOF): m/z 559.19 [M]⁺

### Synthesis Example 6. Synthesis of [203]

### Synthesis Example 6-1. Synthesis of <6-a>

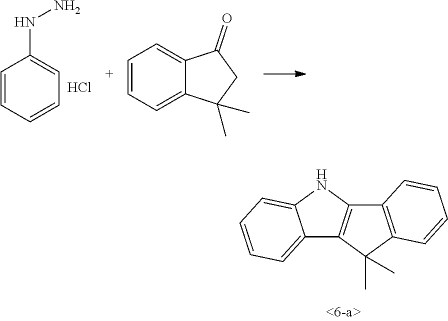 [SMILES: NNc1ccccc1 ; Cl ; CC1(C)CC(=O)c2ccccc21 ; CC1(C)c2ccccc2-c2[nH]c3ccccc3c21]

To a round bottom flask, phenylhydrazine hydrochloride (100 g, 0.692 mol), 3,3-dimethyl-1-indanone (166.2 g, 1.037 mol), acetic acid (800 mL) and hydrochloric acid (30 mL) were introduced, and the mixture was refluxed for 24 hours. When the reaction was finished, the reaction solution was cooled to room temperature, then poured into water (1000 mL), and the result was stirred for 1 hour, and then extracted with ethyl acetate and water. The organic layer was concentrated under reduced pressure, and then separated by column chromatography to obtain <6-a> (76 g). (Yield 47.1%)

### Synthesis Example 6-2. Synthesis of <6-b>

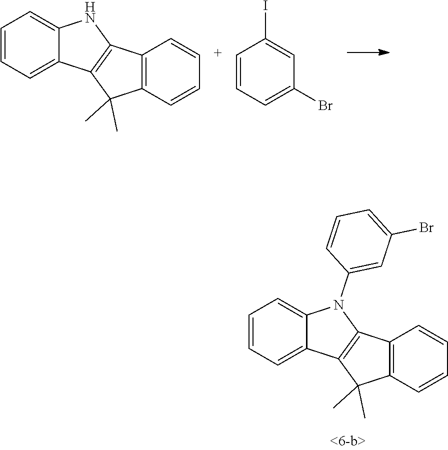

<6-b> (22 g) was obtained in the same manner as in Synthesis Example 5-3, except that <6-a> was used instead of <5-b>. (Yield 68%)

### Synthesis Example 6-3. Synthesis of <6-c>

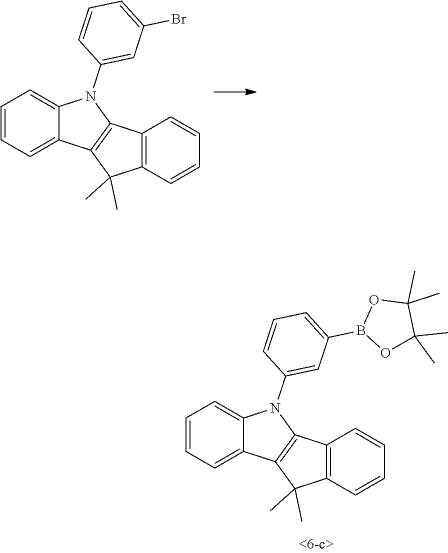 [SMILES: CC1(C)c2ccccc2-c2c1c1ccccc1n2-c1cccc(Br)c1 ; CC1(C)c2ccccc2-c2c1c1ccccc1n2-c1cccc(B2OC(C)(C)C(C)(C)O2)c1]

<6-c> (13 g) was obtained in the same manner as in Synthesis Example 5-4, except that <6-b> was used instead of <5-c>. (Yield 53%)

### Synthesis Example 6-4. Synthesis of [203]

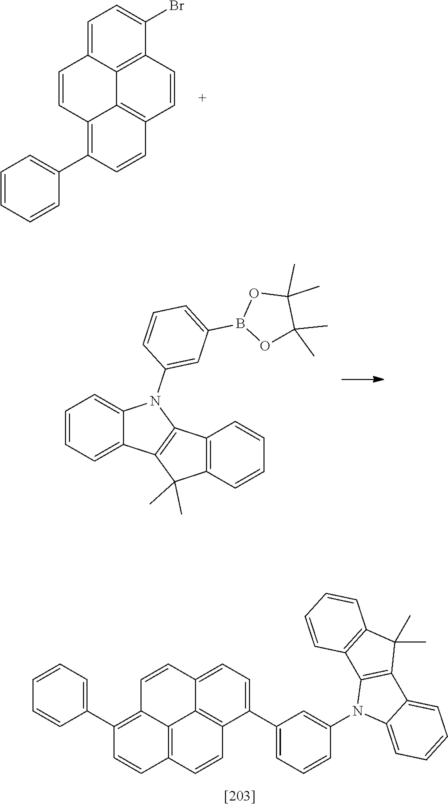 [SMILES: Brc1ccc2ccc3c(-c4ccccc4)ccc4ccc1c2c43 ; CC1(C)c2ccccc2-c2c1c1ccccc1n2-c1cccc(B2OC(C)(C)C(C)(C)O2)c1 ; CC1(C)c2ccccc2-c2c1c1ccccc1n2-c1cccc(-c2ccc3ccc4c(-c5ccccc5)ccc5ccc2c3c54)c1]

[203] (8.3 g) was obtained in the same manner as in Synthesis Example 5-5, except that <6-c> was used instead of <5-d>. (Yield 76%)

MS(MALDI-TOF): m/z 585.25 [M]⁺

### Synthesis Example 7. Synthesis of [234]

### Synthesis Example 7-1. Synthesis of <7-a>

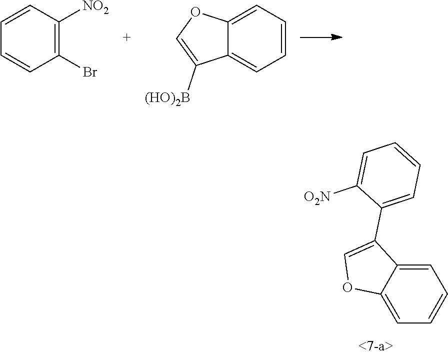 [SMILES: O=[N+]([O-])c1ccccc1Br ; OB(O)c1coc2ccccc12 ; O=[N+]([O-])c1ccccc1-c1coc2ccccc12]

<7-a> was obtained in the same manner as in Synthesis Example 5-1, except that B-3-benzofuranylboronic acid was used instead of benzofuran-2-ylboronic acid. (Yield 70%)

### Synthesis Example 7-2. Synthesis of <7-b>

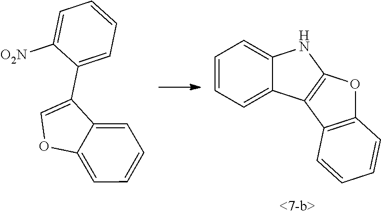 [SMILES: O=[N+]([O-])c1ccccc1-c1coc2ccccc12 ; c1ccc2c(c1)[nH]c1oc3ccccc3c12]

<7-b> was obtained in the same manner as in Synthesis Example 5-2, except that <7-a> was used instead of <5-a>. (Yield 82%)

### Synthesis Example 7-3. Synthesis of <7-c>

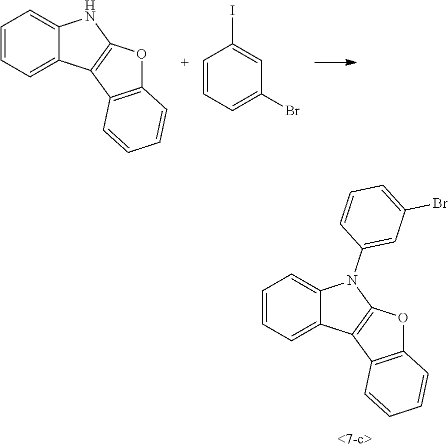

<7-c> was obtained in the same manner as in Synthesis Example 5-3, except that <7-b> was used instead of <5-b>. (Yield 78%)

### Synthesis Example 7-4. Synthesis of <7-d>

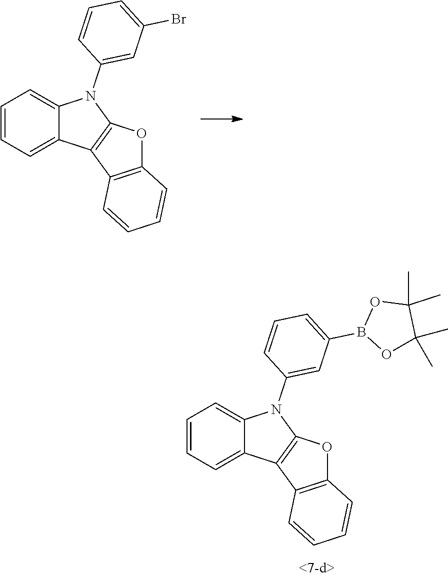 [SMILES: Brc1cccc(-n2c3ccccc3c3c4ccccc4oc32)c1 ; CC1(C)OB(c2cccc(-n3c4ccccc4c4c5ccccc5oc43)c2)OC1(C)C]

<7-d> was obtained in the same manner as in Synthesis Example 5-4, except that <7-c> was used instead of <5-c>. (Yield 58%)

### Synthesis Example 7-5. Synthesis of [234]

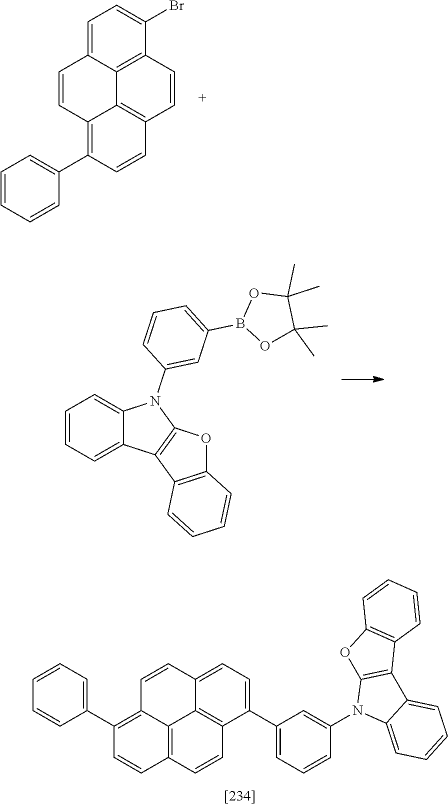 [SMILES: Brc1ccc2ccc3c(-c4ccccc4)ccc4ccc1c2c43 ; CC1(C)OB(c2cccc(-n3c4ccccc4c4c5ccccc5oc43)c2)OC1(C)C ; c1ccc(-c2ccc3ccc4c(-c5cccc(-n6c7ccccc7c7c8ccccc8oc76)c5)ccc5ccc2c3c54)cc1]

[234] was obtained in the same manner as in Synthesis Example 5-5, except that <7-d> was used instead of <5-d>. (Yield 54%)

MS(MALDI-TOF): m/z 559.19 [M]⁺

### Examples 1 to 7: Manufacture of Organic Light Emitting Device

An ITO glass was patterned to have a light emitting area of 2 mm×2 mm, and then cleaned. The ITO glass was installed in a vacuum chamber, and after setting the base pressure at 1×10⁻⁷ torr, 2-TNA TA (400 Å) and HT (200 Å) were deposited on the ITO in this order. A host compound according to the present invention and a dopant compound (BD) described below were mixed in 3 wt % and deposited (250 Å) as a light emitting layer. Then, [Formula E-1] (300 Å) was deposited as an electron transport layer and Liq (10 Å) was deposited as an electron injecting layer sequentially, and Al (1000 Å), a cathode, was deposited to manufacture an organic light emitting device. Luminous characteristics of the organic light emitting device were measured at 10 mA/cm².

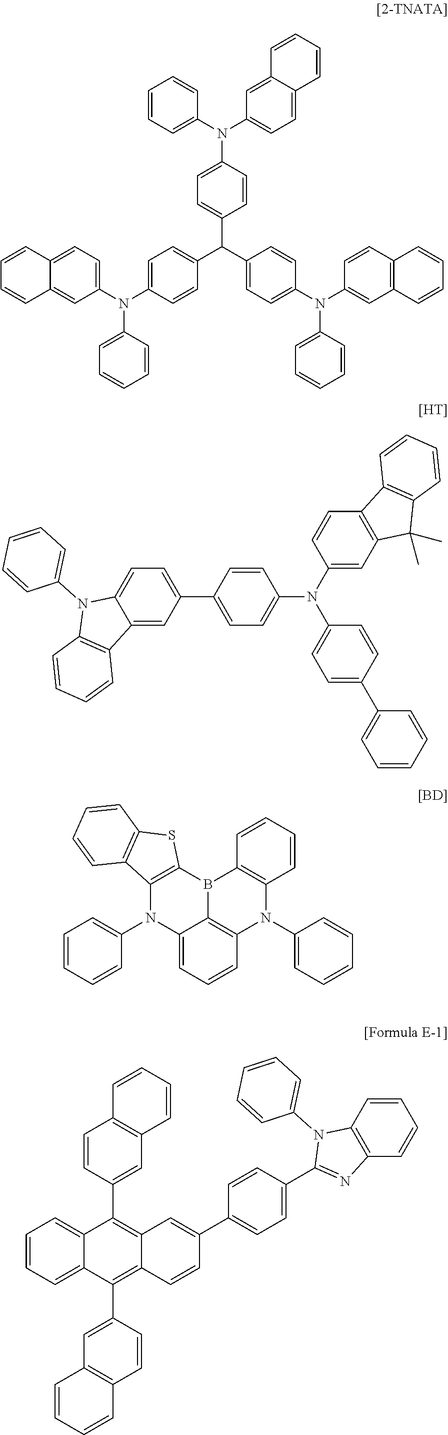 [SMILES: c1ccc(N(c2ccc(C(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N2c3ccccc3B3c4sc5ccccc5c4N(c4ccccc4)c4cccc2c43)cc1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1]

### Comparative Examples 1 to 4

Organic light emitting devices for Comparative Examples were manufactured in the same manner as in Examples, except that the following [BH1] to [BH4] were used as the host compound instead of the compound according to the present invention in the device structure of Examples, and luminous characteristics of the organic light emitting device were measured at 10 mA/cm². The structures of [BH1] to [BH4] are as follows.

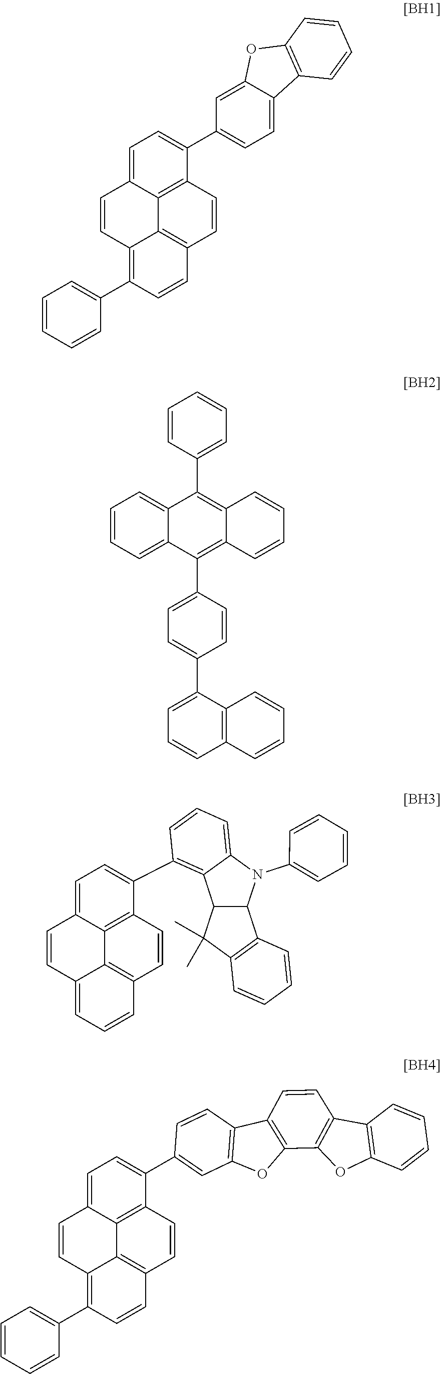

TABLE 1

| Category | Host | V | EQE | T97 |
|---|---|---|---|---|
| Example 1 | 46 | 3.8 | 12.1 | 250 |
| Example 2 | 48 | 3.7 | 11.8 | 225 |
| Example 3 | 62 | 3.7 | 11.7 | 235 |
| Example 4 | 63 | 3.6 | 12.0 | 250 |
| Example 5 | 201 | 3.8 | 11.2 | 220 |
| Example 6 | 203 | 3.7 | 11.4 | 215 |
| Example 7 | 234 | 3.6 | 11.2 | 220 |
| Comparative Example 1 | BH1 | 4.2 | 9.3 | 150 |
| Comparative Example 2 | BH2 | 4.0 | 9.7 | 135 |
| Comparative Example 3 | BH3 | 3.9 | 10.8 | 200 |
| Comparative Example 4 | BH4 | 3.8 | 10.6 | 185 |

As shown in [Table 1], the device employing the compound according to the present invention as a light emitting layer host compound in the organic light emitting device is capable of lower voltage driving and has significantly superior quantum efficiency and lifetime properties compared to each of the devices employing a compound having a difference compared to the characteristic structures of the compound according to the present invention and an anthracene derivative widely used in the related art (Comparative Examples 1 to 4), thereby accomplishing a high-efficiency and long-lifetime organic light emitting device.

### INDUSTRIAL APPLICABILITY

An organic light emitting device according to the present invention may be embodied as a high-efficiency, long-lifetime organic light emitting device with excellent luminous characteristics in emission efficiency and the like by employing a polycyclic compound having a characteristic structure with a pyrene derivative incorporated thereinto as a host in a light emitting layer, and therefore, is industrially useful in illumination devices as well as various display devices such as flat panel, flexible and wearable displays.

1. A compound represented by Formula 1 or Formula 2: 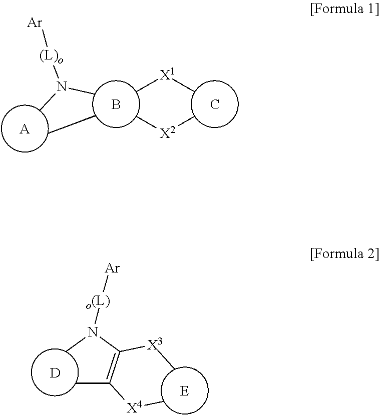
wherein, in [Formula 1] and [Formula 2],
rings A to E are each independently substituted or unsubstituted C₆-C₂₀ aryl;
X¹ to X⁴ are each independently a single bond, or CRR′, O, S or NR (however, cases where both X¹ and X² in [Formula 1] are a single bond and both X³ and X⁴ in [Formula 2] are a single bond are excluded);
R and R′ are each independently any one selected from hydrogen, deuterium, halogen, cyano, substituted or unsubstituted C₁-C₃₀ alkyl, substituted or unsubstituted C₃-C₃₀ cycloalkyl, substituted or unsubstituted C₁-C₃₀ heterocycloalkyl, substituted or unsubstituted C₆-C₅₀ aryl, substituted or unsubstituted C₂-C₅₀ heteroaryl, substituted or unsubstituted C₀-C₃₀ amine, substituted or unsubstituted C₀-C₃₀ silyl, substituted or unsubstituted C₁-C₃₀ alkoxy and substituted or unsubstituted C₆-C₅₀ aryloxy;
R and R′ in CRR′ are optionally linked to each other to form an alicyclic, aromatic monocyclic or polycyclic ring;
Ls are each independently a single bond, or substituted or unsubstituted C₁-C₁₀ alkylene, substituted or unsubstituted C₆-C₂₀ arylene, or substituted or unsubstituted C₅-C₂₀ heteroarylene;
o is from 1 to 3, and when o is 2 or greater, a plurality of Ls are each the same as or different from each other; and
Ar is represented by the following [Structural Formula A], 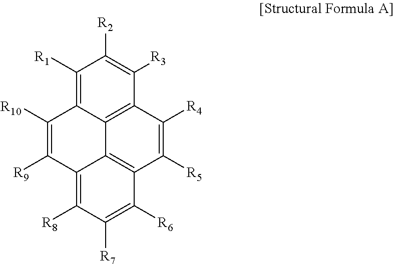
in [Structural Formula A],
any one of R₁ to R₁₀ is linked to L of [Formula 1] or [Formula 2]; and
the rest of R₁ to R₁₀ not linked to L are the same as or different from each other, and each independently any one selected from hydrogen, deuterium, halogen, cyano, substituted or unsubstituted C₁-C₃₀ alkyl, substituted or unsubstituted C₃-C₃₀ cycloalkyl, substituted or unsubstituted C₁-C₃₀ heterocycloalkyl, substituted or unsubstituted C₆-C₅₀ aryl, substituted or unsubstituted C₂-C₅₀ heteroaryl, substituted or unsubstituted C₀-C₃₀ amine, substituted or unsubstituted C₀-C₃₀ silyl, substituted or unsubstituted C₁-C₃₀ alkoxy and substituted or unsubstituted C₆-C₅₀ aryloxy.

2. The compound according to claim 1, wherein the term “substituted” in the definition of the substituents in Formula 1, Formula 2 and Structural Formula indicates substitution with one or more substituents selected from the group consisting of deuterium, cyano, halogen, hydroxyl, nitro, C₁-C₂₄ alkyl, C₃-C₂₄ cycloalkyl, C₁-C₂₄ haloalkyl, C₂-C₂₄ alkenyl, C₂-C₂₄ alkynyl, C₃-C₂₄ cycloalkyl, C₁-C₂₄ heteroalkyl, C₆-C₂₄ aryl, C₇-C₂₄ arylalkyl, C₇-C₂₄ alkylaryl, C₂-C₂₄ heteroaryl, C₂-C₂₄ heteroarylalkyl, C₁-C₂₄ alkoxy, C₁-C₂₄ alkylamino, C₁₂-C₂₄ diarylamino, C₂-C₂₄ diheteroarylamino, C₇-C₂₄ aryl(heteroaryl)amino, C₁-C₂₄ alkylsilyl, C₆-C₂₄ arylsilyl, C₆-C₂₄ aryloxy, and C₆-C₂₄ arylthionyl, more preferably deuterium, cyano, halogen, hydroxyl, nitro, C₁-C₁₂ alkyl, C₁-C₁₂ haloalkyl, C₂-C₁₂ alkenyl, C₂-C₁₂ alkynyl, C₃-C₁₂ cycloalkyl, C₁-C₁₂ heteroalkyl, C₆-C₁₈ aryl, C₇-C₂₀ arylalkyl, C₇-C₂₀ alkylaryl, C₂-C₁₈ heteroaryl, C₂-C₁₈ heteroarylalkyl, C₁-C₁₂ alkoxy, C₁-C₁₂ alkylamino, C₁₂-C₁₈ diarylamino, C₂-C₁₈ diheteroarylamino, C₇-C₁₈ aryl(heteroaryl)amino, C₁-C₁₂ alkylsilyl, C₆-C₁₈ arylsilyl, C₆-C₁₈ aryloxy, and C₆-C₁₈ arylthionyl, or a combination thereof. The term “unsubstituted” in the same definition indicates having no substituent. One or more hydrogen atoms in each of the substituents are optionally replaced by deuterium atoms.

3. The compound according to claim 1, wherein L is substituted or unsubstituted C₆-C₂₀ arylene.

4. The compound according to claim 3, wherein L is any one selected from the following [Structural Formula 1] to [Structural Formula 5]: 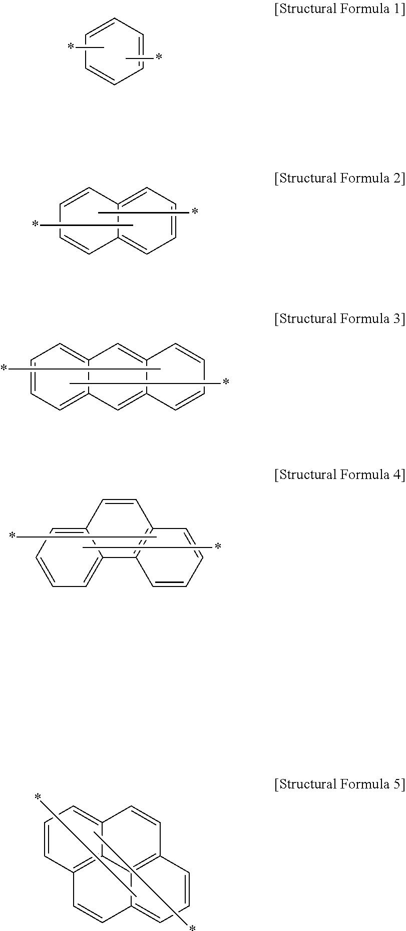
hydrogen atoms at the carbon sites of the aromatic rings represented by [Structural Formula 1] to [Structural Formula 5] are optionally substituted with deuterium atoms.

5. The compound according to claim 1, wherein the compound represented by [Formula 1] is represented by any one selected from the following [Formula 1-1] to [Formula 1-6]: 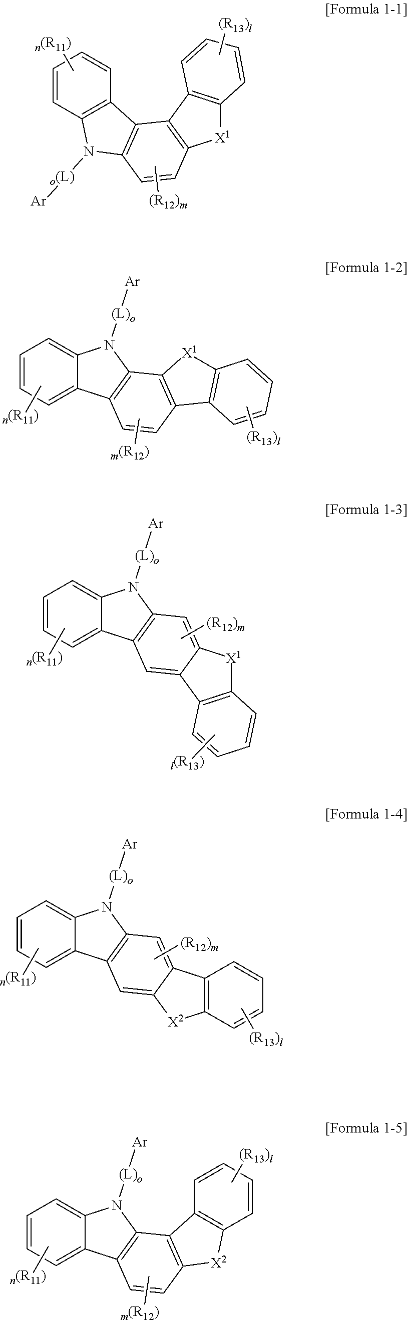 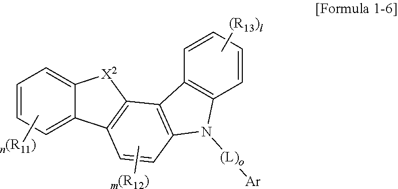
in [Formula 1-1] to [Formula 1-6],
X₁ and X₂, L, o and Ar each have the same definitions as in [Formula 1];
R₁₁ to R₁₃ are the same as or different from each other, and each independently any one selected from hydrogen, deuterium, halogen, cyano, substituted or unsubstituted C₁-C₃₀ alkyl, substituted or unsubstituted C₃-C₃₀ cycloalkyl, substituted or unsubstituted C₁-C₃₀ heterocycloalkyl, substituted or unsubstituted C₆-C₅₀ aryl, substituted or unsubstituted C₂-C₅₀ heteroaryl, substituted or unsubstituted C₀-C₃₀ amine, substituted or unsubstituted C₀-C₃₀ silyl, substituted or unsubstituted C₁-C₃₀ alkoxy and substituted or unsubstituted C₆-C₅₀ aryloxy; and
l and n are each an integer of 1 to 4, m is an integer of 1 to 2, and when 1, n and m are each 2 or greater, a plurality of R₁₁s to R₁₃s are each the same as or different from each other.

6. The compound according to claim 1, wherein the compound represented by [Formula 2] is represented by any one selected from the following [Formula 2-1] and [Formula 2-2]: 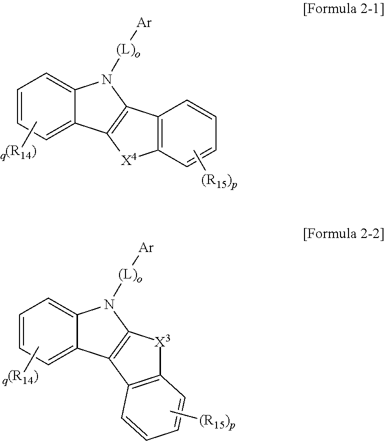
in [Formula 2-1] and [Formula 2-2],
X₃ and X₄, L, o and Ar have the same definitions as in [Formula 2];
R₁₄ and R₁₅ are the same as or different from each other, and each independently any one selected from hydrogen, deuterium, halogen, cyano, substituted or unsubstituted C₁-C₃₀ alkyl, substituted or unsubstituted C₃-C₃₀ cycloalkyl, substituted or unsubstituted C₁-C₃₀ heterocycloalkyl, substituted or unsubstituted C₆-C₅₀ aryl, substituted or unsubstituted C₂-C₅₀ heteroaryl, substituted or unsubstituted C₀-C₃₀ amine, substituted or unsubstituted C₀-C₃₀ silyl, substituted or unsubstituted C₁-C₃₀ alkoxy and substituted or unsubstituted C₆-C₅₀ aryloxy; and
p and q are each an integer of 1 to 4, and when p and q are each 2 or greater, a plurality of R₁₄s and R₁₅s are each the same as or different from each other.

7. The compound according to claim 1, wherein any one selected from R₁, R₃, R₆ and R₈ in [Structural Formula A] is linked to the linker L of [Formula 1] or [Formula 2].

8. The compound according to claim 1, wherein the compound represented by Formula 1 is selected from the following compounds 1 to 114:

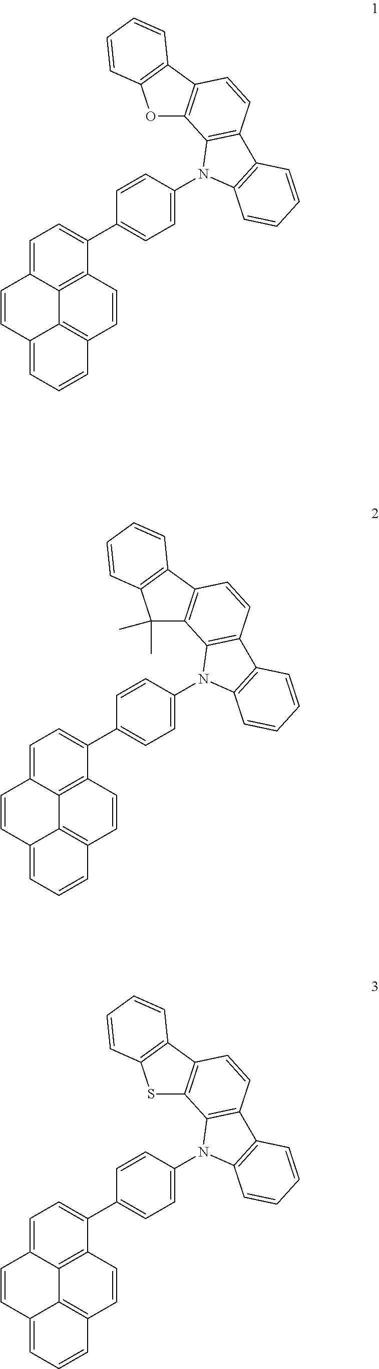 [SMILES: c1cc2ccc3ccc(-c4ccc(-n5c6ccccc6c6ccc7c8ccccc8oc7c65)cc4)c4ccc(c1)c2c34 ; CC1(C)c2ccccc2-c2ccc3c4ccccc4n(-c4ccc(-c5ccc6ccc7cccc8ccc5c6c78)cc4)c3c21 ; c1cc2ccc3ccc(-c4ccc(-n5c6ccccc6c6ccc7c8ccccc8sc7c65)cc4)c4ccc(c1)c2c34]

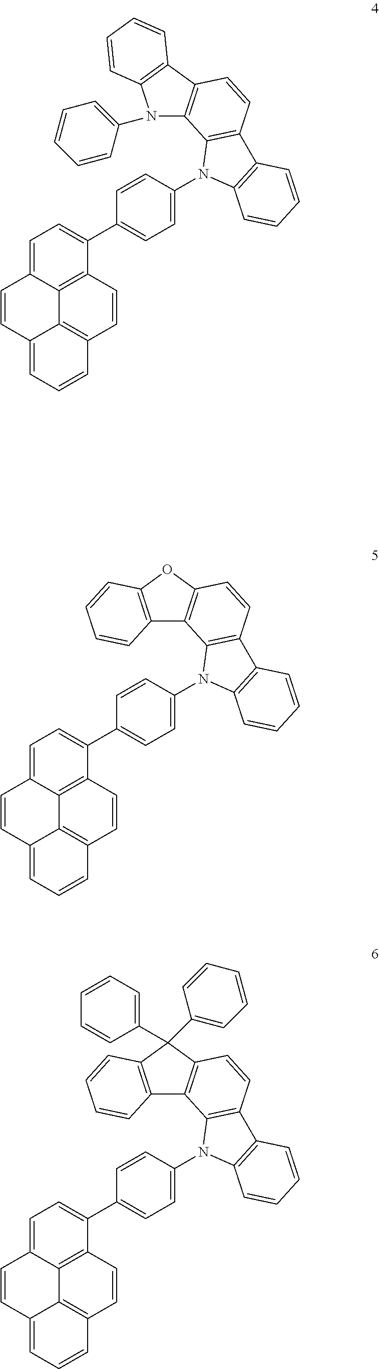 [SMILES: c1ccc(-n2c3ccccc3c3ccc4c5ccccc5n(-c5ccc(-c6ccc7ccc8cccc9ccc6c7c89)cc5)c4c32)cc1 ; c1cc2ccc3ccc(-c4ccc(-n5c6ccccc6c6ccc7oc8ccccc8c7c65)cc4)c4ccc(c1)c2c34 ; c1ccc(C2(c3ccccc3)c3ccccc3-c3c2ccc2c4ccccc4n(-c4ccc(-c5ccc6ccc7cccc8ccc5c6c78)cc4)c32)cc1]

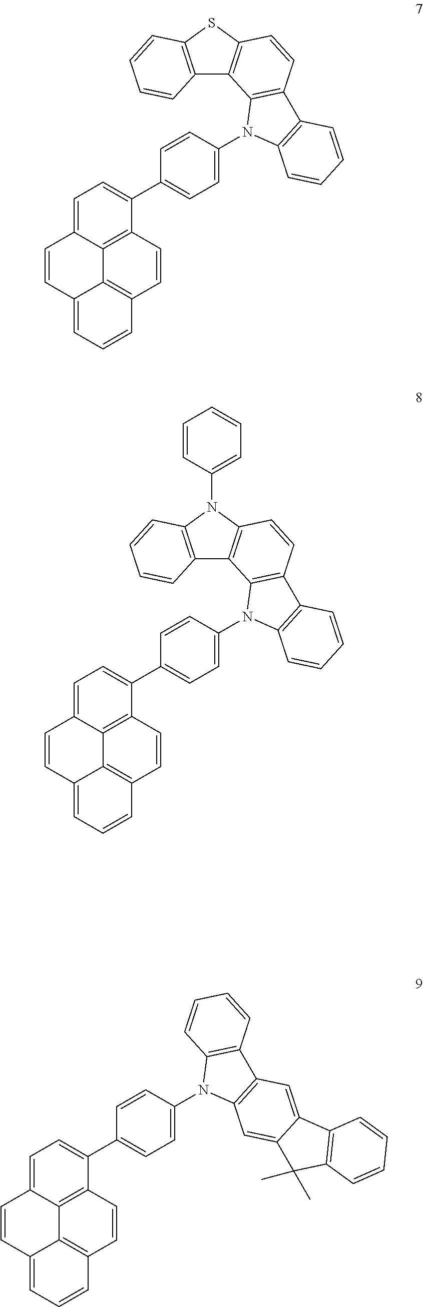 [SMILES: c1cc2ccc3ccc(-c4ccc(-n5c6ccccc6c6ccc7sc8ccccc8c7c65)cc4)c4ccc(c1)c2c34 ; c1ccc(-n2c3ccccc3c3c2ccc2c4ccccc4n(-c4ccc(-c5ccc6ccc7cccc8ccc5c6c78)cc4)c23)cc1 ; CC1(C)c2ccccc2-c2cc3c4ccccc4n(-c4ccc(-c5ccc6ccc7cccc8ccc5c6c78)cc4)c3cc21]

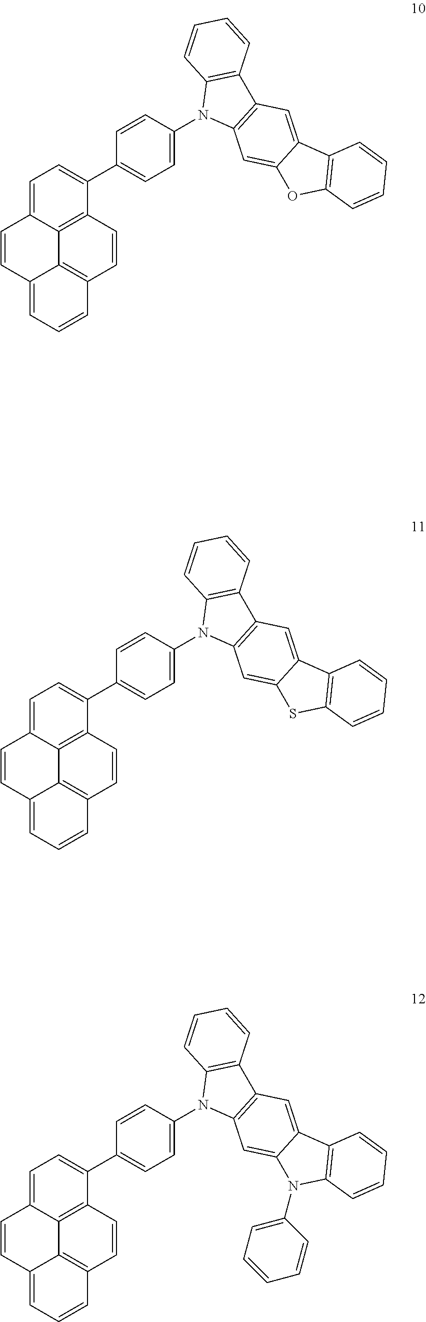 [SMILES: c1cc2ccc3ccc(-c4ccc(-n5c6ccccc6c6cc7c(cc65)oc5ccccc57)cc4)c4ccc(c1)c2c34 ; c1cc2ccc3ccc(-c4ccc(-n5c6ccccc6c6cc7c(cc65)sc5ccccc57)cc4)c4ccc(c1)c2c34 ; c1ccc(-n2c3ccccc3c3cc4c5ccccc5n(-c5ccc(-c6ccc7ccc8cccc9ccc6c7c89)cc5)c4cc32)cc1]

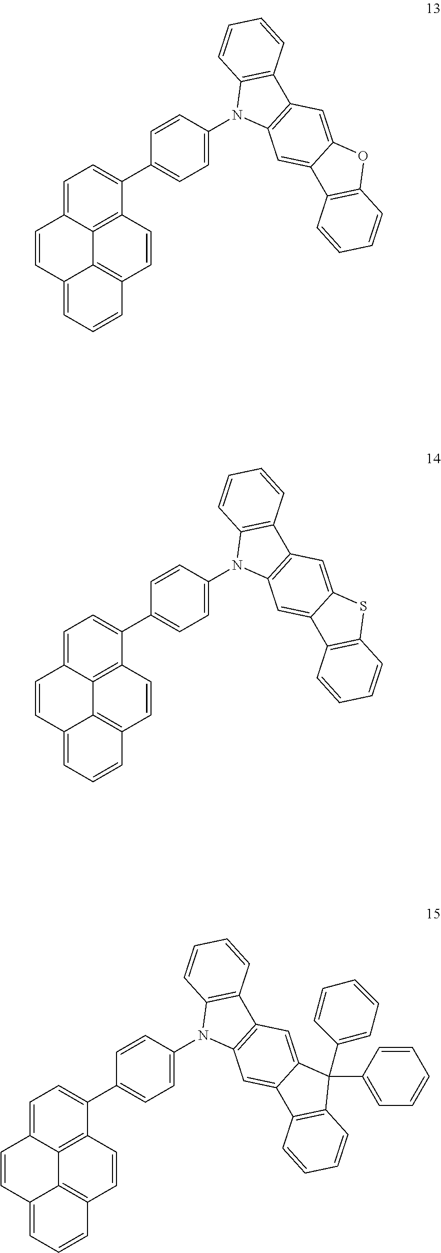 [SMILES: c1cc2ccc3ccc(-c4ccc(-n5c6ccccc6c6cc7oc8ccccc8c7cc65)cc4)c4ccc(c1)c2c34 ; c1cc2ccc3ccc(-c4ccc(-n5c6ccccc6c6cc7sc8ccccc8c7cc65)cc4)c4ccc(c1)c2c34 ; c1ccc(C2(c3ccccc3)c3ccccc3-c3cc4c(cc32)c2ccccc2n4-c2ccc(-c3ccc4ccc5cccc6ccc3c4c56)cc2)cc1]

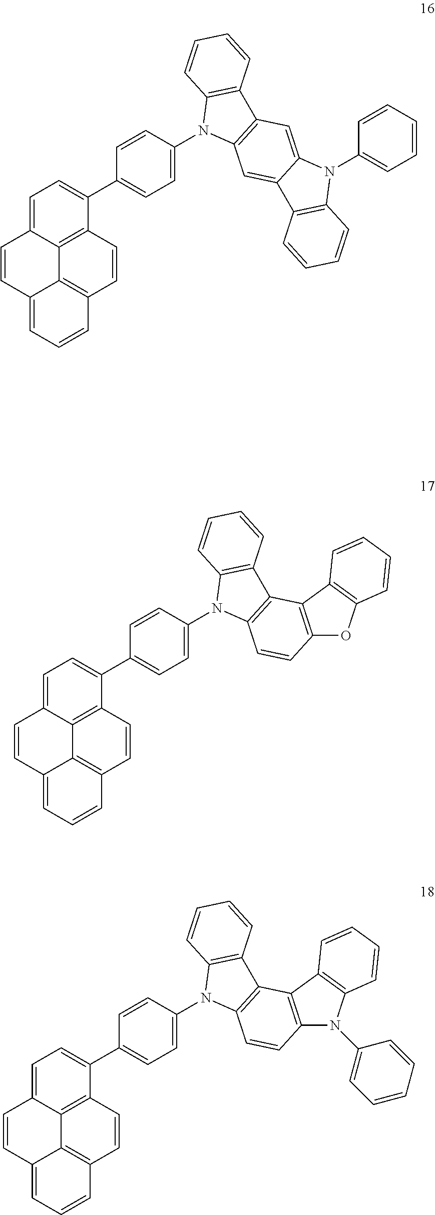 [SMILES: c1ccc(-n2c3ccccc3c3cc4c(cc32)c2ccccc2n4-c2ccc(-c3ccc4ccc5cccc6ccc3c4c56)cc2)cc1 ; c1cc2ccc3ccc(-c4ccc(-n5c6ccccc6c6c7c(ccc65)oc5ccccc57)cc4)c4ccc(c1)c2c34 ; c1ccc(-n2c3ccccc3c3c4c5ccccc5n(-c5ccc(-c6ccc7ccc8cccc9ccc6c7c89)cc5)c4ccc32)cc1]

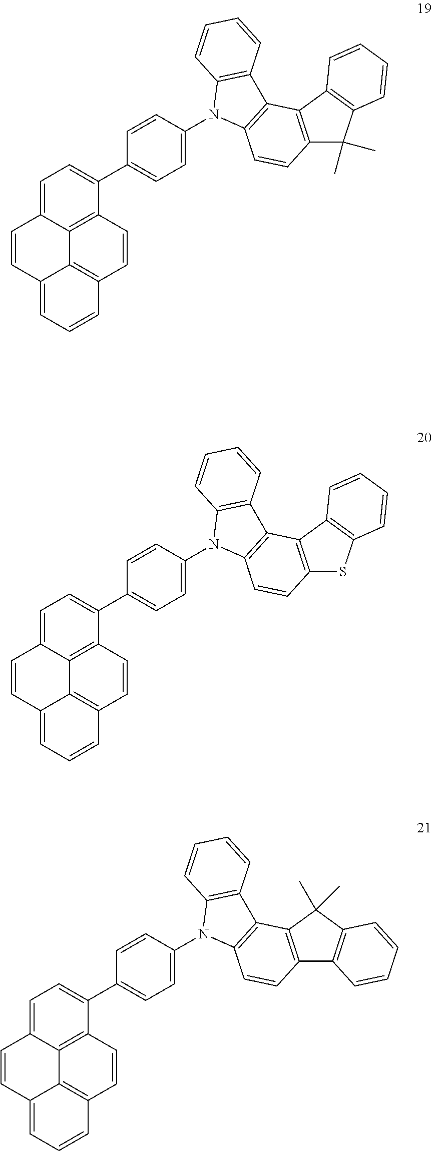 [SMILES: CC1(C)c2ccccc2-c2c1ccc1c2c2ccccc2n1-c1ccc(-c2ccc3ccc4cccc5ccc2c3c45)cc1 ; c1cc2ccc3ccc(-c4ccc(-n5c6ccccc6c6c7c(ccc65)sc5ccccc57)cc4)c4ccc(c1)c2c34 ; CC1(C)c2ccccc2-c2ccc3c(c21)c1ccccc1n3-c1ccc(-c2ccc3ccc4cccc5ccc2c3c45)cc1]

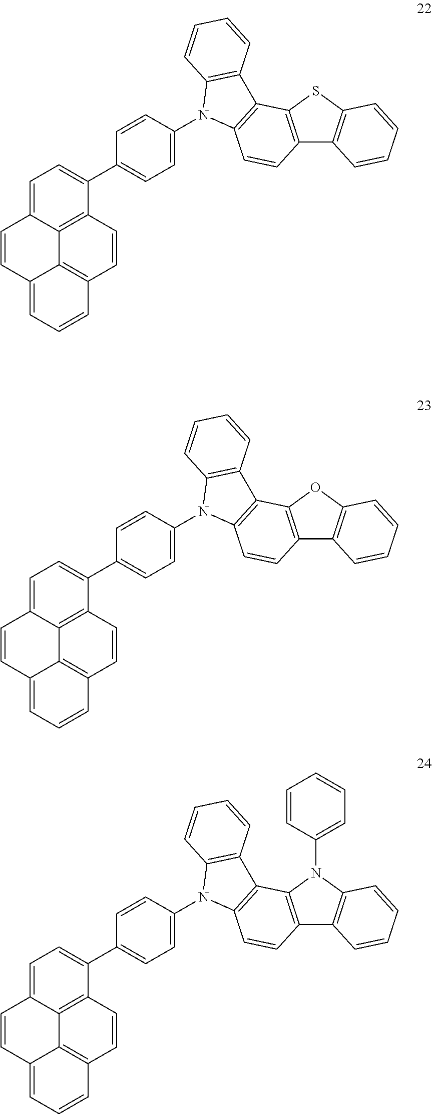 [SMILES: c1cc2ccc3ccc(-c4ccc(-n5c6ccccc6c6c7sc8ccccc8c7ccc65)cc4)c4ccc(c1)c2c34 ; c1cc2ccc3ccc(-c4ccc(-n5c6ccccc6c6c7oc8ccccc8c7ccc65)cc4)c4ccc(c1)c2c34 ; c1ccc(-n2c3ccccc3c3ccc4c(c5ccccc5n4-c4ccc(-c5ccc6ccc7cccc8ccc5c6c78)cc4)c32)cc1]

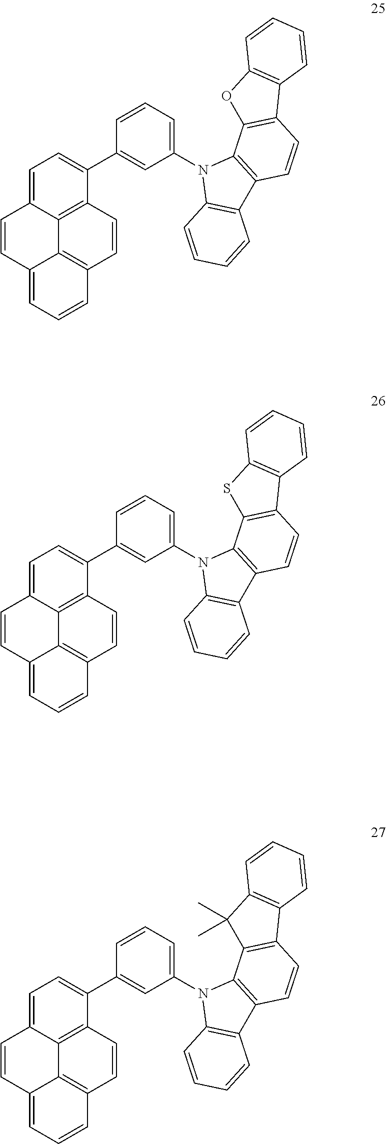 [SMILES: c1cc(-c2ccc3ccc4cccc5ccc2c3c45)cc(-n2c3ccccc3c3ccc4c5ccccc5oc4c32)c1 ; c1cc(-c2ccc3ccc4cccc5ccc2c3c45)cc(-n2c3ccccc3c3ccc4c5ccccc5sc4c32)c1 ; CC1(C)c2ccccc2-c2ccc3c4ccccc4n(-c4cccc(-c5ccc6ccc7cccc8ccc5c6c78)c4)c3c21]

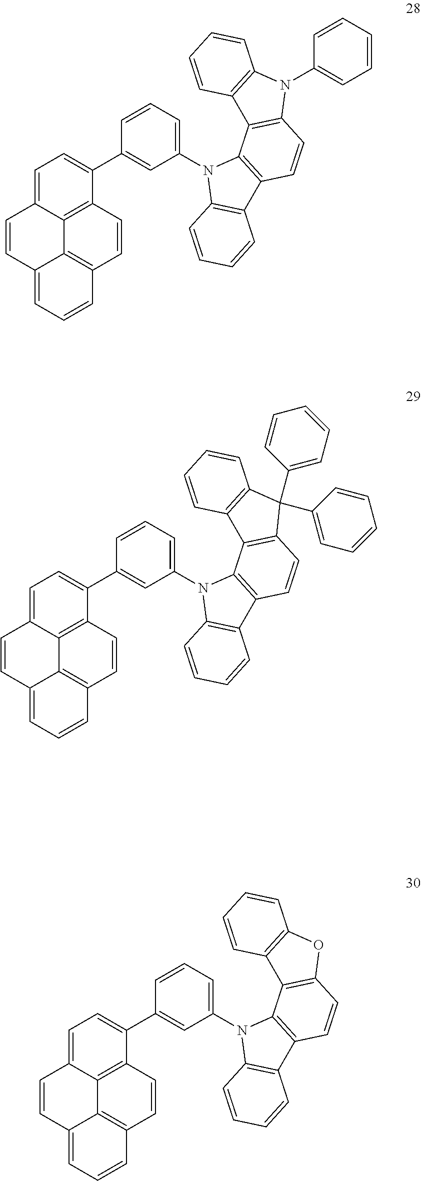 [SMILES: c1ccc(-n2c3ccccc3c3c2ccc2c4ccccc4n(-c4cccc(-c5ccc6ccc7cccc8ccc5c6c78)c4)c23)cc1 ; c1ccc(C2(c3ccccc3)c3ccccc3-c3c2ccc2c4ccccc4n(-c4cccc(-c5ccc6ccc7cccc8ccc5c6c78)c4)c32)cc1 ; c1cc(-c2ccc3ccc4cccc5ccc2c3c45)cc(-n2c3ccccc3c3ccc4oc5ccccc5c4c32)c1]

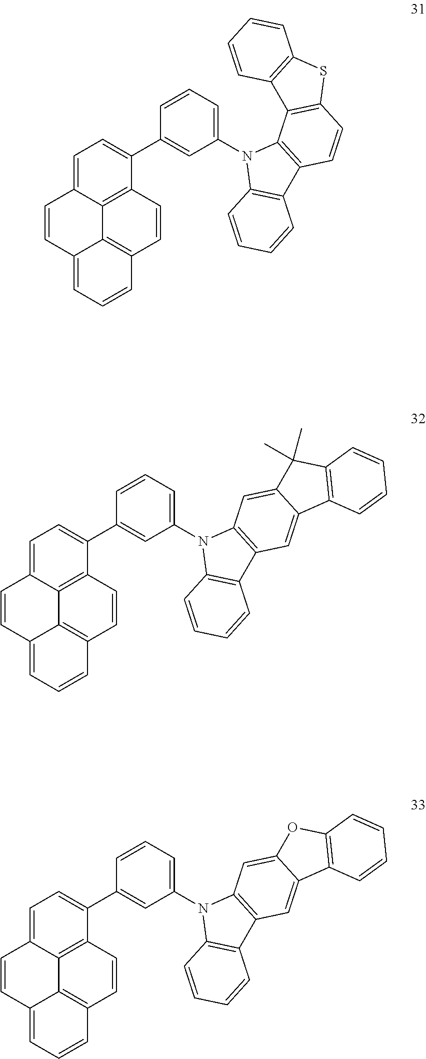 [SMILES: c1cc(-c2ccc3ccc4cccc5ccc2c3c45)cc(-n2c3ccccc3c3ccc4sc5ccccc5c4c32)c1 ; CC1(C)c2ccccc2-c2cc3c4ccccc4n(-c4cccc(-c5ccc6ccc7cccc8ccc5c6c78)c4)c3cc21 ; c1cc(-c2ccc3ccc4cccc5ccc2c3c45)cc(-n2c3ccccc3c3cc4c(cc32)oc2ccccc24)c1]

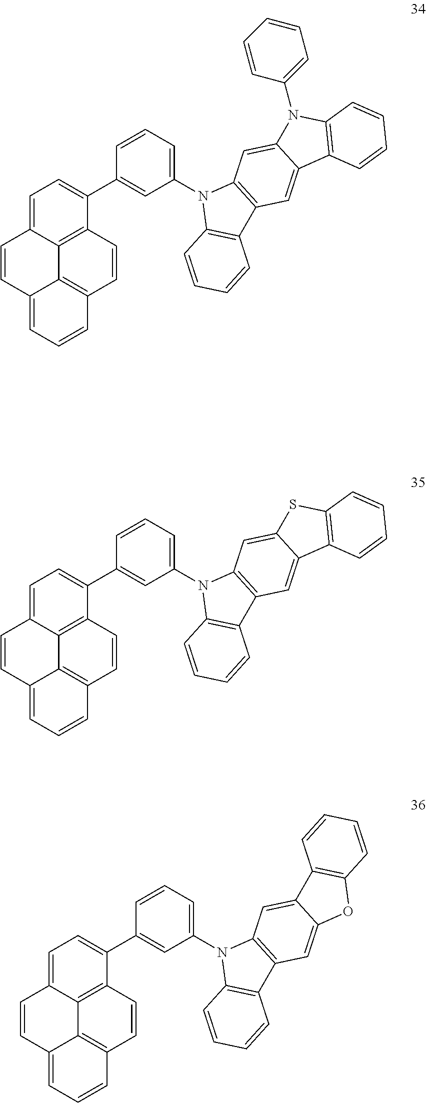 [SMILES: c1ccc(-n2c3ccccc3c3cc4c5ccccc5n(-c5cccc(-c6ccc7ccc8cccc9ccc6c7c89)c5)c4cc32)cc1 ; c1cc(-c2ccc3ccc4cccc5ccc2c3c45)cc(-n2c3ccccc3c3cc4c(cc32)sc2ccccc24)c1 ; c1cc(-c2ccc3ccc4cccc5ccc2c3c45)cc(-n2c3ccccc3c3cc4oc5ccccc5c4cc32)c1]

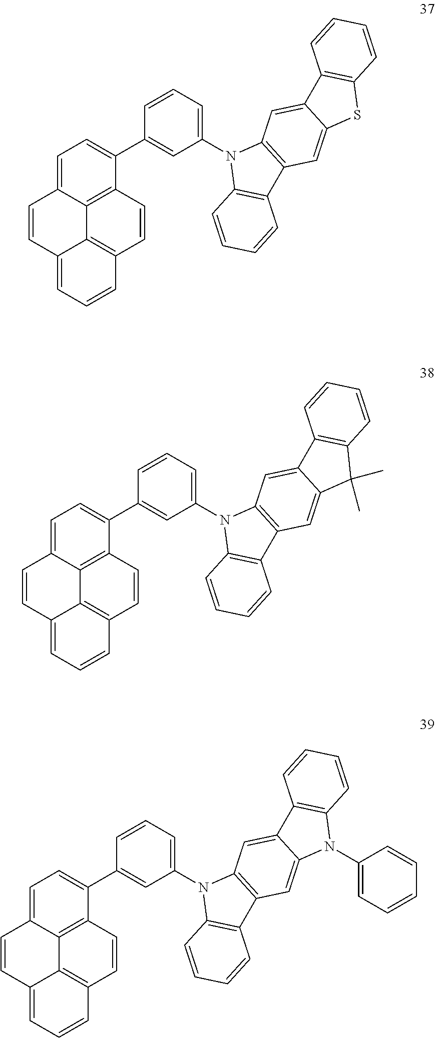 [SMILES: c1cc(-c2ccc3ccc4cccc5ccc2c3c45)cc(-n2c3ccccc3c3cc4sc5ccccc5c4cc32)c1 ; CC1(C)c2ccccc2-c2cc3c(cc21)c1ccccc1n3-c1cccc(-c2ccc3ccc4cccc5ccc2c3c45)c1 ; c1ccc(-n2c3ccccc3c3cc4c(cc32)c2ccccc2n4-c2cccc(-c3ccc4ccc5cccc6ccc3c4c56)c2)cc1]

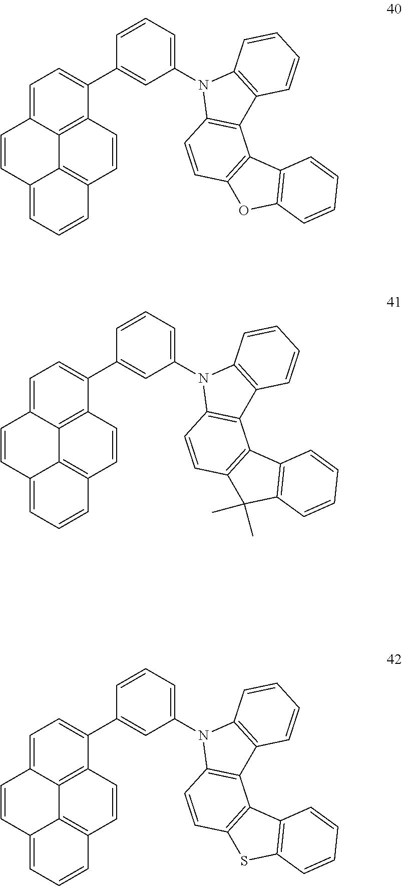 [SMILES: c1cc(-c2ccc3ccc4cccc5ccc2c3c45)cc(-n2c3ccccc3c3c4c(ccc32)oc2ccccc24)c1 ; CC1(C)c2ccccc2-c2c1ccc1c2c2ccccc2n1-c1cccc(-c2ccc3ccc4cccc5ccc2c3c45)c1 ; c1cc(-c2ccc3ccc4cccc5ccc2c3c45)cc(-n2c3ccccc3c3c4c(ccc32)sc2ccccc24)c1]

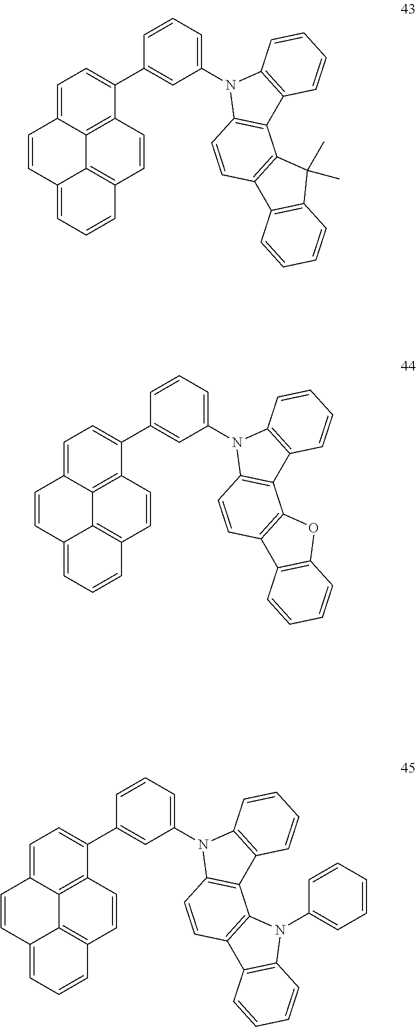 [SMILES: CC1(C)c2ccccc2-c2ccc3c(c21)c1ccccc1n3-c1cccc(-c2ccc3ccc4cccc5ccc2c3c45)c1 ; c1cc(-c2ccc3ccc4cccc5ccc2c3c45)cc(-n2c3ccccc3c3c4oc5ccccc5c4ccc32)c1 ; c1ccc(-n2c3ccccc3c3ccc4c(c5ccccc5n4-c4cccc(-c5ccc6ccc7cccc8ccc5c6c78)c4)c32)cc1]

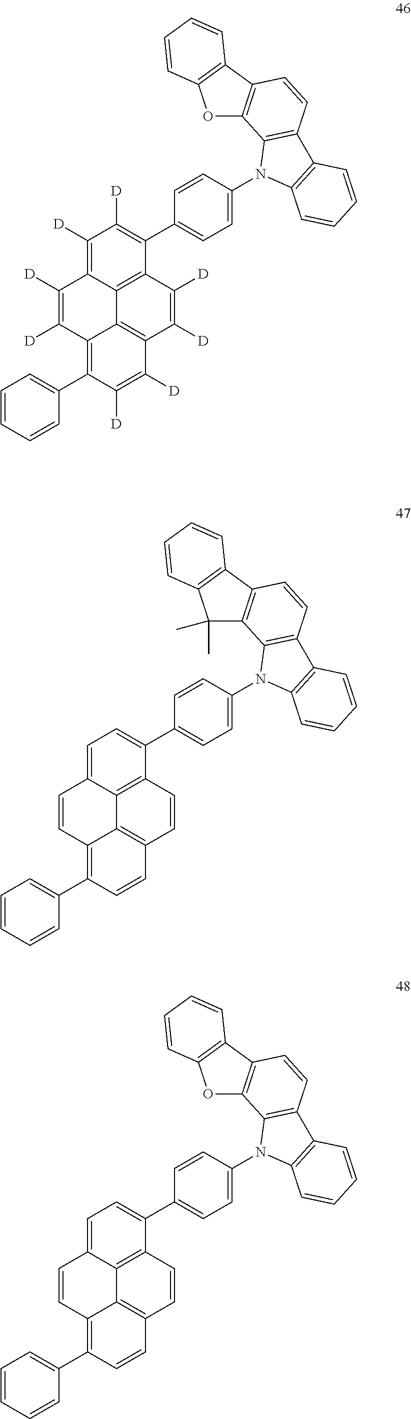 [SMILES: [2H]c1c([2H])c2c([2H])c([2H])c3c(-c4ccc(-n5c6ccccc6c6ccc7c8ccccc8oc7c65)cc4)c([2H])c([2H])c4c([2H])c([2H])c(c1-c1ccccc1)c2c43 ; CC1(C)c2ccccc2-c2ccc3c4ccccc4n(-c4ccc(-c5ccc6ccc7c(-c8ccccc8)ccc8ccc5c6c87)cc4)c3c21 ; c1ccc(-c2ccc3ccc4c(-c5ccc(-n6c7ccccc7c7ccc8c9ccccc9oc8c76)cc5)ccc5ccc2c3c54)cc1]

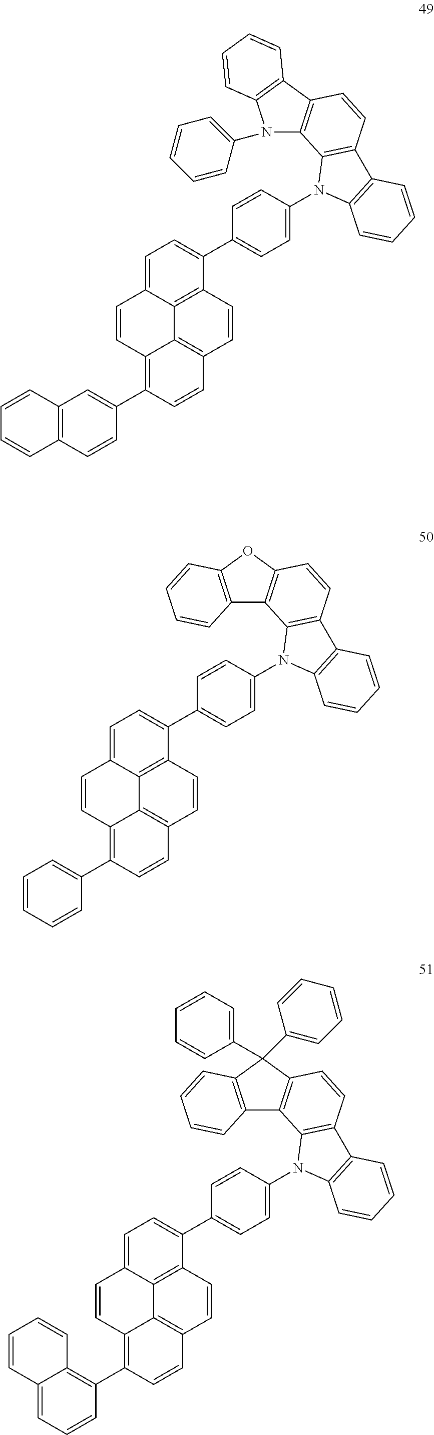 [SMILES: c1ccc(-n2c3ccccc3c3ccc4c5ccccc5n(-c5ccc(-c6ccc7ccc8c(-c9ccc%10ccccc%10c9)ccc9ccc6c7c98)cc5)c4c32)cc1 ; c1ccc(-c2ccc3ccc4c(-c5ccc(-n6c7ccccc7c7ccc8oc9ccccc9c8c76)cc5)ccc5ccc2c3c54)cc1 ; c1ccc(C2(c3ccccc3)c3ccccc3-c3c2ccc2c4ccccc4n(-c4ccc(-c5ccc6ccc7c(-c8cccc9ccccc89)ccc8ccc5c6c87)cc4)c32)cc1]

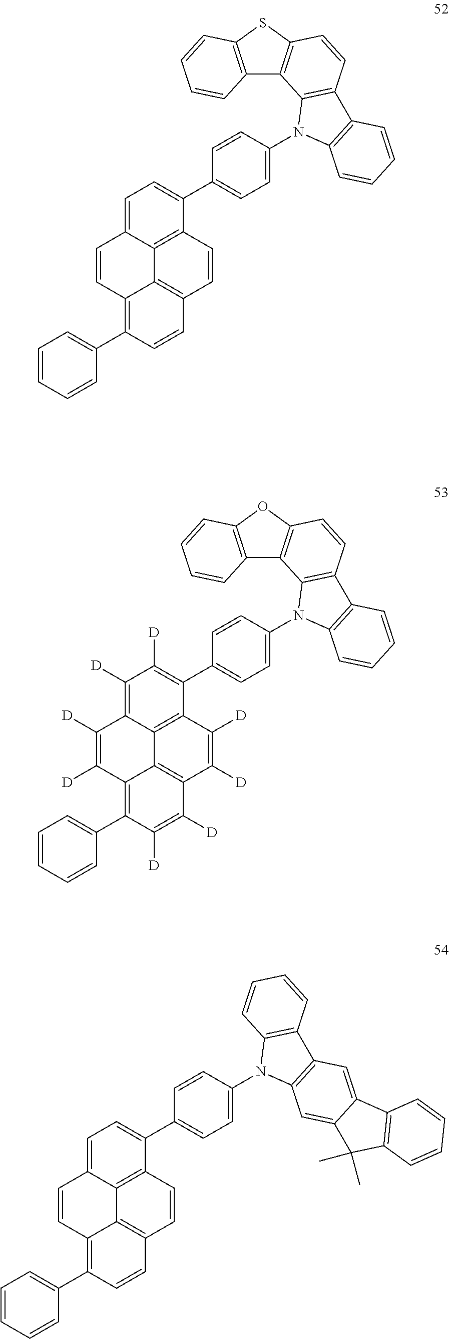 [SMILES: c1ccc(-c2ccc3ccc4c(-c5ccc(-n6c7ccccc7c7ccc8sc9ccccc9c8c76)cc5)ccc5ccc2c3c54)cc1 ; [2H]c1c([2H])c2c([2H])c([2H])c3c(-c4ccc(-n5c6ccccc6c6ccc7oc8ccccc8c7c65)cc4)c([2H])c([2H])c4c([2H])c([2H])c(c1-c1ccccc1)c2c43 ; CC1(C)c2ccccc2-c2cc3c4ccccc4n(-c4ccc(-c5ccc6ccc7c(-c8ccccc8)ccc8ccc5c6c87)cc4)c3cc21]

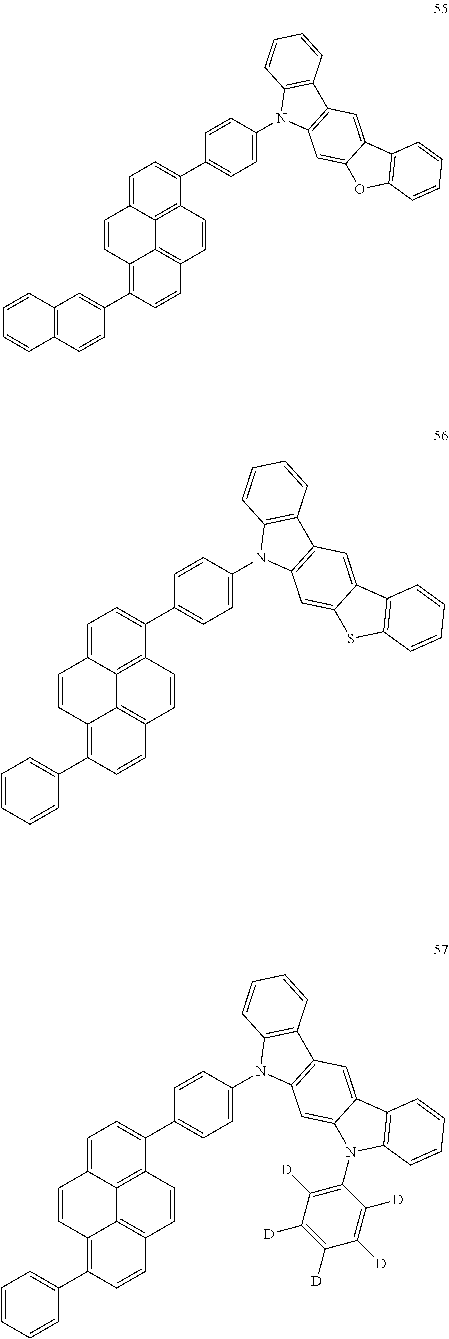 [SMILES: c1ccc2cc(-c3ccc4ccc5c(-c6ccc(-n7c8ccccc8c8cc9c(cc87)oc7ccccc79)cc6)ccc6ccc3c4c65)ccc2c1 ; c1ccc(-c2ccc3ccc4c(-c5ccc(-n6c7ccccc7c7cc8c(cc76)sc6ccccc68)cc5)ccc5ccc2c3c54)cc1 ; [2H]c1c([2H])c([2H])c(-n2c3ccccc3c3cc4c5ccccc5n(-c5ccc(-c6ccc7ccc8c(-c9ccccc9)ccc9ccc6c7c98)cc5)c4cc32)c([2H])c1[2H]]

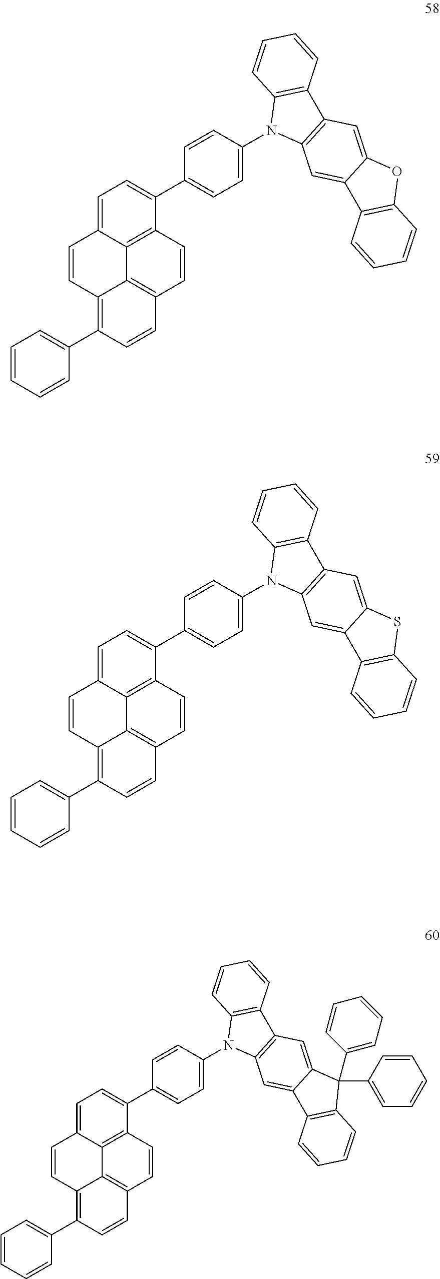 [SMILES: c1ccc(-c2ccc3ccc4c(-c5ccc(-n6c7ccccc7c7cc8oc9ccccc9c8cc76)cc5)ccc5ccc2c3c54)cc1 ; c1ccc(-c2ccc3ccc4c(-c5ccc(-n6c7ccccc7c7cc8sc9ccccc9c8cc76)cc5)ccc5ccc2c3c54)cc1 ; c1ccc(-c2ccc3ccc4c(-c5ccc(-n6c7ccccc7c7cc8c(cc76)-c6ccccc6C8(c6ccccc6)c6ccccc6)cc5)ccc5ccc2c3c54)cc1]

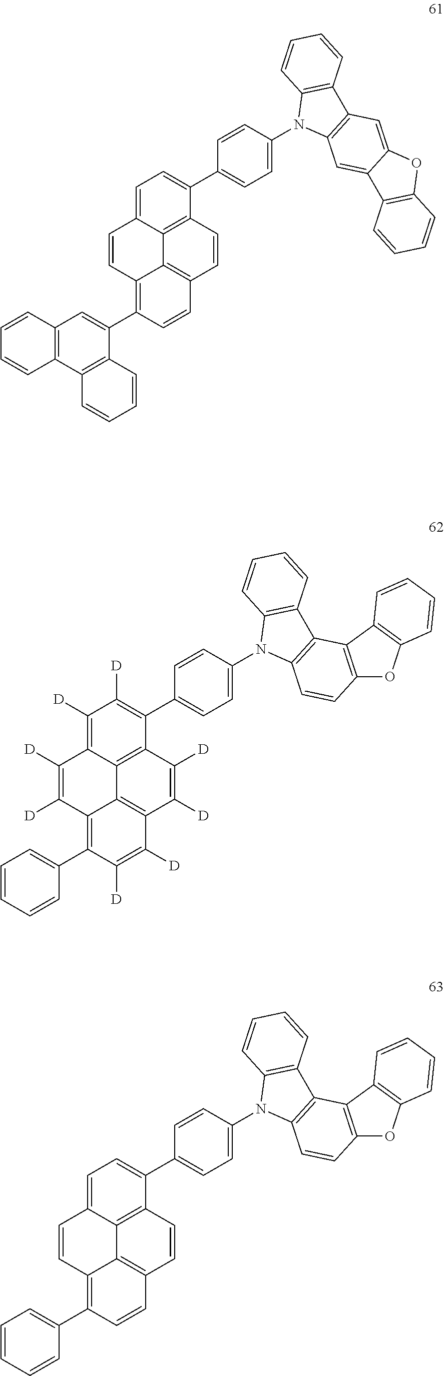 [SMILES: c1ccc2c(c1)cc(-c1ccc3ccc4c(-c5ccc(-n6c7ccccc7c7cc8oc9ccccc9c8cc76)cc5)ccc5ccc1c3c54)c1ccccc12 ; [2H]c1c([2H])c2c([2H])c([2H])c3c(-c4ccc(-n5c6ccccc6c6c7c(ccc65)oc5ccccc57)cc4)c([2H])c([2H])c4c([2H])c([2H])c(c1-c1ccccc1)c2c43 ; c1ccc(-c2ccc3ccc4c(-c5ccc(-n6c7ccccc7c7c8c(ccc76)oc6ccccc68)cc5)ccc5ccc2c3c54)cc1]

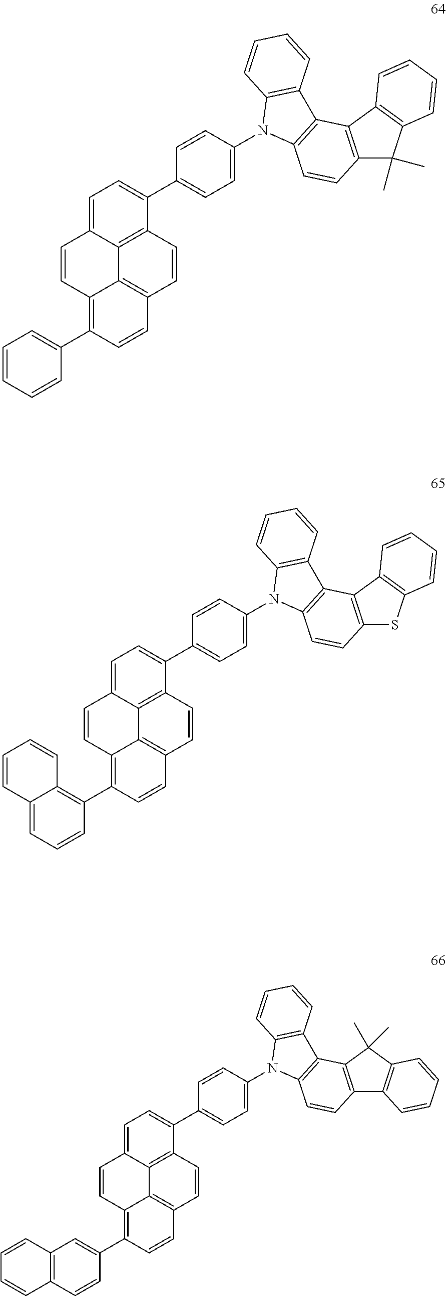 [SMILES: CC1(C)c2ccccc2-c2c1ccc1c2c2ccccc2n1-c1ccc(-c2ccc3ccc4c(-c5ccccc5)ccc5ccc2c3c54)cc1 ; c1ccc2c(-c3ccc4ccc5c(-c6ccc(-n7c8ccccc8c8c9c(ccc87)sc7ccccc79)cc6)ccc6ccc3c4c65)cccc2c1 ; CC1(C)c2ccccc2-c2ccc3c(c21)c1ccccc1n3-c1ccc(-c2ccc3ccc4c(-c5ccc6ccccc6c5)ccc5ccc2c3c54)cc1]

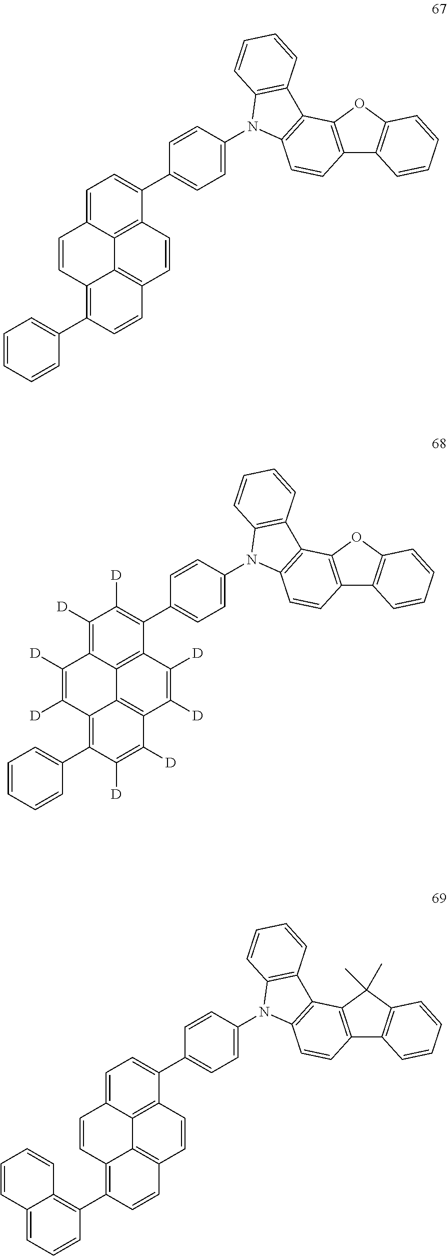 [SMILES: c1ccc(-c2ccc3ccc4c(-c5ccc(-n6c7ccccc7c7c8oc9ccccc9c8ccc76)cc5)ccc5ccc2c3c54)cc1 ; [2H]c1c([2H])c2c([2H])c([2H])c3c(-c4ccc(-n5c6ccccc6c6c7oc8ccccc8c7ccc65)cc4)c([2H])c([2H])c4c([2H])c([2H])c(c1-c1ccccc1)c2c43 ; CC1(C)c2ccccc2-c2ccc3c(c21)c1ccccc1n3-c1ccc(-c2ccc3ccc4c(-c5cccc6ccccc56)ccc5ccc2c3c54)cc1]

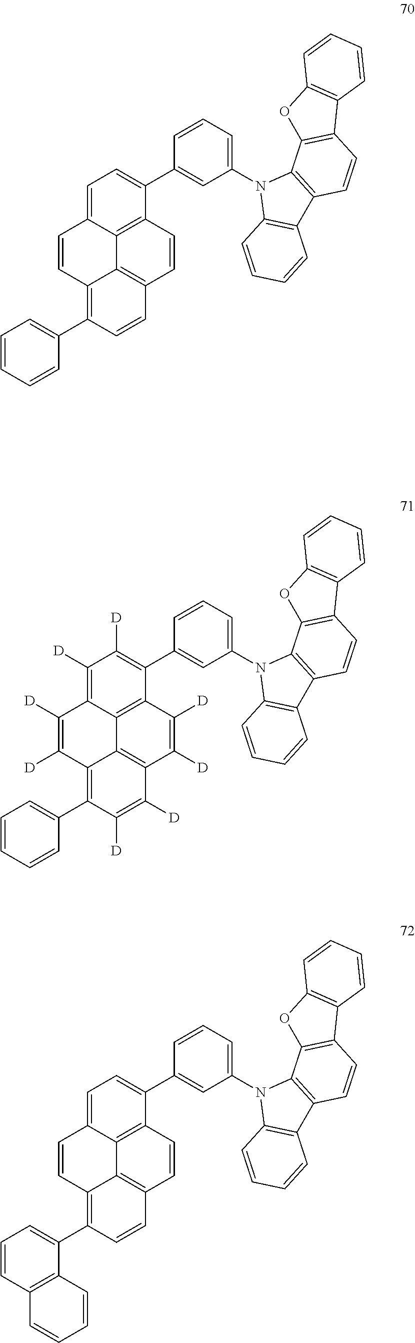 [SMILES: c1ccc(-c2ccc3ccc4c(-c5cccc(-n6c7ccccc7c7ccc8c9ccccc9oc8c76)c5)ccc5ccc2c3c54)cc1 ; [2H]c1c([2H])c2c([2H])c([2H])c3c(-c4cccc(-n5c6ccccc6c6ccc7c8ccccc8oc7c65)c4)c([2H])c([2H])c4c([2H])c([2H])c(c1-c1ccccc1)c2c43 ; c1cc(-c2ccc3ccc4c(-c5cccc6ccccc56)ccc5ccc2c3c54)cc(-n2c3ccccc3c3ccc4c5ccccc5oc4c32)c1]

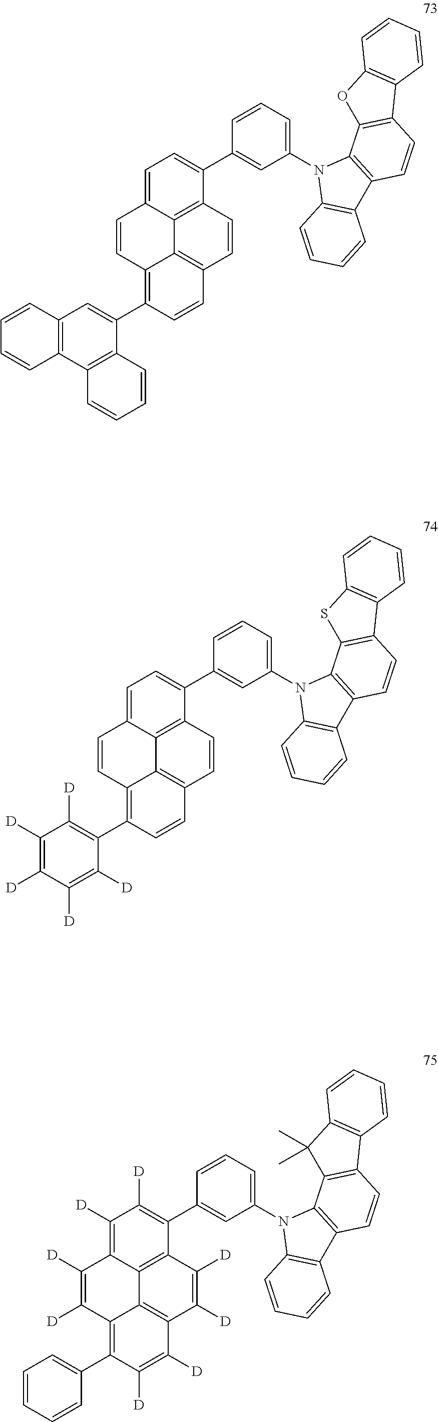 [SMILES: c1cc(-c2ccc3ccc4c(-c5cc6ccccc6c6ccccc56)ccc5ccc2c3c54)cc(-n2c3ccccc3c3ccc4c5ccccc5oc4c32)c1 ; [2H]c1c([2H])c([2H])c(-c2ccc3ccc4c(-c5cccc(-n6c7ccccc7c7ccc8c9ccccc9sc8c76)c5)ccc5ccc2c3c54)c([2H])c1[2H] ; [2H]c1c([2H])c2c([2H])c([2H])c3c(-c4cccc(-n5c6ccccc6c6ccc7c(c65)C(C)(C)c5ccccc5-7)c4)c([2H])c([2H])c4c([2H])c([2H])c(c1-c1ccccc1)c2c43]

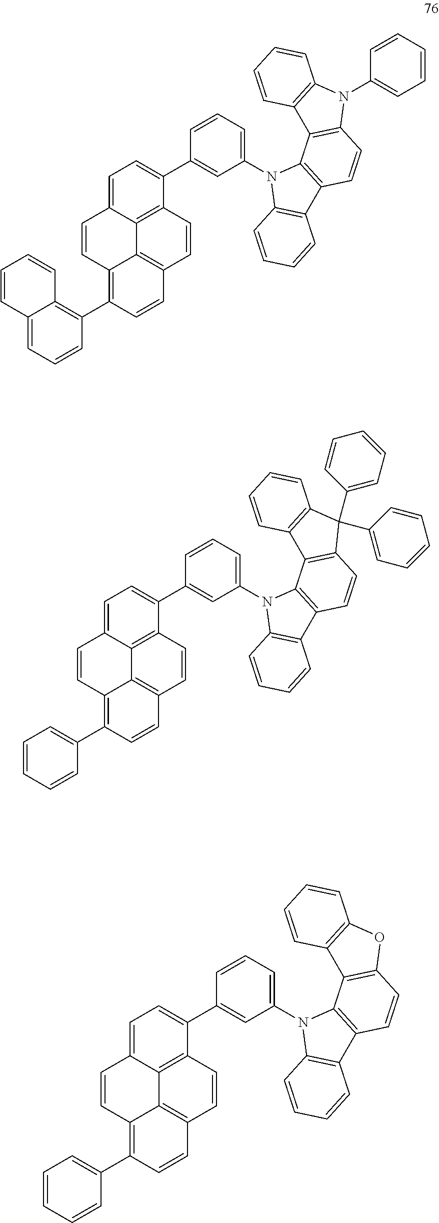 [SMILES: c1ccc(-n2c3ccccc3c3c2ccc2c4ccccc4n(-c4cccc(-c5ccc6ccc7c(-c8cccc9ccccc89)ccc8ccc5c6c87)c4)c23)cc1 ; c1ccc(-c2ccc3ccc4c(-c5cccc(-n6c7ccccc7c7ccc8c(c76)-c6ccccc6C8(c6ccccc6)c6ccccc6)c5)ccc5ccc2c3c54)cc1 ; c1ccc(-c2ccc3ccc4c(-c5cccc(-n6c7ccccc7c7ccc8oc9ccccc9c8c76)c5)ccc5ccc2c3c54)cc1]

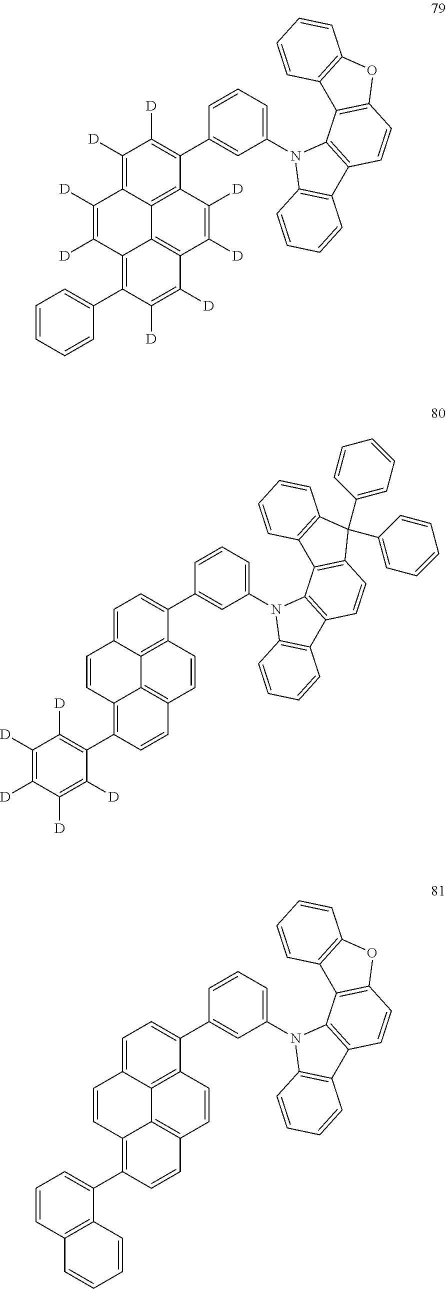 [SMILES: [2H]c1c([2H])c2c([2H])c([2H])c3c(-c4cccc(-n5c6ccccc6c6ccc7oc8ccccc8c7c65)c4)c([2H])c([2H])c4c([2H])c([2H])c(c1-c1ccccc1)c2c43 ; [2H]c1c([2H])c([2H])c(-c2ccc3ccc4c(-c5cccc(-n6c7ccccc7c7ccc8c(c76)-c6ccccc6C8(c6ccccc6)c6ccccc6)c5)ccc5ccc2c3c54)c([2H])c1[2H] ; c1cc(-c2ccc3ccc4c(-c5cccc6ccccc56)ccc5ccc2c3c54)cc(-n2c3ccccc3c3ccc4oc5ccccc5c4c32)c1]

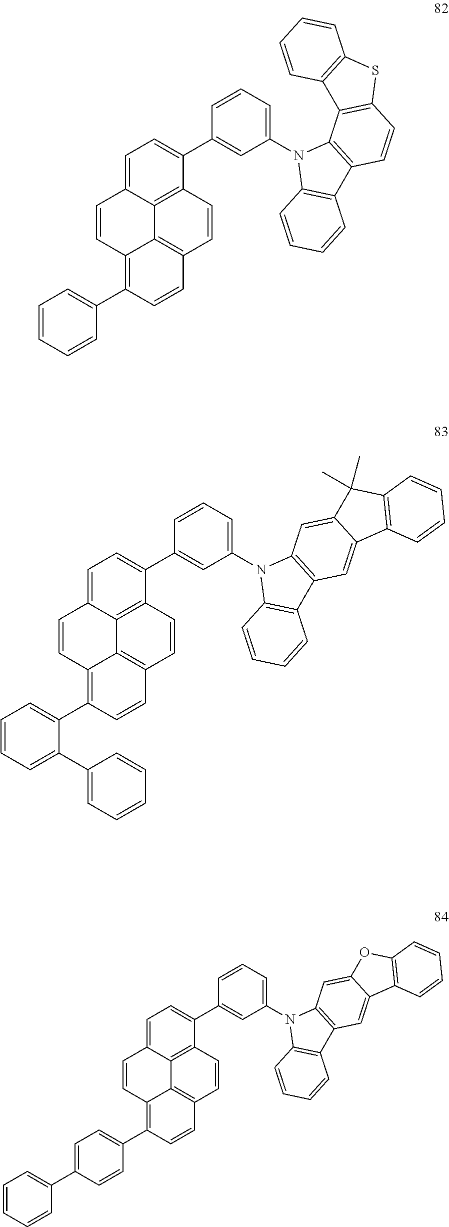 [SMILES: c1ccc(-c2ccc3ccc4c(-c5cccc(-n6c7ccccc7c7ccc8sc9ccccc9c8c76)c5)ccc5ccc2c3c54)cc1 ; CC1(C)c2ccccc2-c2cc3c4ccccc4n(-c4cccc(-c5ccc6ccc7c(-c8ccccc8-c8ccccc8)ccc8ccc5c6c87)c4)c3cc21 ; c1ccc(-c2ccc(-c3ccc4ccc5c(-c6cccc(-n7c8ccccc8c8cc9c(cc87)oc7ccccc79)c6)ccc6ccc3c4c65)cc2)cc1]

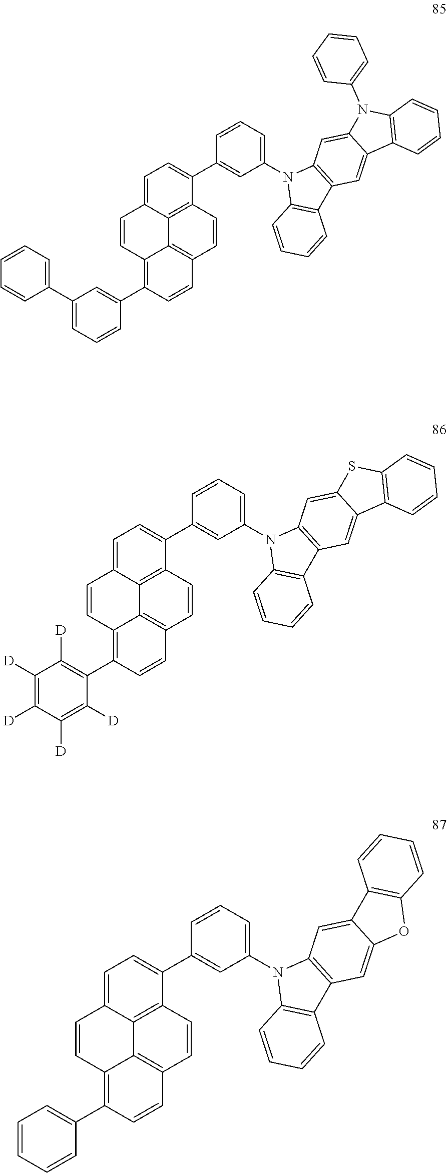 [SMILES: c1ccc(-c2cccc(-c3ccc4ccc5c(-c6cccc(-n7c8ccccc8c8cc9c%10ccccc%10n(-c%10ccccc%10)c9cc87)c6)ccc6ccc3c4c65)c2)cc1 ; [2H]c1c([2H])c([2H])c(-c2ccc3ccc4c(-c5cccc(-n6c7ccccc7c7cc8c(cc76)sc6ccccc68)c5)ccc5ccc2c3c54)c([2H])c1[2H] ; c1ccc(-c2ccc3ccc4c(-c5cccc(-n6c7ccccc7c7cc8oc9ccccc9c8cc76)c5)ccc5ccc2c3c54)cc1]

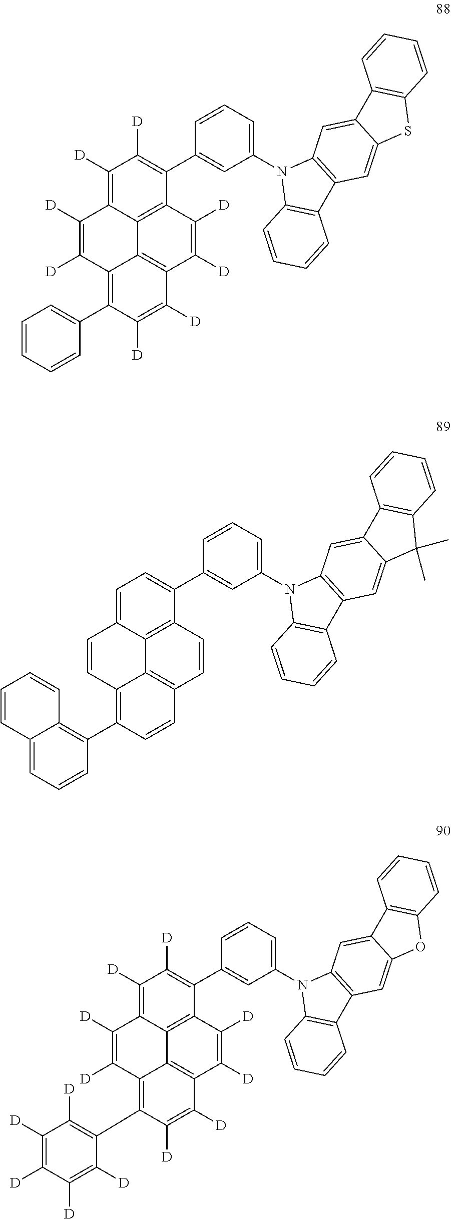 [SMILES: [2H]c1c([2H])c2c([2H])c([2H])c3c(-c4cccc(-n5c6ccccc6c6cc7sc8ccccc8c7cc65)c4)c([2H])c([2H])c4c([2H])c([2H])c(c1-c1ccccc1)c2c43 ; CC1(C)c2ccccc2-c2cc3c(cc21)c1ccccc1n3-c1cccc(-c2ccc3ccc4c(-c5cccc6ccccc56)ccc5ccc2c3c54)c1 ; [2H]c1c([2H])c([2H])c(-c2c([2H])c([2H])c3c([2H])c([2H])c4c(-c5cccc(-n6c7ccccc7c7cc8oc9ccccc9c8cc76)c5)c([2H])c([2H])c5c([2H])c([2H])c2c3c54)c([2H])c1[2H]]

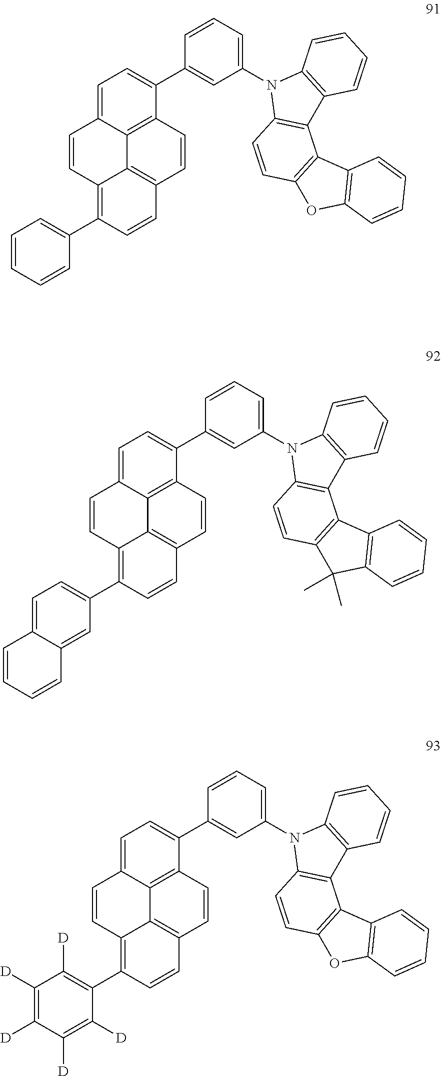 [SMILES: c1ccc(-c2ccc3ccc4c(-c5cccc(-n6c7ccccc7c7c8c(ccc76)oc6ccccc68)c5)ccc5ccc2c3c54)cc1 ; CC1(C)c2ccccc2-c2c1ccc1c2c2ccccc2n1-c1cccc(-c2ccc3ccc4c(-c5ccc6ccccc6c5)ccc5ccc2c3c54)c1 ; [2H]c1c([2H])c([2H])c(-c2ccc3ccc4c(-c5cccc(-n6c7ccccc7c7c8c(ccc76)oc6ccccc68)c5)ccc5ccc2c3c54)c([2H])c1[2H]]

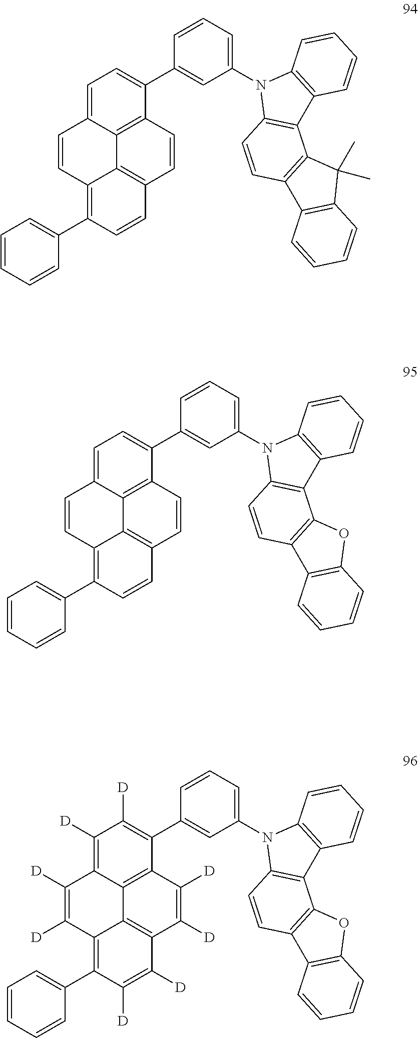 [SMILES: CC1(C)c2ccccc2-c2ccc3c(c21)c1ccccc1n3-c1cccc(-c2ccc3ccc4c(-c5ccccc5)ccc5ccc2c3c54)c1 ; c1ccc(-c2ccc3ccc4c(-c5cccc(-n6c7ccccc7c7c8oc9ccccc9c8ccc76)c5)ccc5ccc2c3c54)cc1 ; [2H]c1c([2H])c2c([2H])c([2H])c3c(-c4cccc(-n5c6ccccc6c6c7oc8ccccc8c7ccc65)c4)c([2H])c([2H])c4c([2H])c([2H])c(c1-c1ccccc1)c2c43]

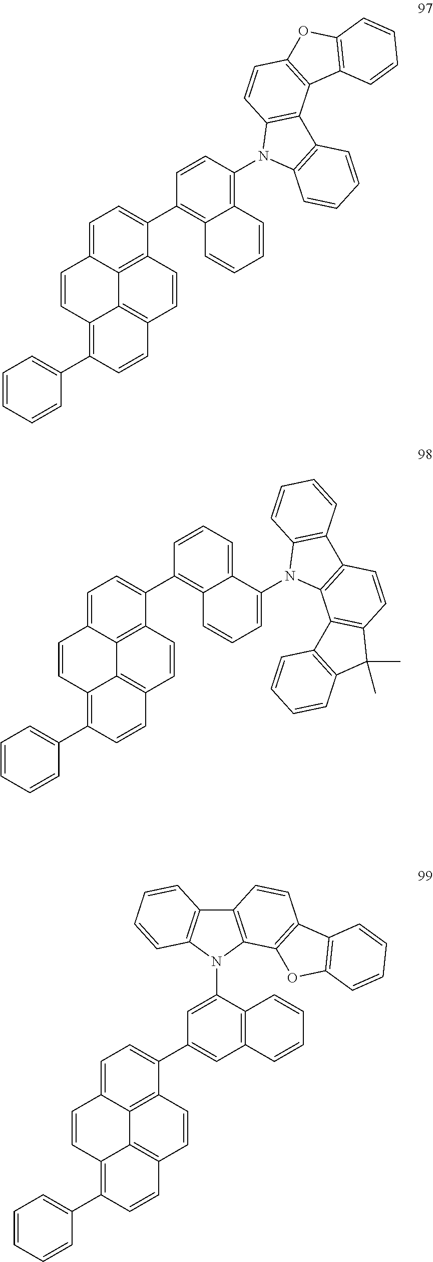 [SMILES: c1ccc(-c2ccc3ccc4c(-c5ccc(-n6c7ccccc7c7c8c(ccc76)oc6ccccc68)c6ccccc56)ccc5ccc2c3c54)cc1 ; CC1(C)c2ccccc2-c2c1ccc1c3ccccc3n(-c3cccc4c(-c5ccc6ccc7c(-c8ccccc8)ccc8ccc5c6c87)cccc34)c21 ; c1ccc(-c2ccc3ccc4c(-c5cc(-n6c7ccccc7c7ccc8c9ccccc9oc8c76)c6ccccc6c5)ccc5ccc2c3c54)cc1]

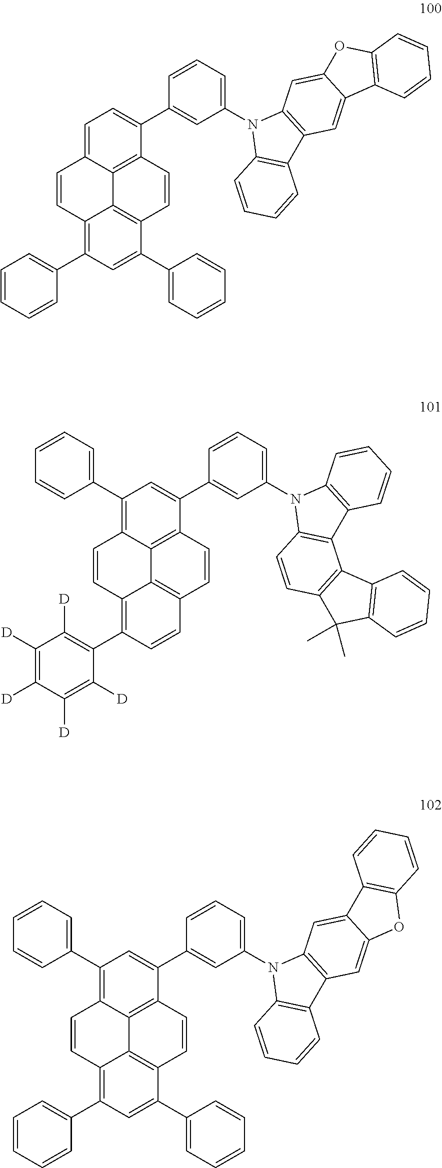 [SMILES: c1ccc(-c2cc(-c3ccccc3)c3ccc4c(-c5cccc(-n6c7ccccc7c7cc8c(cc76)oc6ccccc68)c5)ccc5ccc2c3c54)cc1 ; [2H]c1c([2H])c([2H])c(-c2ccc3ccc4c(-c5cccc(-n6c7ccccc7c7c8c(ccc76)C(C)(C)c6ccccc6-8)c5)cc(-c5ccccc5)c5ccc2c3c54)c([2H])c1[2H] ; c1ccc(-c2cc(-c3ccccc3)c3ccc4c(-c5cccc(-n6c7ccccc7c7cc8oc9ccccc9c8cc76)c5)cc(-c5ccccc5)c5ccc2c3c54)cc1]

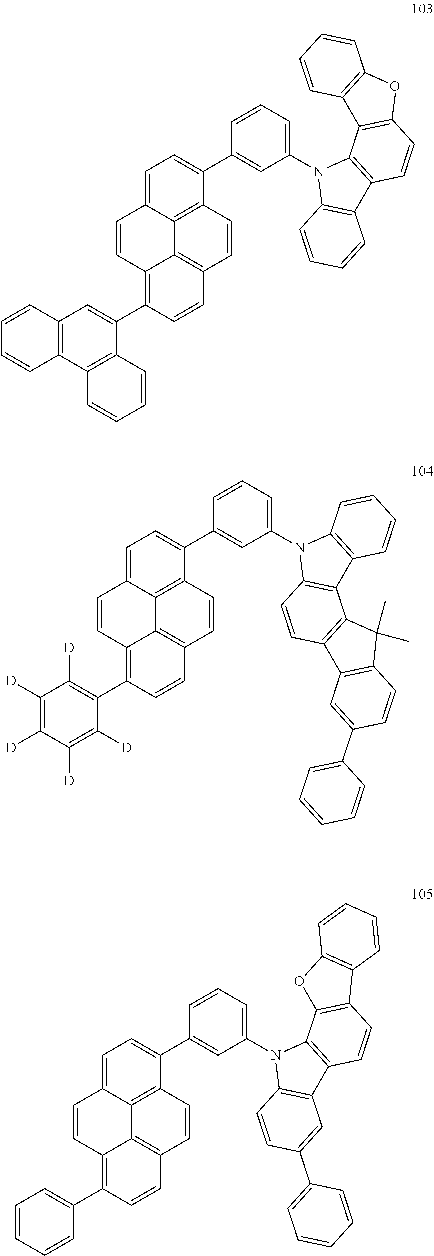 [SMILES: c1cc(-c2ccc3ccc4c(-c5cc6ccccc6c6ccccc56)ccc5ccc2c3c54)cc(-n2c3ccccc3c3ccc4oc5ccccc5c4c32)c1 ; [2H]c1c([2H])c([2H])c(-c2ccc3ccc4c(-c5cccc(-n6c7ccccc7c7c8c(ccc76)-c6cc(-c7ccccc7)ccc6C8(C)C)c5)ccc5ccc2c3c54)c([2H])c1[2H] ; c1ccc(-c2ccc3c(c2)c2ccc4c5ccccc5oc4c2n3-c2cccc(-c3ccc4ccc5c(-c6ccccc6)ccc6ccc3c4c65)c2)cc1]

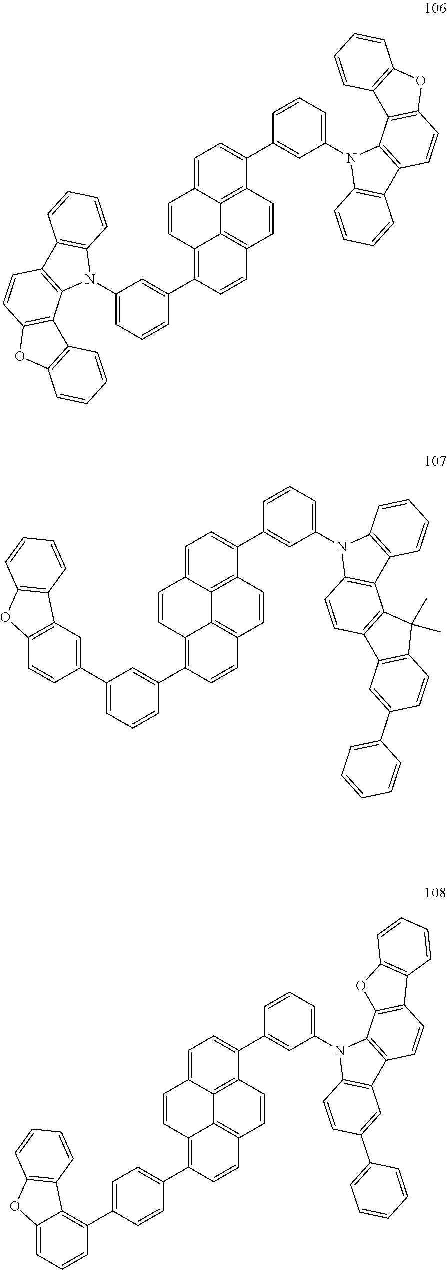 [SMILES: c1cc(-c2ccc3ccc4c(-c5cccc(-n6c7ccccc7c7ccc8oc9ccccc9c8c76)c5)ccc5ccc2c3c54)cc(-n2c3ccccc3c3ccc4oc5ccccc5c4c32)c1 ; CC1(C)c2ccc(-c3ccccc3)cc2-c2ccc3c(c21)c1ccccc1n3-c1cccc(-c2ccc3ccc4c(-c5cccc(-c6ccc7oc8ccccc8c7c6)c5)ccc5ccc2c3c54)c1 ; c1ccc(-c2ccc3c(c2)c2ccc4c5ccccc5oc4c2n3-c2cccc(-c3ccc4ccc5c(-c6ccc(-c7cccc8oc9ccccc9c78)cc6)ccc6ccc3c4c65)c2)cc1]

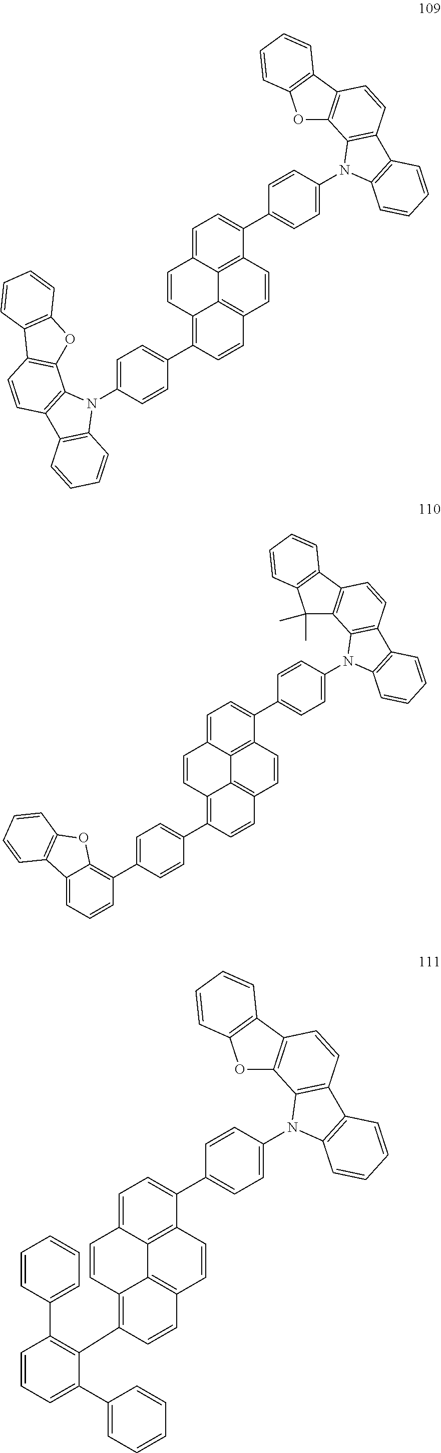 [SMILES: c1ccc2c(c1)oc1c2ccc2c3ccccc3n(-c3ccc(-c4ccc5ccc6c(-c7ccc(-n8c9ccccc9c9ccc%10c%11ccccc%11oc%10c98)cc7)ccc7ccc4c5c76)cc3)c21 ; CC1(C)c2ccccc2-c2ccc3c4ccccc4n(-c4ccc(-c5ccc6ccc7c(-c8ccc(-c9cccc%10c9oc9ccccc9%10)cc8)ccc8ccc5c6c87)cc4)c3c21 ; c1ccc(-c2cccc(-c3ccccc3)c2-c2ccc3ccc4c(-c5ccc(-n6c7ccccc7c7ccc8c9ccccc9oc8c76)cc5)ccc5ccc2c3c54)cc1]

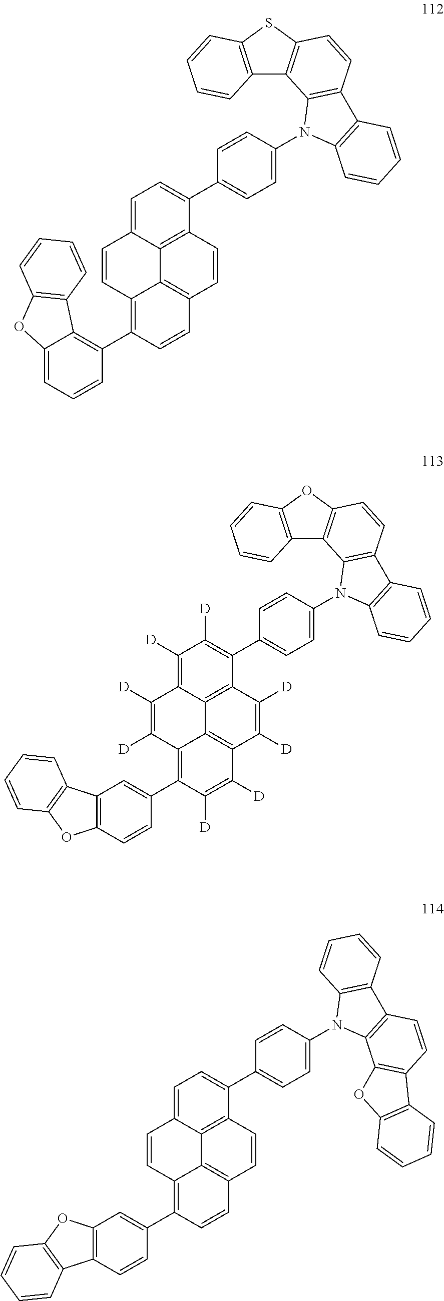 [SMILES: c1ccc2c(c1)oc1cccc(-c3ccc4ccc5c(-c6ccc(-n7c8ccccc8c8ccc9sc%10ccccc%10c9c87)cc6)ccc6ccc3c4c65)c12 ; [2H]c1c([2H])c2c([2H])c([2H])c3c(-c4ccc5oc6ccccc6c5c4)c([2H])c([2H])c4c([2H])c([2H])c(c1-c1ccc(-n5c6ccccc6c6ccc7oc8ccccc8c7c65)cc1)c2c43 ; c1ccc2c(c1)oc1cc(-c3ccc4ccc5c(-c6ccc(-n7c8ccccc8c8ccc9c%10ccccc%10oc9c87)cc6)ccc6ccc3c4c65)ccc12]

9. The compound according to claim 1, wherein the compound represented by Formula 2 is selected from the following compounds 201 to 245:

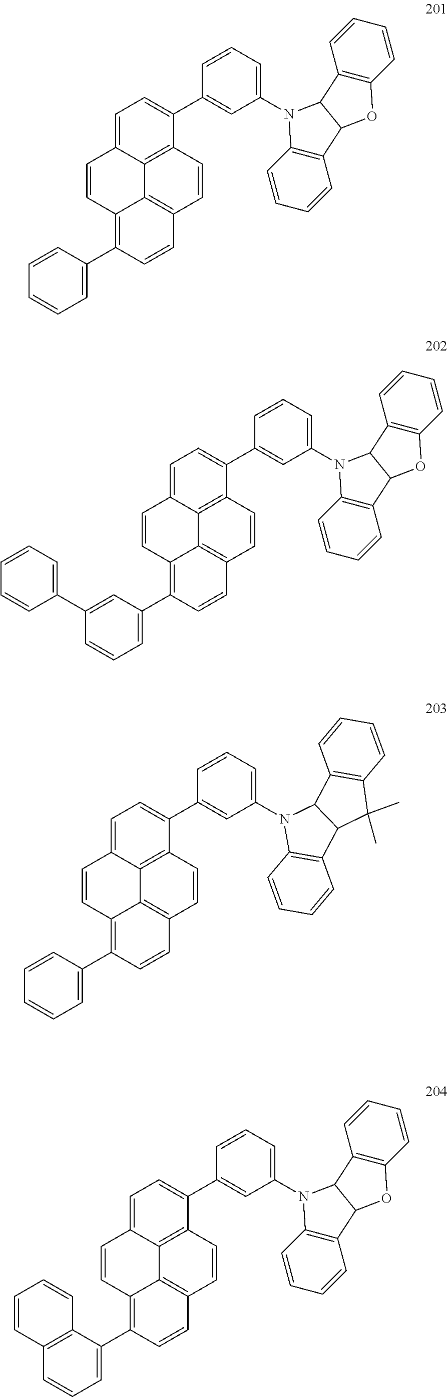 [SMILES: c1ccc(-c2ccc3ccc4c(-c5cccc(N6c7ccccc7C7Oc8ccccc8C76)c5)ccc5ccc2c3c54)cc1 ; c1ccc(-c2cccc(-c3ccc4ccc5c(-c6cccc(N7c8ccccc8C8Oc9ccccc9C87)c6)ccc6ccc3c4c65)c2)cc1 ; CC1(C)c2ccccc2C2C1c1ccccc1N2c1cccc(-c2ccc3ccc4c(-c5ccccc5)ccc5ccc2c3c54)c1 ; c1cc(-c2ccc3ccc4c(-c5cccc6ccccc56)ccc5ccc2c3c54)cc(N2c3ccccc3C3Oc4ccccc4C32)c1]

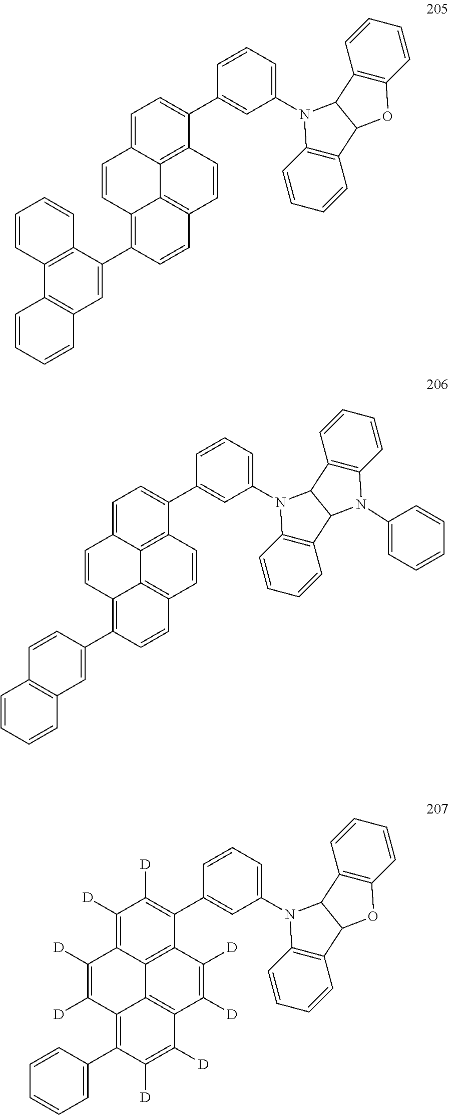 [SMILES: c1cc(-c2ccc3ccc4c(-c5cc6ccccc6c6ccccc56)ccc5ccc2c3c54)cc(N2c3ccccc3C3Oc4ccccc4C32)c1 ; c1ccc(N2c3ccccc3C3C2c2ccccc2N3c2cccc(-c3ccc4ccc5c(-c6ccc7ccccc7c6)ccc6ccc3c4c65)c2)cc1 ; [2H]c1c([2H])c2c([2H])c([2H])c3c(-c4cccc(N5c6ccccc6C6Oc7ccccc7C65)c4)c([2H])c([2H])c4c([2H])c([2H])c(c1-c1ccccc1)c2c43]

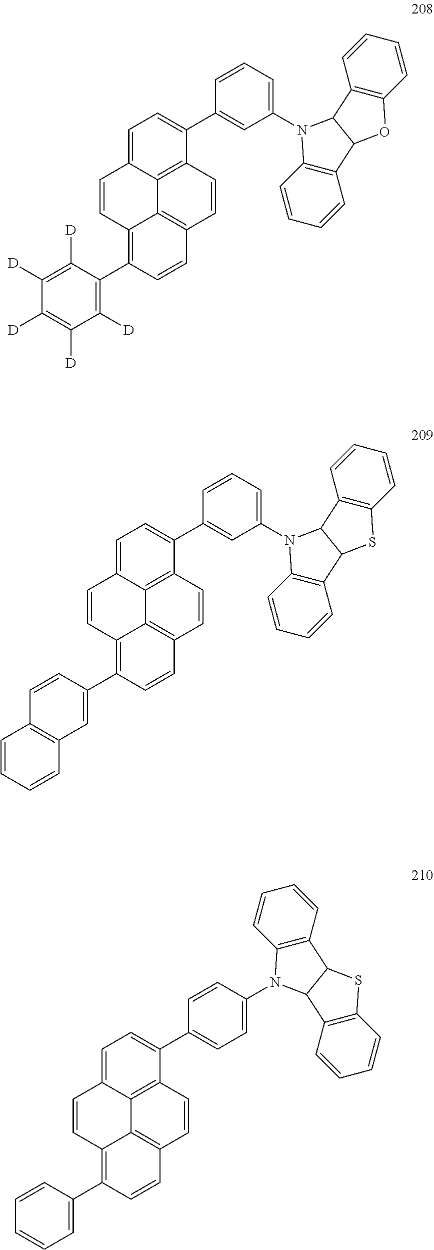 [SMILES: [2H]c1c([2H])c([2H])c(-c2ccc3ccc4c(-c5cccc(N6c7ccccc7C7Oc8ccccc8C76)c5)ccc5ccc2c3c54)c([2H])c1[2H] ; c1cc(-c2ccc3ccc4c(-c5ccc6ccccc6c5)ccc5ccc2c3c54)cc(N2c3ccccc3C3Sc4ccccc4C32)c1 ; c1ccc(-c2ccc3ccc4c(-c5ccc(N6c7ccccc7C7Sc8ccccc8C76)cc5)ccc5ccc2c3c54)cc1]

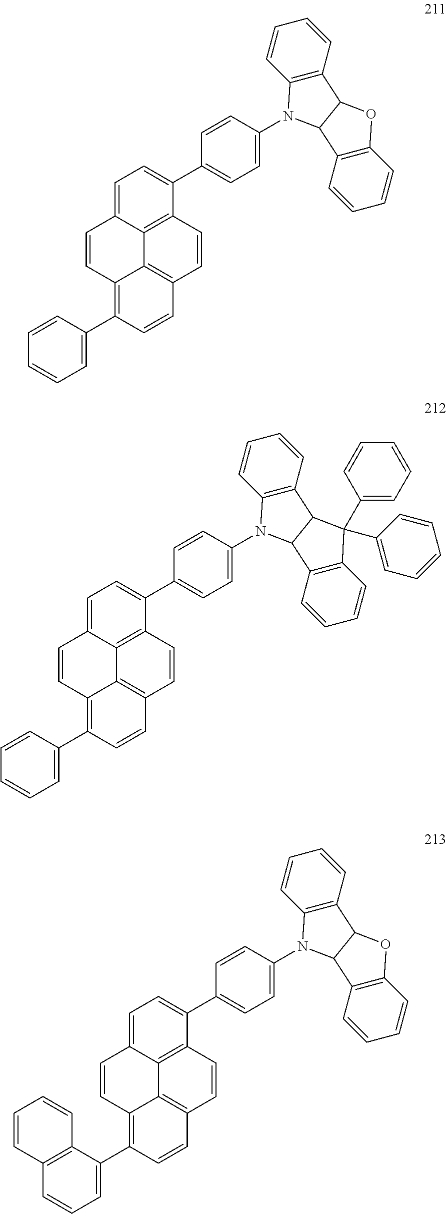 [SMILES: c1ccc(-c2ccc3ccc4c(-c5ccc(N6c7ccccc7C7Oc8ccccc8C76)cc5)ccc5ccc2c3c54)cc1 ; c1ccc(-c2ccc3ccc4c(-c5ccc(N6c7ccccc7C7C6c6ccccc6C7(c6ccccc6)c6ccccc6)cc5)ccc5ccc2c3c54)cc1 ; c1ccc2c(c1)OC1c3ccccc3N(c3ccc(-c4ccc5ccc6c(-c7cccc8ccccc78)ccc7ccc4c5c76)cc3)C21]

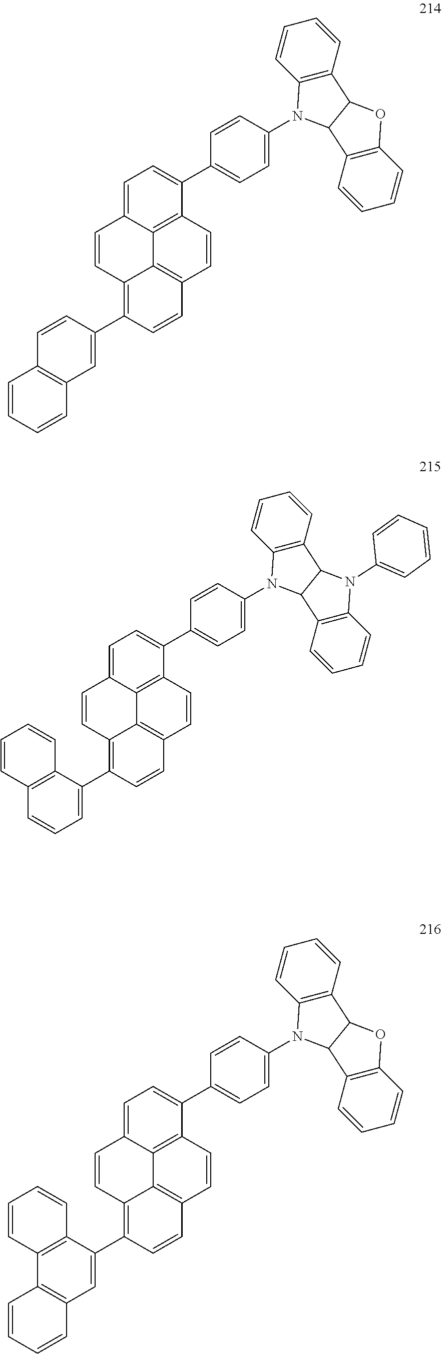 [SMILES: c1ccc2c(c1)OC1c3ccccc3N(c3ccc(-c4ccc5ccc6c(-c7ccc8ccccc8c7)ccc7ccc4c5c76)cc3)C21 ; c1ccc(N2c3ccccc3C3C2c2ccccc2N3c2ccc(-c3ccc4ccc5c(-c6cccc7ccccc67)ccc6ccc3c4c65)cc2)cc1 ; c1ccc2c(c1)OC1c3ccccc3N(c3ccc(-c4ccc5ccc6c(-c7cc8ccccc8c8ccccc78)ccc7ccc4c5c76)cc3)C21]

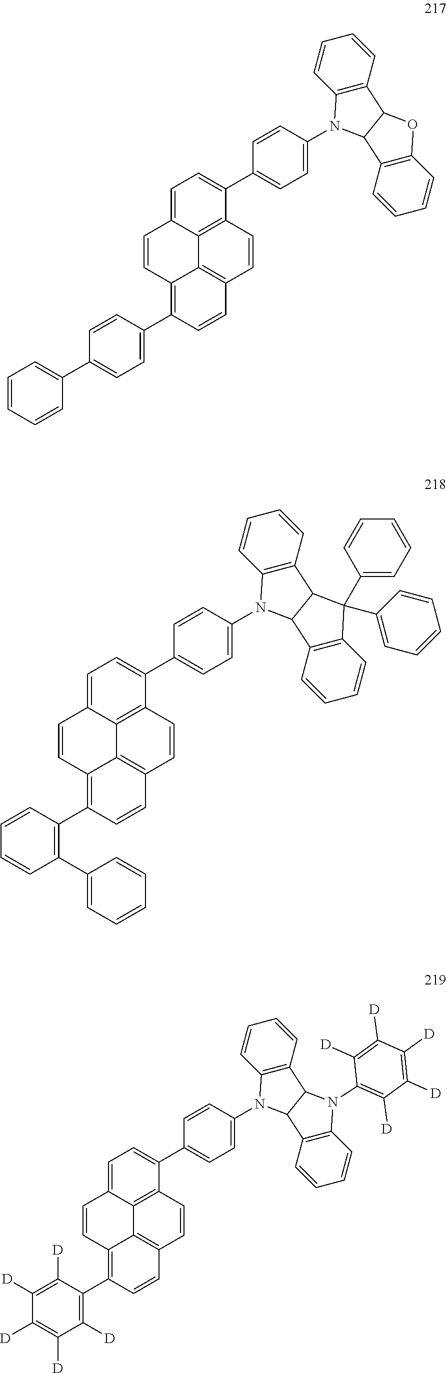 [SMILES: c1ccc(-c2ccc(-c3ccc4ccc5c(-c6ccc(N7c8ccccc8C8Oc9ccccc9C87)cc6)ccc6ccc3c4c65)cc2)cc1 ; c1ccc(-c2ccccc2-c2ccc3ccc4c(-c5ccc(N6c7ccccc7C7C6c6ccccc6C7(c6ccccc6)c6ccccc6)cc5)ccc5ccc2c3c54)cc1 ; [2H]c1c([2H])c([2H])c(-c2ccc3ccc4c(-c5ccc(N6c7ccccc7C7C6c6ccccc6N7c6c([2H])c([2H])c([2H])c([2H])c6[2H])cc5)ccc5ccc2c3c54)c([2H])c1[2H]]

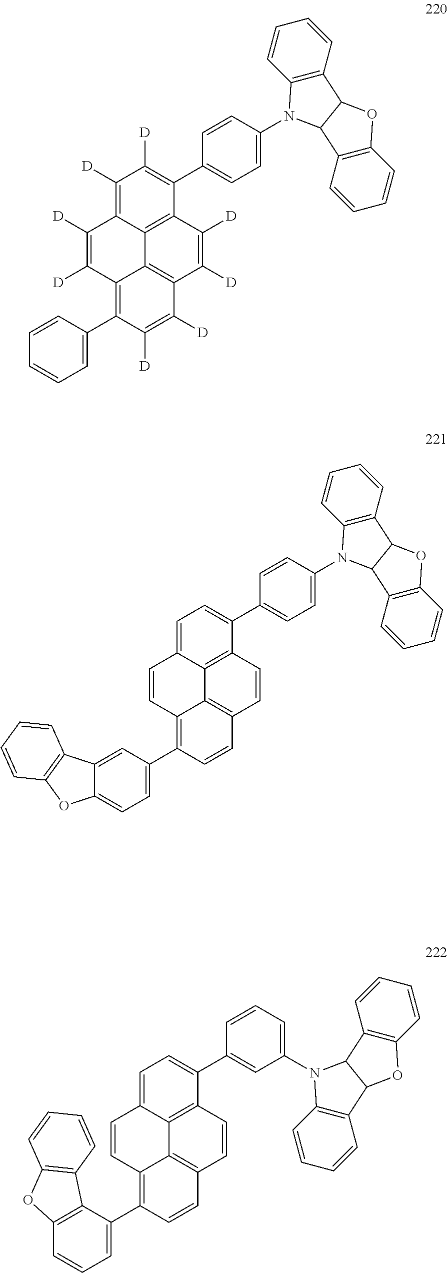 [SMILES: [2H]c1c([2H])c2c([2H])c([2H])c3c(-c4ccc(N5c6ccccc6C6Oc7ccccc7C65)cc4)c([2H])c([2H])c4c([2H])c([2H])c(c1-c1ccccc1)c2c43 ; c1ccc2c(c1)OC1c3ccccc3N(c3ccc(-c4ccc5ccc6c(-c7ccc8oc9ccccc9c8c7)ccc7ccc4c5c76)cc3)C21 ; c1cc(-c2ccc3ccc4c(-c5cccc6oc7ccccc7c56)ccc5ccc2c3c54)cc(N2c3ccccc3C3Oc4ccccc4C32)c1]

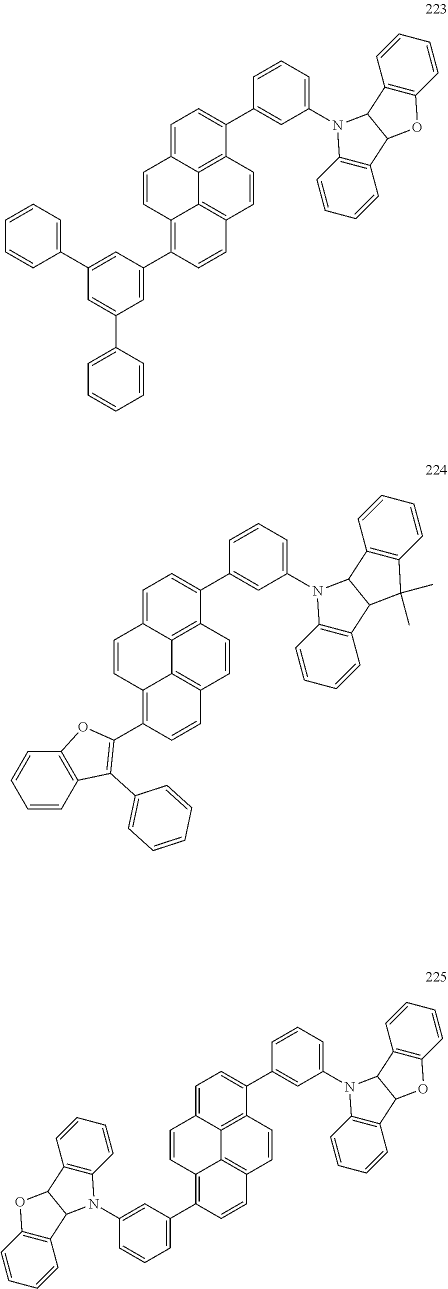 [SMILES: c1ccc(-c2cc(-c3ccccc3)cc(-c3ccc4ccc5c(-c6cccc(N7c8ccccc8C8Oc9ccccc9C87)c6)ccc6ccc3c4c65)c2)cc1 ; CC1(C)c2ccccc2C2C1c1ccccc1N2c1cccc(-c2ccc3ccc4c(-c5oc6ccccc6c5-c5ccccc5)ccc5ccc2c3c54)c1 ; c1cc(-c2ccc3ccc4c(-c5cccc(N6c7ccccc7C7Oc8ccccc8C76)c5)ccc5ccc2c3c54)cc(N2c3ccccc3C3Oc4ccccc4C32)c1]

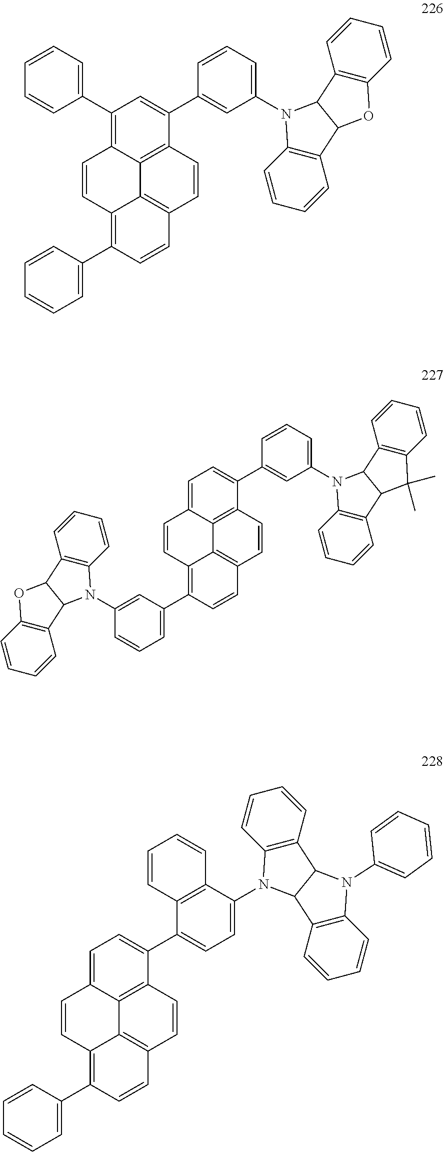 [SMILES: c1ccc(-c2ccc3ccc4c(-c5cccc(N6c7ccccc7C7Oc8ccccc8C76)c5)cc(-c5ccccc5)c5ccc2c3c54)cc1 ; CC1(C)c2ccccc2C2C1c1ccccc1N2c1cccc(-c2ccc3ccc4c(-c5cccc(N6c7ccccc7C7Oc8ccccc8C76)c5)ccc5ccc2c3c54)c1 ; c1ccc(-c2ccc3ccc4c(-c5ccc(N6c7ccccc7C7C6c6ccccc6N7c6ccccc6)c6ccccc56)ccc5ccc2c3c54)cc1]

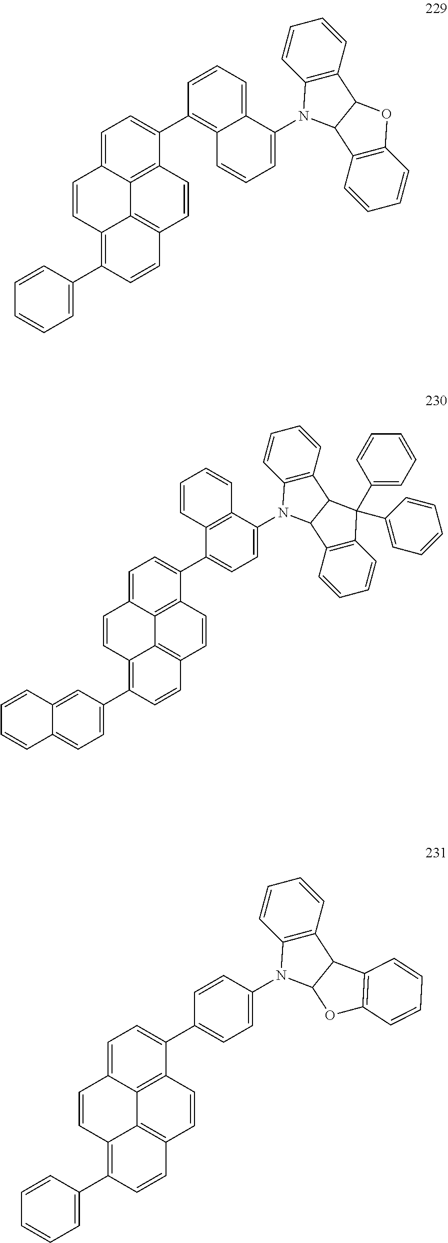 [SMILES: c1ccc(-c2ccc3ccc4c(-c5cccc6c(N7c8ccccc8C8Oc9ccccc9C87)cccc56)ccc5ccc2c3c54)cc1 ; c1ccc(C2(c3ccccc3)c3ccccc3C3C2c2ccccc2N3c2ccc(-c3ccc4ccc5c(-c6ccc7ccccc7c6)ccc6ccc3c4c65)c3ccccc23)cc1 ; c1ccc(-c2ccc3ccc4c(-c5ccc(N6c7ccccc7C7c8ccccc8OC76)cc5)ccc5ccc2c3c54)cc1]

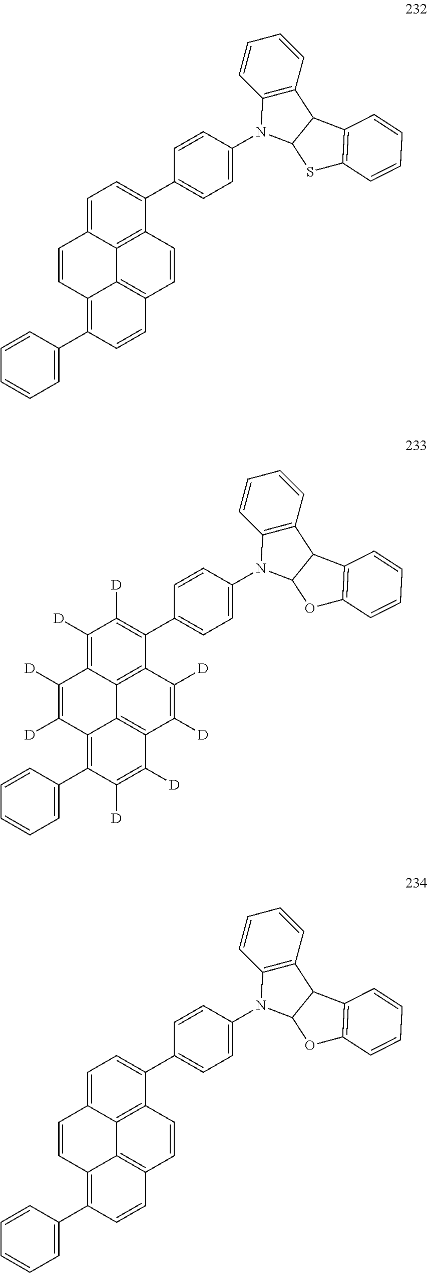 [SMILES: c1ccc(-c2ccc3ccc4c(-c5ccc(N6c7ccccc7C7c8ccccc8SC76)cc5)ccc5ccc2c3c54)cc1 ; [2H]c1c([2H])c2c([2H])c([2H])c3c(-c4ccc(N5c6ccccc6C6c7ccccc7OC65)cc4)c([2H])c([2H])c4c([2H])c([2H])c(c1-c1ccccc1)c2c43 ; c1ccc(-c2ccc3ccc4c(-c5ccc(N6c7ccccc7C7c8ccccc8OC76)cc5)ccc5ccc2c3c54)cc1]

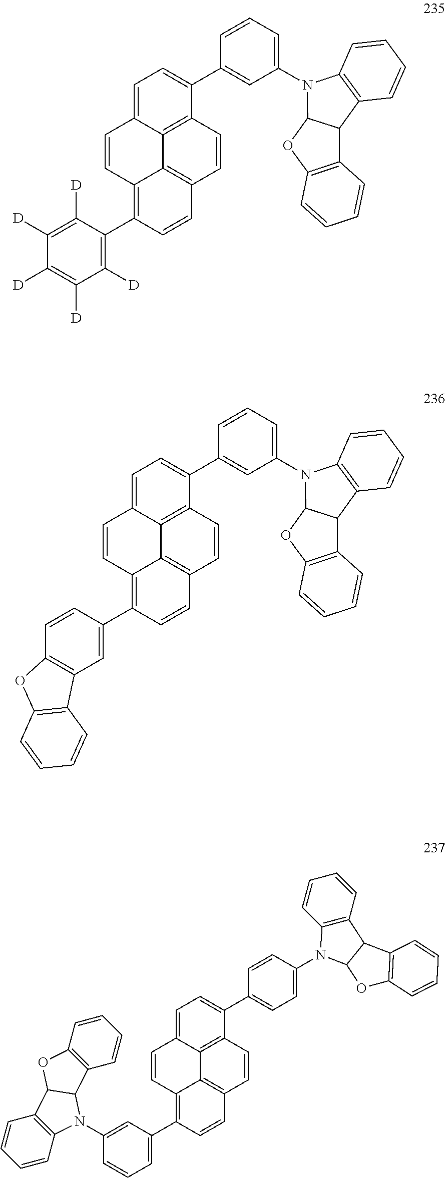 [SMILES: [2H]c1c([2H])c([2H])c(-c2ccc3ccc4c(-c5cccc(N6c7ccccc7C7c8ccccc8OC76)c5)ccc5ccc2c3c54)c([2H])c1[2H] ; c1cc(-c2ccc3ccc4c(-c5ccc6oc7ccccc7c6c5)ccc5ccc2c3c54)cc(N2c3ccccc3C3c4ccccc4OC32)c1 ; c1cc(-c2ccc3ccc4c(-c5ccc(N6c7ccccc7C7c8ccccc8OC76)cc5)ccc5ccc2c3c54)cc(N2c3ccccc3C3Oc4ccccc4C32)c1]

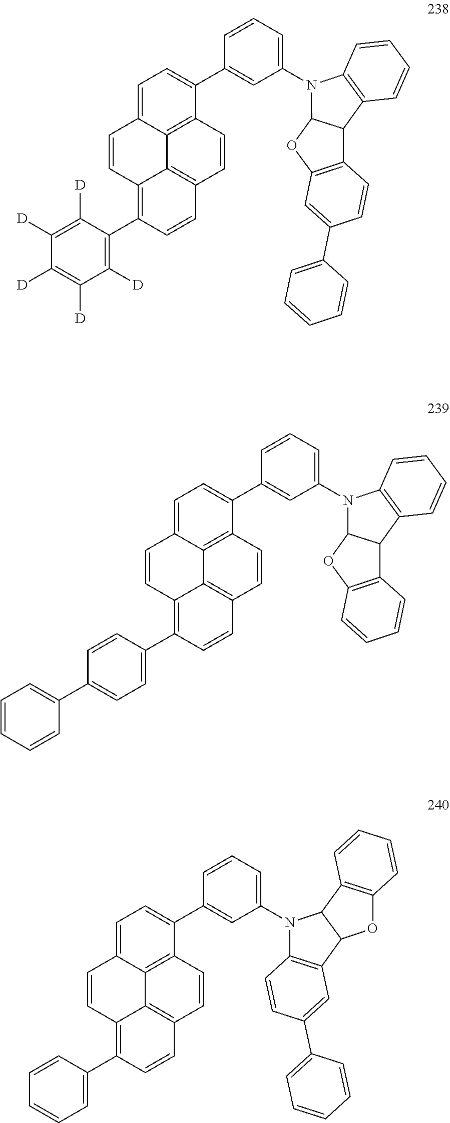 [SMILES: [2H]c1c([2H])c([2H])c(-c2ccc3ccc4c(-c5cccc(N6c7ccccc7C7c8ccc(-c9ccccc9)cc8OC76)c5)ccc5ccc2c3c54)c([2H])c1[2H] ; c1ccc(-c2ccc(-c3ccc4ccc5c(-c6cccc(N7c8ccccc8C8c9ccccc9OC87)c6)ccc6ccc3c4c65)cc2)cc1 ; c1ccc(-c2ccc3c(c2)C2Oc4ccccc4C2N3c2cccc(-c3ccc4ccc5c(-c6ccccc6)ccc6ccc3c4c65)c2)cc1]

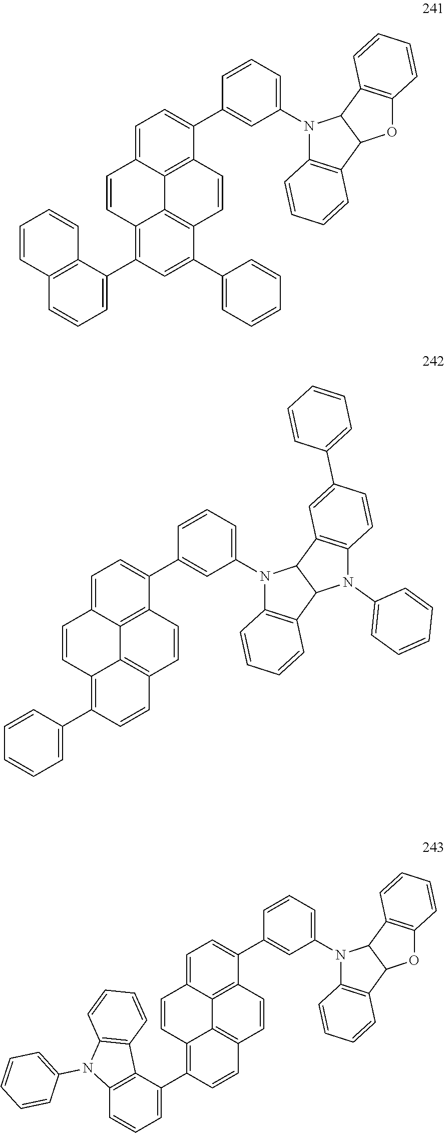 [SMILES: c1ccc(-c2cc(-c3cccc4ccccc34)c3ccc4ccc(-c5cccc(N6c7ccccc7C7Oc8ccccc8C76)c5)c5ccc2c3c45)cc1 ; c1ccc(-c2ccc3c(c2)C2C(c4ccccc4N2c2cccc(-c4ccc5ccc6c(-c7ccccc7)ccc7ccc4c5c76)c2)N3c2ccccc2)cc1 ; c1ccc(-n2c3ccccc3c3c(-c4ccc5ccc6c(-c7cccc(N8c9ccccc9C9Oc%10ccccc%10C98)c7)ccc7ccc4c5c76)cccc32)cc1]

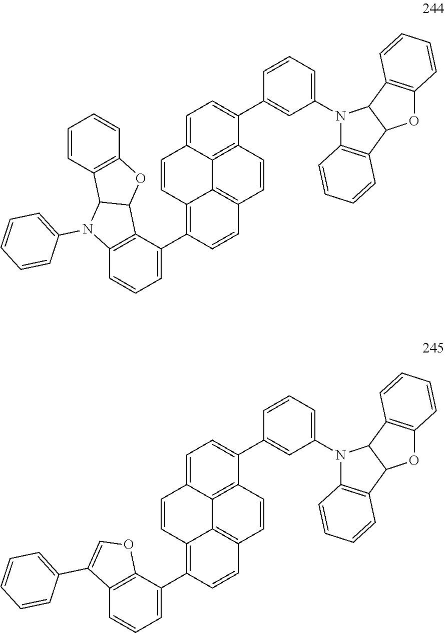 [SMILES: c1ccc(N2c3cccc(-c4ccc5ccc6c(-c7cccc(N8c9ccccc9C9Oc%10ccccc%10C98)c7)ccc7ccc4c5c76)c3C3Oc4ccccc4C32)cc1 ; c1ccc(-c2coc3c(-c4ccc5ccc6c(-c7cccc(N8c9ccccc9C9Oc%10ccccc%10C98)c7)ccc7ccc4c5c76)cccc23)cc1]

10. An organic light emitting device comprising a first electrode, a second electrode opposite to the first electrode, and one or more organic layers interposed between the first and second electrodes wherein one of the organic layers comprises the compound according to claim 1.

11. The organic light emitting device according to claim 10, wherein the organic layers comprise a hole injecting layer, a hole transport layer, a functional layer having functions of both hole injection and hole transport, a light emitting layer, an electron transport layer, an electron injecting layer, and/or a functional layer having functions of both electron injection and electron transport.

12. The organic light emitting device according to claim 10, wherein one of the organic layers interposed between the first and second electrodes is a light emitting layer composed of a host and a dopant and wherein the host is the compound represented by Formula 1 or Formula 2.

13. The organic light emitting device according to claim 12, wherein the dopant comprises a compound represented by one of Formulas D1 to D10: 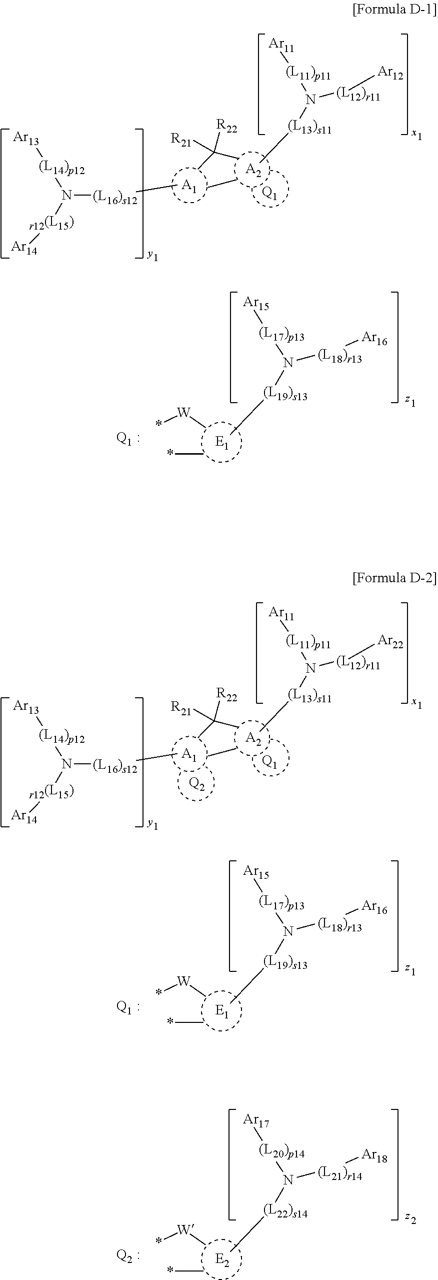
wherein A₁, A₂, E₁, and E₂ are each independently a substituted or unsubstituted C₆-C₅₀ aromatic hydrocarbon ring or a substituted or unsubstituted C₂-C₄₀ aromatic heterocyclic ring, with the proviso that two adjacent carbon atoms in the aromatic ring A₁ and two adjacent carbon atoms in the aromatic ring A₂ optionally form a 5-membered ring with the carbon atom to which R₂₁ and R₂₂ are attached, to form a fused ring, L₁₁ to L₂₂ are the same as or different from each other and are each independently selected from a single bond, substituted or unsubstituted C₁-C₆₀ alkylene, substituted or unsubstituted C₂-C₆₀ alkenylene, substituted or unsubstituted C₂-C₆₀ alkynylene, substituted or unsubstituted C₃-C₆₀ cycloalkylene, substituted or unsubstituted C₂-C₆₀ heterocycloalkylene, substituted or unsubstituted C₆-C₆₀ arylene, and substituted or unsubstituted C₂-C₆₀ heteroarylene, W, and W′ are each independently selected from N—R₂₃, CR₂₄R₂₅, SiR₂₆R₂₇, GeR₂₈R₂₉, O, S, and Se, R₂₁ to R₂₉ and Ar₁₁ to Ar₁₈ are the same as or different from each other and are each independently selected from hydrogen, deuterium, substituted or unsubstituted C₁-C₃₀ alkyl, substituted or unsubstituted C₆-C₅₀ aryl, substituted or unsubstituted C₂-C₃₀ alkenyl, substituted or unsubstituted C₂-C₂₀ alkynyl, substituted or unsubstituted C₃-C₃₀ cycloalkyl, substituted or unsubstituted C₅-C₃₀ cycloalkenyl, substituted or unsubstituted C₂-C₅₀ heteroaryl, substituted or unsubstituted C₂-C₃₀ heterocycloalkyl, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted C₁-C₃₀ alkylamine, substituted or unsubstituted C₅-C₃₀ arylamine, substituted or unsubstituted C₁-C₃₀ alkylsilyl, substituted or unsubstituted C₅-C₃₀ arylsilyl, substituted or unsubstituted C₁-C₃₀ alkylgermanium, substituted or unsubstituted C₁-C₃₀ arylgermanium, cyano, nitro, and halogen, with the proviso that R₅₁ and R₅₂ are optionally linked to each other to form an alicyclic or aromatic monocyclic or polycyclic ring in which carbon atoms are optionally substituted with one or more heteroatoms selected from the group consisting of N, O, P, Si, S, Ge, Se, and Te, p11 to p14, r11 to r14, and s11 to s14 are each independently an integer from 1 to 3, provided that when p11 is 2 or greater, the linkers L₁₁ are the same as or different from each other, when p12 is 2 or greater, the linkers L₁₄ are the same as or different from each other, when p13 is 2 or greater, the linkers L₁₇ are the same as or different from each other, p14 is 2 or greater, the linkers L₂₀ are the same as or different from each other, when r11 is 2 or greater, the linkers L₁₂ are the same as or different from each other, when r12 is 2 or greater, the linkers L₁₅ are the same as or different from each other, when r13 is 2 or greater, the linkers L₁₈ are the same as or different from each other, when r14 is 2 or greater, the linkers L₂₁ are the same as or different from each other, when s11 is 2 or greater, the linkers L₁₃ are the same as or different from each other, when s12 is 2 or greater, the linkers L₁₆ are the same as or different from each other, when s13 is 2 or greater, the linkers L₁₉ are the same as or different from each other, and when s14 is 2 or greater, the linkers L₂₂ are the same as or different from each other, x1 is 1, and y1 and z1 are each independently an integer of 0 or 1, with the proviso that Ar₁₁ and Ar₁₂ are optionally linked to each other to form a ring, Ar₁₃ and Ar₁₄ are optionally linked to each other to form a ring, Ar₁₅ and Ar₁₆ are optionally linked to each other to form a ring, and Ar₁₇ and Ar₁₈ are optionally linked to each other to form a ring, and with the proviso that two adjacent carbon atoms in the ring A₂ are optionally bonded to Q₁₁ at the asterisks (*) to form a fused ring, and with the proviso that two adjacent carbon atoms in the ring A₁ are optionally bonded to Q₁₂ at the asterisks (*) to form a fused ring and two adjacent carbon atoms in the ring A₂ are optionally bonded to Q₁₁ at the asterisks (*) to form a fused ring; 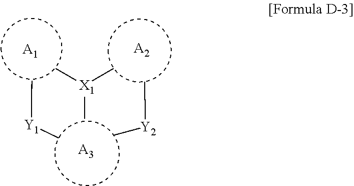
wherein X₁ is selected from B, P, and P═O, A₁ to A₃ are the same as or different from each other and are each independently a substituted or unsubstituted C₆-C₅₀ aromatic hydrocarbon ring or a substituted or unsubstituted C₂-C₄₀ aromatic heterocyclic ring, Y₁, and Y₂ are each independently selected from NR₂₁, CR₂₂R₂₃, O, S, Se, and SiR₂₄R₂₅, and R₂₁ to R₂₅ are the same as or different from each other and are each independently selected from hydrogen, deuterium, substituted or unsubstituted C₁-C₃₀ alkyl, substituted or unsubstituted C₆-C₅₀ aryl, substituted or unsubstituted C₃-C₃₀ cycloalkyl, substituted or unsubstituted C₂-C₅₀ heteroaryl, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted C₁-C₃₀ alkylamine, substituted or unsubstituted C₅-C₃₀ arylamine, substituted or unsubstituted C₁-C₃₀ alkylsilyl, substituted or unsubstituted C₅-C₃₀ arylsilyl, cyano, and halogen, with the proviso that each of R₂₁ to R₂₅ is optionally bonded to one or more rings selected from A₁ to A₃ to form an alicyclic or aromatic monocyclic or polycyclic ring; 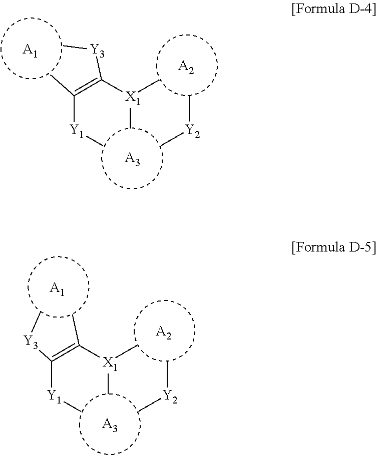
wherein Y₃ is selected from NR₂₁, CR₂₂R₂₃, O, S, Se, and SiR₂₄R₂₅, and X₁, Y₁, Y₂, A₁ to A₃, and R₂₁ to R₂₅ are as defined in Formula D3; 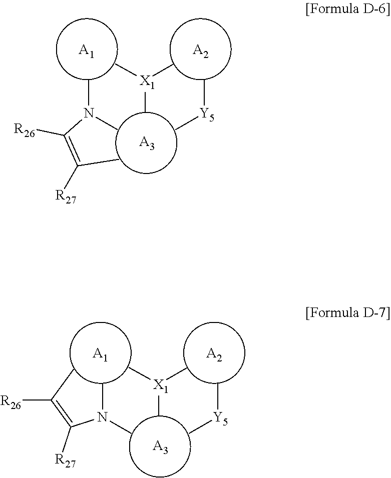
wherein Y₅ is selected from NR₂₁, CR₂₂R₂₃, O, S, Se, and SiR₂₄R₂₅, and X₁, A₁ to A₃, R₂₁ to R₂₅, R₂₆, and R₂, are as defined in Formula D3; 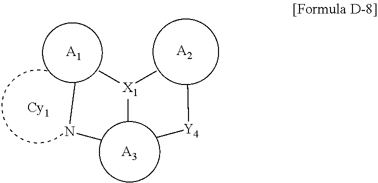
wherein Y₄ is selected from NR₂₁, CR₂₂R₂₃, O, S, Se, and SiR₂₄R₂₅, and X₁, A₁ to A₃, and R₂₁ to R₂₅ are as defined in Formula D3 and Cy₁ represents a ring formed by a substituted or unsubstituted C₂-C₅ alkylene group with the nitrogen (N) atom between the rings A₁, the aromatic carbon atom in the ring Q₁ to which the nitrogen (N) atom is bonded, and the aromatic carbon atom in the ring Q₁ to which Cy₁ is to be bonded; 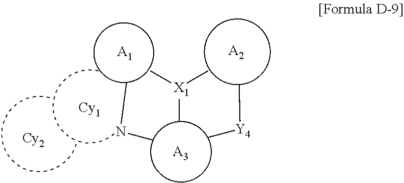
wherein Y₄ is selected from NR₂₁, CR₂₂R₂₃, O, S, Se, and SiR₂₄R₂₅, and X₁, A₁ to A₃, and R₂₁ to R₂₅ are as defined in Formula D3 and Cy₂ represents a saturated hydrocarbon ring attached to Cy₁ and formed by a substituted or unsubstituted C₂-C₅ alkylene group with carbon atoms in Cy₁; 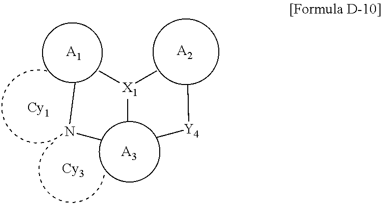
wherein Y₄ is selected from NR₂₁, CR₂₂R₂₃, O, S, Se, and SiR₂₄R₂₅, and X₁, A₁ to A₃, and R₂₁ to R₂₅ are as defined in Formula D3 and Cy₃ represents a ring formed by a substituted or unsubstituted C₁-C₄ alkylene group with the aromatic carbon atom in the ring A₃ to which Cy₃ is to be bonded, the nitrogen (N) atom between the rings A₃, the aromatic carbon atom in the ring A₃ to which the nitrogen (N) atom is bonded, and the carbon atom in Cy₁ to which the nitrogen (N) atom is bonded; and
As used herein, the term “substituted” in the definition of the substituents in Formulas D1 to D10 indicates substitution with one or more substituents selected from the group consisting of deuterium, cyano, halogen, hydroxyl, nitro, C₁-C₂₄ alkyl, C₃-C₂₄ cycloalkyl, C₁-C₂₄ haloalkyl, C₂-C₂₄ alkenyl, C₂-C₂₄ alkynyl, C₃-C₂₄ cycloalkyl, C₁-C₂₄ heteroalkyl, C₆-C₂₄ aryl, C₇-C₂₄ arylalkyl, C₇-C₂₄ alkylaryl, C₂-C₂₄ heteroaryl, C₂-C₂₄ heteroarylalkyl, C₁-C₂₄ alkoxy, C₁-C₂₄ alkylamino, C₁₂-C₂₄ diarylamino, C₂-C₂₄ diheteroarylamino, C₇-C₂₄ aryl(heteroaryl)amino, C₁-C₂₄ alkylsilyl, C₆-C₂₄ arylsilyl, C₆-C₂₄ aryloxy, and C₆-C₂₄ arylthionyl, more preferably deuterium, cyano, halogen, hydroxyl, nitro, C₁-C₁₂ alkyl, C₁-C₁₂ haloalkyl, C₂-C₁₂ alkenyl, C₂-C₁₂ alkynyl, C₃-C₁₂ cycloalkyl, C₁-C₁₂ heteroalkyl, C₆-C₁₈ aryl, C₇-C₂₀ arylalkyl, C₇-C₂₀ alkylaryl, C₂-C₁₈ heteroaryl, C₂-C₁₈ heteroarylalkyl, C₁-C₁₂ alkoxy, C₁-C₁₂ alkylamino, C₁₂-C₁₈ diarylamino, C₂-C₁₈ diheteroarylamino, C₇-C₁₈ aryl(heteroaryl)amino, C₁-C₁₂ alkylsilyl, C₆-C₁₈ arylsilyl, C₆-C₁₈ aryloxy, and C₆-C₁₈ arylthionyl, or a combination thereof. The term “unsubstituted” in the same definition indicates having no substituent. One or more hydrogen atoms in each of the substituents are optionally replaced by deuterium atoms.

14. The organic light emitting device according to claim 11, wherein each of the organic layers is formed by a deposition or solution process.

15. The organic light emitting device according to claim 10, wherein the organic light emitting device is used in a display or lighting system selected from flat panel displays, flexible displays, monochromatic flat panel lighting systems, white flat panel lighting systems, flexible monochromatic lighting systems, flexible white lighting systems, displays for automotive applications, displays for virtual reality, and displays for augmented reality.
